(19) United States
(12) Patent Application Publication
(10) Pub. No.: US 2025/0051813 A1
(43) Pub. Date: Feb. 13, 2025

(54) METABOLICALLY ENGINEERED ORGANISMS FOR THE PRODUCTION OF ADDED VALUE BIO-PRODUCTS

(71) Applicant: Inbiose N.V., Zwijnaarde (BE)

(72) Inventors: Jo Maertens, Gent (BE); Joeri Beauprez, Bredene (BE); Marjan De Mey, Gent (BE)

(21) Appl. No.: 18/814,011

(22) Filed: Aug. 23, 2024

Related U.S. Application Data
(62) Division of application No. 17/061,741, filed on Oct. 2, 2020, now Pat. No. 12,098,403.
Application No. 17/061,741 is a division of application No. 16/599,921, filed on Oct. 11, 2019, now Pat. No. 11,384,374.
Application No. 16/599,921 is a division of application No. 15/224,047, filed on Jul. 29, 2016, now Pat. No. 10,570,430.
Application No. 15/224,047 is a division of application No. 13/809,340, filed on Jan. 9, 2013, now Pat. No. 9,701,992.

(30) Foreign Application Priority Data
Jul. 12, 2010 (EP) 10169304.2

(51) Int. Cl. C12P 19/14; C12N 9/10; C12N 9/26; C12N 9/90; C12N 15/52; C12N 15/70; C12N 15/81; C12P 19/18; C12P 19/30

(52) U.S. Cl. CPC C12P 19/14; C12N 9/1051; C12N 9/2431; C12N 9/90; C12N 15/52; C12N 15/70; C12N 15/81; C12P 19/18; C12P 19/305; C12Y 204/01007; C12Y 204/01008; C12Y 204/01022; C12Y 204/01069; C12Y 302/01026; C12Y 504/02002

(57) ABSTRACT

The present invention relates to genetically engineered organisms, especially microorganisms such as bacteria and yeasts, for the production of added value bio-products such as specialty saccharide, activated saccharide, nucleoside, glycoside, glycolipid or glycoprotein. More specifically, the present invention relates to host cells that are metabolically engineered so that they can produce said valuable specialty products in large quantities and at a high rate by bypassing classical technical problems that occur in biocatalytical or fermentative production processes.

### RELATED APPLICATIONS

This application is a divisional of U.S. patent application Ser. No. 17/061,741, filed Oct. 2, 2020, which is a divisional of U.S. patent application Ser. No. 16/599,921, filed Oct. 11, 2019, now U.S. Pat. No. 11,384,374, which is a divisional of U.S. patent application Ser. No. 15/224,047, filed Jul. 29, 2016, now U.S. Pat. No. 10,570,430, which is a divisional of U.S. patent application Ser. No. 13/809,340, filed Jan. 9, 2013, now U.S. Pat. No. 9,701,992, which is a 35 U.S.C. § 371 filing of International Patent Application No. PCT/EP2011/061891, filed Jul. 12, 2011, which claims priority to European Patent Application No. 10169304.2, filed Jul. 12, 2010, the entire disclosures of which are hereby incoporated herein by reference.

### SEQUENCE LISTING

The instant application contains a Sequence Listing which has been submitted electronically in XML format and is hereby incorporated by reference in its entirety. Said XML file, created on Aug. 20, 2024, is named 756525_DCV-001USDIV4_ST26.xml and is 84,286 bytes in size.

### TECHNICAL FIELD OF INVENTION

The present invention relates to genetically engineered organisms, especially microorganisms such as bacteria and yeasts, for the production of added value bio-products such as specialty saccharides, glycolipids and glycoproteins. More specifically, the present invention relates to host cells that are metabolically engineered so that they can produce said valuable specialty products in large quantities and at a high rate by bypassing classical technical problems that occur in biocatalytical or fermentative production processes.

### BACKGROUND ART

The increasing cost of petroleum resources contributes to a growing awareness of the potential of biological production processes. This has intensified the research efforts of companies and research centres towards the development of economically viable and environmentally benign technologies for the production of an increasing number of bio-products, e.g., bio-fuels, bio-chemicals and bio-polymers. These are easily degradable and produced with minimal energy requirements and waste streams. In spite of the favourable context for production processes based on industrial biotechnology, the development of alternatives for well-established chemical synthesis routes often is too time intensive and too expensive to be economically viable.

Consequently, there is a clear demand for a faster and cheaper development of new production strains.

Nowadays oligosaccharides are typically synthesized via bioconversion processes. Isolated and purified enzymes (so called in vitro bioconversions) and whole cell biocatalysts are commonly used. In essence, they convert one or more precursors into a desired bio-product.

However, the application of the in vitro bioconversions is often hampered because the synthesis of the product may require multiple enzymatic steps or because additional cofactors are required (NADH, NADPH, UTP, . . . ), which are expensive.

Another drawback of in vitro synthesis is the fact that the expression and purification of many enzymes is laborious and their purification process may result in a decreased enzymatic activity. Furthermore, each enzyme in such a multi-enzyme bioconversion process has its own optimal process parameters, resulting in very complicated optimization schemes. In such a process, the reaction equilibria also play an important role. For instance, when using a phosphorylase, a set substrate/product ratio that limits product yield will be at hand. This leads to complicated downstream processing schemes to separate the product from the substrate (33, 35).

Metabolic engineering is another approach to optimize the production of value added bio-products such as specialty carbohydrates. Commonly, whole cells have been metabolically engineered to produce added value bio-products starting from a supplied precursor. In this context, the cells are engineered as such that all the metabolic pathways involved in the degradation of the precursor(s) are eliminated (3, 45, 70, 77, 100). By doing so, the precursor(s) is (are) efficiently and directly converted into the desired product.

A major drawback of the latter approach is the fact that the biomass synthesis and the envisaged bio-product biosynthesis require different starting metabolites. For example, E. coli was metabolically engineered for the efficient production of 2-deoxy-scyllo-inosose starting from glucose. This strategy renders the metabolically engineered E. coli unfit to grow on glucose, requiring the addition of other substrates, e.g., glycerol to allow for biomass synthesis (45).

A second drawback of whole cell production systems is that there is a need for two phases, a growth phase, in which biomass is formed (or biomass synthesis), followed by a production phase of the envisaged product. This means that the growth phase and the production phase are separated in the time (consecutive phases). This results in very low overall production rates of the desired product(s). In addition, this type of process is hard to optimize. Indeed, fermentation processes have been developed making use of metabolically engineered cells which over-express production pathway genes. A large amount of the substrate is converted into biomass, resulting in only a minor flux of the substrate towards the product (13).

The present invention overcomes the above-described disadvantages as it provides metabolically engineered organisms which are capable to produce desired products with a high productivity and a guaranteed high yield (FIG. 1). This is accomplished by clearly splitting the metabolism of the organism in two parts: 1) a so-called ‘production part’ or ‘production pathway’, and 2) a ‘biomass and cofactor supplementation’ part or ‘biomass and/or bio-catalytic enzyme formation pathway’. Said two parts are created by splitting a sugar into: a) an activated saccharide, and b) a (non-activated) saccharide. Each of said saccharides a) or b) are—or can be—the first precursors of either the production pathway a) or biomass and/or bio-catalytic enzyme formation pathways b), allowing a pull/push mechanism in the cell.

Indeed, biomass synthesis, which is the main goal of the cell, converts the activated saccharide or the saccharide into biomass and shifts the equilibrium of the reaction that splits the sugar towards the activated saccharide and saccharide. In this way, the life maintaining drive of the cell acts as a pulling mechanism for the product pathway. This pulling effect is created by biomass synthesis as it ensures the accumulation of the first substrate molecule of the production pathway, which, as such and in turn, also pushes the production pathway. This strategy solves the production rate problem which occurs in the two phase production strategies as described in the prior art. Moreover, by catabolising one part of the sugar moiety, the cell is always supplied with the necessary cofactors and the needed energy requirements for production of the specialty bio-product. The current strategy thus solves also the problem of co-factor supplementation that is needed in biocatalytic production as described in the prior art. In addition, the necessary enzymes in the production pathway are always synthesized efficiently and easily maintained via the engineering strategy of the current invention.

In addition, the present invention discloses the usage of a 2-fucosyltransferase originating from Dictyostellium discoideum to produce 2-fucosyllactose by the metabolically engineered organisms of the present invention.

### BRIEF DESCRIPTION OF FIGURES

FIG. 1: (a) A normal equilibrium reaction, which occurs in the current production technologies (b) pull/push principle: the equilibrium is shifted towards the saccharide and activated saccharide. The main goal of a cell is to grow, hence it pulls at the saccharide (by means of example), to form biomass (“biomass and/or bio-catalytic enzyme formation pathway”) and supplements the necessary co-factors for the production pathway next to life maintenance. Due to this pulling effect, the activated saccharide will accumulate in the cell and pushes the production pathway.

FIG. 2: Growth rate of E. coli transformants on minimal medium carrying a plasmid encoding for a sucrose phosphorylase. (Abbreviations are given in Table 2).

FIG. 3: Projected carbon flow in the wild type strain. Small arrow: Indicating reactions in the metabolism. Bold arrow: Indicating enhanced or novel introduced reactions. Cross: indicates the knocking-out of a gene or rendering it less functional.

FIG. 4: Projected carbon flow in Base strain 1. The αglucose-1-phosphate (αG1P) pool in Base strain 1 increases because the main reactions that convert αG1P into cellular components are eliminated. Small arrow: Indicating reactions in the metabolism. Bold arrow: Indicating enhanced or novel introduced reactions. Cross: indicates the knocking-out of a gene or rendering it less functional.

FIG. 5: The αglucose-1-phosphate pool in the wild type E. coli MG1655 (WT) grown on glucose, E. coli MG1655 P22-BaSP grown on sucrose, and in the plug strain MG1655 ΔglgC Δpgm ΔlacZ P22-BaSP grown on sucrose: shake flask experiments and 1.5 L batch experiments.

FIG. 6: Evolution of sucrose, acetate and the biomass concentration during a culture of ΔpgmΔlacZΔglgC (3KO) P22-BaSP on buffered LB medium.

FIG. 7: Evolution of αGlucose-1-phosphate, fructose, glucose, and pyruvate concentration during a culture of ΔpgmΔlacZΔglgC (3KO) P22-BaSP on buffered LB medium.

FIG. 8: Schematic view of the cellobiose producer ΔpgmΔlacZΔglgC (3KO) P22-BaSP-CuCP.

FIG. 9: Cellobiose production and yield of ApgmAlacZAglgCΔagp (4KO) P22-BaSP-CuCP. High phosphate indicates a phosphate concentration of 0.2 M phosphate, low phosphate indicates a concentration of 13 mM phosphate.

FIGS. 10A and 10B: Starting from base strain 5, fucosylated sugar derivates such as fucosyllactose and more specifically 1,2-fucosyllactose can be produced. The strain is modified to force the cell to produce frucose-6-phosphate which is an intermediate in the synthesis of GDP-fucose. Glucose or glucose-1-phosphate (if the starting enzyme is either a sucrase or a sucrose phosphorylase) is then fed to the central carbon metabolism via the pentose phosphate pathway. FIG. 10A shows the optimal route towards product and biomass and the needed knock outs to achieve this with a sucrose phosphorylase. FIG. 10B shows the optimal route with an invertase combined with glucokinase.

FIG. 11: The sequence shown (SEQ ID NO: 51) is the partial genome sequence of wild type chromosome at the location of the pgm gene. The pgm gene sequence is marked in bold/italic.

FIG. 12: The sequence shown (SEQ ID NO: 52) is the partial genome sequence of a mutant strain in which only a scar remains at the location of the pgm gene. The scar sequence is marked in bold/italic.

FIG. 13: The sequence shown (SEQ ID NO: 53) is the partial genome sequence of a pgm mutant strain in which the pgm gene is replaced with a part of a GFP protein sequence. The newly introduced sequence is marked in bold/italic.

FIG. 14: The sequence shown (SEQ ID NO: 54) is the partial genome sequence of a pgm mutant strain in which the pgm gene is replaced with kanamycine cassette. The newly introduced sequence is marked in bold/italic.

FIG. 15: The amount of glucose that was released after polysaccharide hydrolysis for the wild type strains transformed with a heterologous sucrose phosphorylase originating from Bifidobacterium adolescentis and a mutant strain ΔpgmΔagpΔglgCΔlacZΔglkΔptsG with a heterologous sucrose phosphorylase originating from Bifidobacterium adolescentis and a heterologous gene tts originating from Streptococcus pneumoniae.

FIG. 16: Kojibiose, maltose and optical density evolution in time of a kojibiose producing strain with the genotype ΔlacZΔglgCΔagpΔptsGΔmalPQΔycjU pCXp22MPp22KP.

FIG. 17: Growth profile and F6P accumulation of a ΔpgiΔpfkAΔpfkB mutant strain in a sucrose based medium.

FIG. 18: Phylogenetic tree of the fucosyltransferases from the different glycosyltransferase families, the tree was constructed with MCoffee (58).

FIG. 19: Alignment of Dictyostelium discoideum and Helicobacter pylori fucosyltransferase, the amino acids marked in colour are conserved motives found in the 2-fucosyltransferases of GT family 11. Bold indicates motif 1, underlined, motif 2, and italic, motif 3 (67). The depicted Dictyostelium discoideum sequence shown corresponds with SEQ ID NO: 55. The depicted Helicobacter pylori sequence shown corresponds with SEQ ID NO: 56.

FIGS. 20A and 20B: LC-MS results of the fucosyltransferase assay with phenol red. The chromatogram in FIG. 20A is the blanc without GDP-fucose. The chromatogram in FIG. 20B is a sample of the D. discoideum fucosyltransferase assay; a 13.5 min a peak of 2FL appeared in the chromatogram.

FIG. 21: 2-fucosyllactose LC MSMS analysis of the partial fucosyltransferase enzyme. The upper chromatogram shows a 100 mg/l standard of 2-fucosyllactose, the lower chromatogram shows the 2-fucosyllactose peak of the enzyme assay. In this analysis only the mass of 2-fucosyllactose was scanned with the mass spectrometer.

### DESCRIPTION OF THE INVENTION

The present invention discloses metabolically engineered organisms, especially microorganisms, which are capable to produce added value bio-products with a high productivity and a guaranteed high yield. The organisms of the present invention are metabolically engineered so that two distinct routes are constructed leading to product and to growth/biomass. This is achieved by reducing or eliminating the activity of enzymes catalyzing reactions converting metabolites from the ‘biomass and/or bio-catalytic enzyme and/or cofactor supplementation part’ into metabolites from the ‘production pathway’ part and vice versa, e.g. by reducing or eliminating/knocking-out at least one, some or all the genes encoding for enzymes performing reactions which convert the production pathway intermediates into biomass precursors, and/or reducing or eliminating/knocking out at least one, some or all the genes coding for enzymes performing the reactions which degrade the production pathway intermediates. Moreover, these metabolic/genetic changes do not impair the growth of the engineered cells. For example: carbohydrate hydrolases in combination with carbohydrate kinases, carbohydrate synthases, and, carbohydrate phosphorylases can be introduced into the cell. The latter enzymes convert the substrates comprising a saccharide, an oligosaccharide, a polysaccharide or a mixture thereof in a sugar moiety and an activated sugar moiety (e.g. a phosphorylated saccharide moiety, UDP, CMP, GDP, ADP, TDP or dTDP . . . activated sugar moiety). Additional metabolic engineering of the cell involves blocking of the pathway starting from the activated sugar moiety towards the biomass constituents. In this way, the (non-activated) sugar moiety is used as ‘fuel’ (or energy-source) and building block for the synthesis of biomass, of the numerous bio-catalytic enzymes that will perform the conversion of the activated sugar moiety to the desired product (=e.g. a specialty carbohydrate) and of the necessary cofactors (NADH, ATP, UTP . . . ). Conversely, the activated sugar moiety can also be used as ‘fuel’, while the sugar moiety is activated by a carbohydrate kinase and ‘pushed’ into the production route of the desired specialty product.

Using the engineered organisms of the present invention, product formation through the conversion of the activated sugar can be linked to growth which is fuelled by the other sugar moiety (or vice versa). In this way, the cell's natural drive for multiplication is used as an asset to push the production of the desired bio-product. This means that the former drawback of having to produce biomass before the actual production of the bio-product can start, is now turned into a benefit. This methodology results in high production rates, without the inherent problems that come with multi-enzymes systems and two phase fermentation systems. In addition, the organisms of the present invention may use the same substrate(s) as indicated above for both growth or biomass production and production of the desired product at a high rate, the overall principle behind this metabolic engineering strategy is thus a pull/push principle as is also explained above. The central carbon metabolism that leads to biomass and cofactors pulls at one part of the sugar moiety for growth while the other part accumulates in the cell, pushing the production pathway.

The latter approach cannot only be used to produce desired specialty carbohydrates or activated carbohydrates but can also be applied for the synthesis of a wide variety of glycosylated compounds, e.g., saccharides, nucleosides, glycosylphosphates, glycoproteins and glycolipids. Multiple starting enzymes can be introduced into a cell to split the metabolism into two parts, in combination with gene knock outs. Non-limiting examples of enzymes that can be used to split sugars into an activated saccharide and a saccharide are sucrose phosphorylases, sucrose synthases, sucrases (invertases) combined with a glucokinase and/or fructokinase, a trehalase combined with a glucokinase, a maltase combined with a glucokinase, a sucrose-6-phosphate hydrolase combined with a fructokinase, a maltose phosphorylase, a maltose synthase, a amylase combined with a phosphorylase or synthase or hydrolase, a lactose synthase, a lactose phosphorylase, a lactase (or beta-galactosidase) combined with a galactokinase and/or a glucokinase.

The present invention relates to a metabolically engineered organism for the production of at least one specialty product chosen from the group consisting of a saccharide, an activated saccharide, a nucleoside, a glycoside, a glycolipid and a glycoprotein, characterized in that:
a) said organism is genetically modified with the introduction of at least: i) a gene encoding for a carbohydrate hydrolase in combination with a gene encoding for a carbohydrate kinase, ii) a gene encoding for a carbohydrate synthase, or, iii) a gene encoding for a carbohydrate phosphorylase, so that said organism is capable to split a disaccharide, oligosaccharide, polysaccharide or a mixture thereof into an activated saccharide and a saccharide, and
b) said organism is further genetically modified so that at least one other gene than any of the introduced genes of step a) of said organism is rendered less-functional or non-functional and wherein said other gene encodes for an enzyme which converts said activated saccharide into biomass and/or bio-catalytic enzymes.

The term ‘saccharide’ relates to monosaccharides such as, but not limited to, aldoses, ketoses, pentoses, methylpentoses, hexoses, polyols with or without either carbonyl, carboxyl, amino groups or in which a hydroxylgroup is replaced by, but not limited to a hydrogen, amino, thiol, phosphate and/or similar group or a derivative of these groups. The term ‘saccharide’ also relates to di-, oligo-, and polysaccharide which are made up of one or more monosaccharides as described above, linked to each other by a glycosidic bond.

The term ‘nucleoside’ relates to each monosaccharide that is substituted with a nucleotide which is for instance, but not limited to, UDP, GDP, ADP, TDP, CMP, or dTDP.

The term ‘glycoside’ relates to a saccharide which forms a glycosidic bond with other chemical compounds, such as, but not limited to sterols, phenols, fatty acids, phosphatidylinositols, vitamine C, cartenoides and artimisinine.

The term ‘glycolipid’ relates to a saccharide which forms a glycosidic bond with a fatty acid or lipid.

The term ‘glycoprotein’ relates to a saccharide which forms a glycosidic bond with a protein.

The term ‘glycosylphosphate’ relates to a phosphorylated saccharide.

The present invention further relates to an organism as indicated above wherein said organism is further genetically modified with the introduction of at least one other gene which converts said activated saccharide into a specialty product, or, wherein at least one other endogenous gene of said organism which converts said activated saccharide into a specialty product is over-expressed.

In addition, the present invention relates to an organism as indicated above wherein said organism is capable to grow on a disaccharide, oligosaccharide, polysaccharide or a mixture thereof as the main carbon source. With the term ‘main’ is meant the most important carbon source for biomass formation, i.e. 75, 80, 85, 90, 95, 98, 99% of all the required carbon is derived from the above-indicated carbon source. In one embodiment of the invention, said carbon source is the sole carbon source for said organism, i.e. 100% of all the required carbon is derived from the above-indicated carbon source.

The term ‘metabolic engineering’ refers to the practice of optimizing genetic and regulatory processes within said organism to increase the organism's production of a certain desired substance or product. The latter product is hereby denominated as a ‘specialty product’ and specifically relates to a desired saccharide (activated or non-activated), a nucleoside, a glycoside, a glycolipid or a glycoprotein. Some non-limiting examples of such products are sugar derivates such as 1,2-fucosyllactose, 1,3-fucosyllactose, 1,4-fucosyllactose, 1,6-fucosyllactose, galactinol, stachyose, globotriose, galactose (beta1-4) rhamnose, sophorose, cellobiose, UDP-glucose, sophorolipids, myo-inositol, L-arabinose, scyllo-inosose, glycosylphosphatidylinositol, lacto-N-biose, lacto-N-tetraose, lactosamine, fucosylated galactosyloligosaccharides, L-fucose. N-Ac glucosamine, sialic acid, sialyllactose, chitosan and chitin.

The term ‘engineering’ relates to any well-known technique which can be used to genetically modify an organism as is for example described in (9, 17, 19, 21, 22, 42).

The terms ‘an organism being capable to grow on a disaccharide, oligosaccharide, polysaccharide or a mixture thereof as the main carbon source’ means that organisms of the present invention may use the same disaccharide, oligosaccharide, polysaccharide or a mixture thereof for both growth (or biomass production) and production of the desired product, and, that they can use the latter saccharides as the only carbon source in order to multiply and/or to be metabolically active. In short, the organisms of the present invention are capable to multiply and metabolize in or on a medium comprising said saccharides as the only carbon source.

With the terms ‘splitting (or conversion) into an activated saccharide and a saccharide’ is meant that the latter saccharides which are used as carbon source will be split (or converted) by the organism of the present invention into an activated sugar moiety -some non-limiting examples of activated sugar moieties are sugars moieties bearing a phosphate, UDP, GDP, ADP, TDP or dTDP group- and a non-activated sugar moiety which does not bear or is not bound to the latter groups.

The terms ‘biocatalytic enzymes’ refers to all enzymes needed for the production of the specialty carbohydrate.

The term ‘biomass’ refers to all cellular components (i.e. proteins, DNA, RNA, phosphatidylserine, phosphatidylethanolamine, cardiolipin, phosphatidylglycerol, putrescine, spermidine, peptidoglycan, glycogen and/or lipopolysacharide (63) that can be synthesized in the modified specialty carbohydrate production strain from the sugar moiety that is not used in the specialty carbohydrate (and other added value products) production route.

The terms ‘genes which are rendered less-functional or non-functional’ refer to the well-known technologies for a skilled person (such as the usage of siRNA, RNAi, miRNA, asRNA, mutating genes, knocking-out genes, transposon mutagenesis, . . . ) which are used to change the genes in such a way that they are less-able (i.e. statistically significantly ‘less-able’ compared to a functional wild-type gene) or completely unable (such as knocked-out genes) to produce functional final products (2, 4, 5, 7, 8, 14, 19, 37, 40, 47, 73, 79, 80, 85, 93, 98).

The term ‘(gene) knockout’ thus refers to a gene which is rendered non-functional.

The term ‘polysaccharide’ refers to a saccharide which contains 6 or more monosaccharide subunits.

The present invention further relates to a metabolically engineered organism as indicated above wherein the genetic modification of step a) is optional, or, is replaced by over-expressing at least: i) an endogenous gene encoding for a carbohydrate hydrolase in combination with an endogenous or heterologous gene encoding for carbohydrate kinase, ii) an endogenous gene encoding for a carbohydrate synthase, or, iii) an endogenous gene encoding for a carbohydrate phosphorylase, and wherein said organism is capable to split a disaccharide, oligosaccharide, polysaccharide or a mixture thereof into an activated saccharide and a saccharide.

A preferred carbohydrate hydrolase of the present invention is a lactase, invertase, sucrose, trehalase, sucrose-6-phosphate hydrolase, maltase or amylase. A preferred carbohydrate kinase of the present invention is galactokinase, a fructokinase, a glucokinase or a mannokinase.

The present invention further relates to a metabolically engineered organism as indicated above wherein said activated saccharide in step b) is replaced by said saccharide. Hence, the ‘activated sugar moiety’ in this embodiment is used as ‘fuel’, whereas the ‘sugar moiety’ is activated by a kinase and is pushed into the production route of the desired specialty product.

The present invention also relates to a metabolically engineered organism as indicated above wherein said gene in step a) splits a disaccharide, oligosaccharide, or polysaccharide in two similar or different activated saccharides or in two similar or different saccharides.

The term ‘organism’ as indicated above refers to a microorganism chosen from the list consisting of a bacterium, a yeast or a fungus cell, or, refers to a plant or animal cell. The latter bacterium preferably belongs to the species Escherichia coli. The latter yeast preferably belongs to the species Saccharomyces cereviseae.

More specifically, the present invention relates to a metabolically engineered organism as indicated above, wherein said activated saccharide is selected from the group consisting of alpha glucose-1-phosphate, alpha galactose-1-phosphate, beta glucose-1-phospate, beta galactose-1-phosphate, fructose-6-phosphate, glucose-6-phosphate, UDP-glucose and UDP-galactose and wherein said saccharide is selected from the group consisting of fructose, glucose and/or galactose.

The present invention further relates, as indicated above, to a metabolically engineered organism as indicated above, wherein said carbohydrate hydrolase is a lactase, invertase, sucrase, maltase, trehalase, sucrose-6-phosphate hydrolase and/or amylase, and, wherein said carbohydrate kinase is a galactokinase, a fructokinase, a glucokinase and/or mannokinase.

Even more specifically, the present invention relates to a metabolically engineered organism as indicated above wherein:
said gene in step a) is encoding for a sucrose phosphorylase, and/or
the following genes in step b) are rendered non-functional: a gene encoding for a beta-galactosidase, and/or, a gene encoding for a phosphoglucomutase, and/or, a gene encoding for a glucose-1-phosphate adenylyltransferase, and/or, a gene encoding for a phosphatase (such as but not limited to a glucose-1-phosphatase) and/or, a gene encoding for a glucose-1-phosphate uridyltransferase, and/or, a gene encoding for a UDP-glucose-4-epimerase, and/or, a gene encoding for UDP-glucose: galactose-1-phosphate uridyltransferase, and/or, a gene encoding for UDP-galactopyranose mutase, and/or, a gene encoding for UDP-galactose: (glucosyl) lipopolysaccharide-1,6-galactosyltransferase, and/or, a gene encoding for a UDP-galactosyltransferase, and/or, a gene encoding for a UDP-glucosyltransferase, and/or, a gene encoding for an UDP-glucuronate transferase, and/or, a gene encoding for an UDP-glucose lipid carrier transferase, and/or, a gene encoding for an UDP-sugar hydrolase, and/or, a gene encoding for an invertase, and/or, a gene encoding for a maltase, and/or, and/or a gene encoding for a trehalase, and/or, a gene encoding for a sugar transporting phosphotransferase, and/or, a gene encoding for a hexokinase.

An example of the latter metabolically engineered organism is an organism wherein:
said gene in step a) is the gene encoding for a sucrose phosphorylase possibly (but not solely) originating from a Lactic acid bacterium such as Bifidobacterium adolescentis, Leuconostoc mesenteroides, Lactobacillus acidophilus, and/or Streptococcus mutans, and/or
said genes in step b) are: gene lacZ, the gene pgm, the gene ycjU, the gene glgC, the gene agp, the gene ptsG, the gene glk, the gene glf, the gene waaB, the gene ushA, the gene wcaA, the gene wcaC, the gene wcaE, the gene wcal, the gene wcaL, the gene wcaJ, the gene galU, the gene galF, the gene galE, the gene malP, the gene malQ, and/or, the gene galT (20,25, 27, 28, 32, 46, 48, 49, 52, 54, 62, 82, 86, 87, 96, 97).

Another example of a metabolically engineered organism as indicated above is an organism wherein:
said gene in step a) is encoding for a sucrose phosphorylase possibly (but not solely) originating from a Lactic acid bacterium such as Bifidobacterium adolescentis, Leuconostoc mesenteroides, Lactobacillus acidophilus, and/or Streptococcus mutans, and/or
said genes in step b) are: the gene PGM1, the gene PGM2, the gene GLG1, the gene GLG2, the gene INM1, the gene INM2, the gene GLK1, the gene HXK1, the gene HXK2, the gene GAL10, the gene GAL7, the gene YHL012W, the gene UGP1, the gene GSY1, the gene GSY2, the gene DIE2, the gene ALG8, the gene ATG26, the gene SUC2, the gene MAL32, the gene MAL12, the gene YJL216C, and/or, the gene YGR287C, and/or, FEN1, and/or, FKS1, and/or, GSC2, and/or, TPS1 (10, 12, 15, 16, 18, 24, 30, 31, 36, 41, 51, 56, 57, 59, 61, 66, 76, 81, 84, 89-91, 99).

The latter engineered organisms can, for example but not limited to, be used to produce cellobiose, kojibiose, threhalose, L-arabinose, myo-inositol, raffinose, stachyose, L-rhamnose or L-ribose as a specialty product.

A further aspect of the present invention relates to a metabolically engineered organism as indicated above wherein said sucrose phosphorylase of step a) is replaced by a sucrose synthase, a maltose phosphorylase or a maltose synthase, or, wherein said sucrose phosphorylase of step a) is replaced by a lactose phosphorylase or a lactose synthase. The latter organisms can, for example but limited to, be used to produce sophorose, UPD-glucose, glycolipids, flavone 3-O-β-D-glucoside (sucrose synthase in step a), galactose (beta1-4)rhamnose (lactose phosphorylase in step a), or, UDP-galactose, galactinol, stachyose or globotriose, psychosine (lactose synthase in step a) as specialty products.

The present invention further relates to a metabolically engineered organism as indicated above wherein said activated saccharide in step b) is replaced by said saccharide and wherein:
said gene is step a) is encoding for a sucrose phosphorylase, and/or
the following genes in step b) are rendered non-functional: a gene encoding for a beta-galactosidase, and/or, a gene encoding for a glucose-6-phosphate isomerase, and/or, a gene encoding for a glucose-1-phosphate adenylyltransferase, and/or, a gene encoding for a phosphatase (for example, glucose-1-phosphatase), and/or, a gene encoding for a phosphofructokinase A, and/or, a gene encoding for a phosphofructokinase B, and/or, a gene encoding for phosphogluconate dehydratase, and/or, a gene encoding for 2-keto-3-deoxygluconate-6-phosphate aldolase, and/or, a gene encoding for a glucose-1-phosphate uridyltransferase, and/or, a gene encoding for an UDP-glucose-4-epimerase, and/or, a gene encoding for an UDP-glucose:galactose-1-phosphate uridyltransferase, and/or, a gene encoding for an UDP-galactopyranose mutase, and/or, a gene encoding for an UDP-galactose: (glucosyl)lipopolysaccharide-1,6-galactosyltransferase, and/or, a gene encoding for an UDP-galactosyltransferase, and/or, a gene encoding for an UDP-glycosyltransferase, and/or, a gene encoding for an UDP-glucuronate transferase, and/or, a gene encoding for an UDP-glucose lipid carrier transferase, and/or, a gene encoding for GDP-mannose hydrolase, and/or, a gene encoding for an UDP-sugar hydrolase, and/or, a gene encoding for a mannose-6-phosphate isomerase, and/or, a gene encoding for an UDP-N-acetylglucosamine enoylpyruvoyl transferase, and/or, a gene encoding for an UDP-N acetylglucosamine acetyltransferase, and/or, a gene encoding for an UDP-N-acetylglucosamine-2-epimerase, and/or, a gene encoding for an undecaprenyl-phosphate-alfa-N-acetylglucosaminyl transferase, and/or, glucose-6-phosphate-1-dehydrogenase, and/or, a gene encoding for a L-glutamine:D-fructose-6-phosphate aminotransferase, and/or, a gene encoding for a mannose-6-phosphate isomerase, and/or a gene encoding for a sorbitol-6-phosphate dehydrogenase, and/or, a gene encoding for a mannitol-1-phosphate 5-dehydrogenase, and/or a gene encoding for a allulose-6-phosphate 3-epimerase, and/or, a gene encoding for an invertase, and/or, a gene incoding for a maltase, and/or, and/or a gene encoding for a trehalase, and/or, a gene encoding for a sugar transporting phosphotransferase, and/or, a gene encoding for a hexokinase.

More specifically, the present invention relates to a metabolically engineered organism as indicated above wherein:
said gene in step a) is the sucrose phosphorylase originating from a Lactic acid bacterium such as Bifidobacterium adolescentis, Leuconostoc mesenteroides, Lactobacillus acidophilus, or Streptococcus mutans, and/or
said genes in step b) are: the gene lacZ, the gene pgi, the gene glgC, the gene agp, the gene pfkA, the gene pfkB, the gene waaB, the gene ushA, the gene eda, the gene edd, the gene wcaA, the gene wcaC, the gene wcaE, the gene wcal, the gene wcaL, the gene wcaJ, the gene wcaB, the gene wcaF, the gene wcak, the gene wcaD, the gene galU, the gene galF, the gene galE, the gene gmm, the gene galT, the gene manA, the gene murA, the gene IpxA, the gene rffE, and/or, the gene rfe, the gene glmS, the gene srlD, the gene mtID, the gene alsE and/or, zwf (6, 20, 25, 28, 29, 32, 43, 44, 46, 49, 52-55, 62, 65, 75, 78, 82, 86, 87, 96, 97, 101).

Another metabolically engineered organism according to the present invention is an organism wherein:
said gene in step a) is a gene encoding for a sucrose phosphorylase originating from a Lactic acid bacterium such as Bifidobacterium adolescentis, Leuconostoc mesenteroides, Lactobacillus acidophilus, or Streptococcus mutans, and/or
said genes in step b) are: the gene PGI1, the gene PFK1, the gene PFK2, the gene PFK26, the gene PFK26, the gene PFK27, the gene GND1, the gene GND2, the gene PMI40, the gene ZWF1, the gene GFA1, the gene GLG1, the gene GLG2, the gene INM1, the gene INM2, the gene GLK1, the gene HXK1, the gene HXK2, the gene GAL10, the gene GAL7, the gene YHL012W, the gene UGP1, the gene GSY1, the gene GSY2, the gene DIE2, the gene ALG8, the gene ATG26, the gene SUC2, the gene MAL32, the gene MAL12, the gene YJL216C, and/or, the gene YGR287C, and/or, FEN1, and/or, FKS1, and/or, GSC2, and/or, TPS1 (12, 15, 16, 24, 26, 30, 31, 34, 36, 38, 39, 41, 51, 56, 57, 59-61, 64, 66, 74, 76, 83, 84, 89, 90, 95, 99).

The latter engineered organism can, for example, be used to produce fucosylated sugar derivates such as fucosyllactose, and more specifically α-1,2-fucosyllactose, α-1,3-fucosyllactose, α-1,4-fucosyllactose, α-1,6-fucosyllactose as specialty products with specific fucosyltransferases originating from for example but not limited to Helicobacter pylori, Bacteroides sp., Homo sapiens, Mus musculus, Bos taurus and, Dictyostelium discoideum. In addition, said engineered organism can be used to produce chitosans by a chitine synthase and chitine deacetylase or to produce myo-inositol by introducing inositol-1-phosphate synthase in combination with inositol monophosphatase.

Specific examples of genes which convert said activated saccharide into a specialty product are genes coding for an epimerase, a transferase, a reductase, a (pyro)phosphorylase, a (de)carboxylase, a dehydratase, a permease, a synthase and/or an isomerase. Therefore the present invention relates to a metabolically engineered organism as indicated above wherein the genes which convert said activated saccharide into a specialty product code for an epimerase, transferase, reductase, dehydrogenase, oxidase, pyrophosphorylase, (de)carboxylase, dehydratase, permease, synthase and/or isomerase. The present invention even more specifically relates to the latter metabolically engineered organisms wherein said epimerase is UDP-galactose-4-epimerase or UDP-N-acetylglucosamine epimerase, and/or, wherein said transferase is a glycosyltransferase, a sialyltransferase or a sulfotransferase.

The invention further relates to a metabolically engineered organism as indicated above wherein said specialty product is a monosaccharide, a disaccharide, a trisaccharide, a tetrasaccharide, a pentasaccharide, an oligosaccharide, a polysaccharide, a nucleoside, an O-glycoside, an S-glycoside, an N-glycoside, a C-glycoside, a glycoprotein, a glycolipid or an activated carbohydrate such as but not limited to myo-inositol, L-arabinose, Scyllo-inosose, Glycosylphosphatidylinositol, Lacto-N-biose, Lacto-N-tetraose, Lactosamine, Fucosylated galactosyloligosaccharides (GOS), L-Fucose, N-Ac glucosamine, Sialic acid, Sialyllactose, chitosan, chitin, 1,2-fucosyllactose, 1,3-fucosyllactose, 1,4-fucosyllactose, 1,6-fucosyllactose, galactinol, stachyose, globotriose, galactose(beta1-4)rhamnose, sophorose, cellobiose, UDP-glucose and sophorolipids.

The present invention further relates to a method to produce a specialty product as described above comprising:
i) metabolically engineering a microorganism as described above, and
ii) cultivating said genetically engineered microorganism, and
iii) extracting and purifying said specialty product.

It is clear that any methodology known in the art to cultivate micro-organisms, and, to extract and purify specialty products from said cultivation can be employed in the present invention.

In addition, the present invention relates to the usage of a 2-fucosyltransferase originating from Dictyostelium discoideum and having an amino acid sequence given by SEQ ID NO: 1, or, a fragment thereof having 2-fucosyltransferase activity, or, a variant thereof having a sequence identity of at least 75% and having 2-fucosyltransferase activity to produce 2-fucosyllactose (α1,2-fucosyllactose). A specific fragment having 2-fucosyltransferase activity as indicated above is given by SEQ ID NO: 4.

Also the usage of a nucleic acid encoding for a 2-fucosyltransferase as indicated above, and specifically wherein said nucleic acid is given by SEQ ID NO: 2 or SEQ ID NO: 3 (which both encode for SEQ ID NO: 1), to produce fucosyllactose is part of present invention. Nucleic acids encoding for SEQ ID NO: 4 are given by SEQ ID NO: 5 and SEQ ID NO: 6 and are also part of the present invention.

The term ‘fragment’ refers to a protein (or peptide or polypeptide) containing fewer amino acids than the amino acid sequence as depicted by SEQ ID NO: 1 and that retains said 2-fucosyltransferase activity. Such fragment can-for example-be a protein with a deletion of 10% or less of the total number of amino acids at the C-and/or N-terminus or can correspond to SEQ ID NO: 4. The term “variant” refers to a protein having at least 75% sequence identity, preferably having at least 76-85% sequence identity, more preferably having at least 86-90% sequence identity or most preferably having at least 91, 92, 93, 94, 95, 96, 97, 98 or 99% sequence identity with SEQ ID NO: 1 or with a fragment thereof, and, that encodes for a protein retaining said 2-fucosyltransferase activity.

Hence, orthologues, or genes in other genera and species (than Dictyostellium discoideum the from which SEQ ID NO: 1 is derived) with at least 75% identity at amino acid level, and having the described function are part of the present invention. The percentage of amino acid sequence identity is determined by alignment of the two sequences and identification of the number of positions with identical amino acids divided by the number of amino acids in the shorter of the sequences×100. The latter ‘variant’ may also differ from the protein as depicted by SEQ ID NO: 1 only in conservative substitutions and/or modifications, such that the ability of the protein to have 2-fucosyltransferase activity is retained. A “conservative substitution” is one in which an amino acid is substituted for another amino acid that has similar properties, such that one skilled in the art of protein chemistry would expect the nature of the protein to be substantially unchanged. In general, the following groups of amino acids represent conservative changes: (1) ala, pro, gly, glu, asp, gln, asn, ser, thr; (2) cys, ser, tyr, thr; (3) val, ile, leu, met, ala, phe; (4) lys, arg, his; and (5) phe, tyr, trp, his.

Variants may also (or alternatively) be proteins as described herein modified by, for example, by the deletion or addition of amino acids that have minimal influence on the 2-fucosyltransferase activity as defined above, secondary structure and hydropathic nature of the enzyme.

The following specific sequences, as indicated above, are part of the present invention:

SEQ ID NO: 1: the complete amino acid sequence of the 2-
fucosyltransferase of Dictyostellium discoideum:
MNDSPIISVVLPFLIKDNDDKSLNYQGINNLIISIDSIIEQTFKEWELILVDDGSNNEILE
QLLSKRYSTDNRIKFIINKENKGIVKSLNDAILNHCSPTSKYIARMDSDDISHPTRLQSQL
KYLQSNETIDILGCPIKMFNNNKLIEILNNNNNNNNINNNVKELINIINNEESFKFIQHPD
KDILMWSMFFNCCIVHPSVIFKRSIFTIEHCYEENNQFPFIEDYLFWLKSLIMKGLNISNI
QSSTPLLYLRKHNNSISFKNIEKQKDSTANASCYYLNILFKRFNIDSEIIQNSSLSMKEII
QFFQLSPSSLSKINNISIELFEFAFKYLELIEKSCTKQQPNYSNSIKDAANEKMGELVSLC
LSNYPNNQKSSLLWEKWLSRNPTSQLLSLLSNLNVKSSTTIINNNINNNNNNNNNNNNNNN
NNNNNNNNNNNNNSILNFISGINSNKINTPKSNNNKFKENGIRIICFSKDRAFQLKEYLRT
FFKYLKNDDNGNDKFEIIVDVLFTYSNEKFKNSYQLVIESFPQVNFIKEENFTDQLINLVQ
KTNKLEYVMFSVDDILYYNEFNLKEYCLSLNSEPLALGFYMKLNKNITYCHTCNQDITIPL
NSNTISRTENNFKYLKWNRNDNDCKKDWNYPWDLCSTIYRCNDIDSIINGIVKYYGIRNGI
NHPNRFEFNGNRPIIQKQIYQNKPYCLCLSDHYSPMSVVTINRVQDVYDNPIYDQTLSLDD
LDQLLYSNKSLNDEKYKENSLSLNFKSVHIGELFIS
SEQ ID NO: 2: the codon optimized nucleotide sequence encoding
SEQ ID NO: 1 for expression in E. Coli:
ATGAACGATAGCCCGATTATTAGCGTTGTTCTGCCGTTTCTGATCAAAGATAACGATGATA
AAAGCCTGAACTACCAGGGCATTAACAACCTGATTATTAGCATCGATAGCATCATCGAGCA
GACCTTTAAAGAATGGGAACTGATTCTGGTTGATGATGGCAGCAATAACGAAATTCTGGAA
CAGCTGCTGAGCAAACGTTATAGCACCGATAACCGCATCAAATTTATTATTAATAAAGAAA
ATAAAGGCATTGTGAAAAGCCTGAATGATGCCATTCTGAATCATTGTAGCCCGACCAGCAA
ATATATTGCACGTATGGATAGCGACGATATTAGCCATCCGACCCGTCTGCAGAGCCAGCTG
AAATATCTGCAGAGCAATGAAACCATTGATATTCTGGGTTGCCCGATCAAAATGTTTAATA
ATAATAAACTGATTGAAATTCTGAATAATAATAACAATAACAACAATATTAATAATAATGT
GAAAGAACTGATTAATATTATTAATAATGAAGAAAGCTTTAAATTTATTCAGCATCCGGAT
AAAGATATTCTGATGTGGTCCATGTTCTTCAATTGCTGTATTGTTCATCCGAGCGTGATTT
TTAAACGCAGCATTTTTACCATCGAGCACTGCTATGAAGAGAATAATCAGTTTCCGTTCAT
CGAGGATTACCTGTTTTGGCTGAAATCCCTGATTATGAAAGGCCTGAACATTAGCAATATC
CAGAGCAGCACACCGCTGCTGTATCTGCGTAAACATAATAACAGCATTAGCTTTAAAAATA
TTGAAAAACAGAAAGATAGCACCGCCAATGCCAGCTGTTATTATCTGAACATTCTGTTCAA
ACGCTTTAACATCGACAGCGAAATTATTCAGAATAGCAGCCTGAGCATGAAAGAAATCATC
CAGTTTTTTCAGCTGAGCCCGAGCAGCCTGTCCAAAATTAATAACATTAGCATCGAACTGT
TTGAATTTGCCTTTAAATATCTGGAACTGATCGAGAAAAGCTGTACCAAACAGCAGCCGAA
TTATAGCAACAGCATTAAAGATGCAGCCAACGAAAAAATGGGTGAACTGGTTAGCCTGTGT
CTGAGCAATTATCCGAATAATCAGAAAAGCAGTCTGCTGTGGGAAAAATGGCTGAGCCGTA
ATCCGACCAGCCAGCTGCTGAGTCTGCTGAGCAATCTGAATGTTAAAAGCAGCACCACCAT
TATTAATAACAATATTAACAACAACAATAATAATAACAACAATAATAACAATAACAATAAC
AATAATAACAACAACAACAATAATAATAATAACAACAACAGCATTCTGAATTTTATTAGCG
GCATTAATAGCAATAAAATTAATACCCCGAAAAGCAACAATAACAAATTTAAAGAGAATGG
CATTCGCATTATTTGCTTCAGCAAAGATCGTGCATTCCAGCTGAAAGAATATCTGCGCACC
TTCTTCAAATATCTGAAAAATGATGATAATGGCAATGATAAATTTGAAATTATTGTGGATG
TGCTGTTTACCTATAGCAATGAAAAATTCAAAAATAGCTATCAGCTGGTGATCGAAAGCTT
TCCGCAGGTTAACTTTATTAAAGAAGAAAACTTTACCGATCAGCTGATTAACCTGGTGCAG
AAAACCAACAAACTGGAATATGTGATGTTCAGCGTGGATGATATCCTGTATTACAACGAGT
TCAATCTGAAAGAGTATTGCCTGAGCCTGAATAGCGAACCGCTGGCACTGGGTTTTTATAT
GAAACTGAATAAAAATATTACCTATTGCCATACCTGCAACCAGGATATTACCATTCCGCTG
AATAGCAATACCATTAGCCGCACCGAAAATAACTTTAAATACCTGAAATGGAATCGCAACG
ATAATGATTGCAAAAAAGACTGGAACTATCCGTGGGATCTGTGTAGCACCATTTATCGTTG
CAACGACATTGACAGCATCATTAATGGTATTGTGAAATATTATGGTATTCGCAACGGCATT
AATCATCCGAATCGCTTTGAATTTAATGGCAACCGTCCGATTATTCAGAAACAAATCTACC
AGAACAAACCGTATTGTCTGTGCCTGAGCGATCATTATTCACCGATGAGCGTTGTTACCAT
TAATCGTGTTCAGGATGTGTATGATAACCCGATTTATGATCAGACCCTGAGCCTGGATGAT
CTGGATCAACTGCTGTATAGCAATAAATCCCTGAACGATGAAAAATATAAAGAAAACAGCC
TGAGTCTGAACTTCAAAAGCGTTCATATTGGCGAACTGTTCATCAGCTAA
SEQ ID NO: 3: the native nucleotide sequence encoding SEQ ID
NO: 1: the 2-fucosyltransferase of Dictyostelium discoideum:
ATGAATGATTCACCAATAATAAGTGTAGTTTTACCTTTTTTAATAAAGGACAATGACGATA
AATCATTAAATTACCAAGGAATAAATAATTTAATAATATCAATAGATAGCATTATTGAACA
AACTTTTAAAGAATGGGAATTAATTTTAGTTGATGATGGATCAAATAATGAAATTTTGGAG
CAATTACTTTCAAAAAGATATAGTACAGATAATAGAATTAAATTCATAATAAATAAAGAGA
ATAAAGGTATTGTTAAAAGTTTAAATGATGCAATTTTAAATCATTGTTCACCAACTTCAAA
ATATATTGCTCGTATGGATTCAGATGATATTTCTCATCCAACAAGATTACAATCTCAACTT
AAATATCTTCAATCAAATGAAACAATTGATATATTAGGTTGTCCAATTAAAATGTTTAATA
ATAATAAATTAATTGAAATTTTAAATAATAATAATAATAATAATAATATTAATAATAATGT
GAAAGAGTTAATTAATATAATTAATAATGAAGAATCTTTTAAATTTATTCAACATCCTGAT
AAAGATATTTTAATGTGGTCAATGTTTTTCAATTGTTGTATTGTTCACCCTTCTGTAATAT
TTAAAAGATCGATATTCACTATTGAACATTGTTATGAAGAAAACAACCAATTTCCATTCAT
TGAAGATTACTTATTTTGGTTAAAATCCTTAATAATGAAAGGTTTAAATATTTCAAATATC
CAATCATCAACACCATTACTATATTTAAGAAAACATAATAACTCTATATCTTTTAAAAATA
TTGAAAAACAAAAAGATTCCACTGCTAATGCATCTTGTTATTATCTAAATATACTTTTTAA
AAGATTTAATATTGATTCTGAAATTATTCAAAATTCTTCACTCTCAATGAAAGAAATTATT
CAGTTCTTTCAACTTTCACCATCATCTTTATCAAAAATCAATAATATTTCAATTGAATTAT
TTGAATTTGCATTTAAATATCTAGAATTAATTGAAAAATCATGTACAAAACAACAACCAAA
CTATTCAAACAGTATAAAAGATGCAGCAAATGAAAAAATGGGTGAATTAGTATCTTTATGT
TTATCAAATTATCCAAATAATCAAAAATCATCATTACTTTGGGAAAAATGGTTATCAAGAA
ATCCAACCTCACAATTACTATCACTTTTATCAAATTTAAATGTAAAATCTTCAACTACTAT
AATTAATAATAATATTAATAATAATAATAATAATAATAATAATAATAATAATAATAATAAT
AATAATAATAATAATAATAATAATAATAATAATAATAATTCAATTTTAAATTTTATATCTG
GCATTAATAGTAATAAAATAAATACTCCAAAATCTAATAATAATAAATTTAAAGAAAATGG
AATTAGAATAATTTGTTTCTCAAAAGATAGAGCATTTCAATTAAAAGAATATCTTAGAACA
TTTTTTAAATATTTAAAAAATGATGATAATGGAAATGATAAATTTGAAATTATTGTTGATG
TATTATTTACATATTCAAATGAGAAATTCAAAAACTCTTATCAATTAGTTATTGAAAGTTT
TCCACAAGTTAATTTTATTAAAGAAGAGAATTTCACTGATCAATTAATTAATTTAGTTCAA
AAAACAAATAAACTTGAATATGTCATGTTTTCAGTTGATGATATTCTTTATTATAATGAAT
TCAATCTCAAAGAATATTGTTTATCTTTGAATAGTGAGCCATTGGCATTAGGTTTCTATAT
GAAGTTAAATAAAAATATTACCTATTGTCATACTTGTAATCAAGATATAACAATACCATTA
AATTCAAATACTATTAGTAGAACAGAGAATAATTTTAAATATTTAAAATGGAATAGAAATG
ATAATGATTGTAAAAAGGATTGGAATTATCCATGGGATTTATGTTCAACCATTTATAGATG
TAATGATATTGATTCAATCATTAATGGTATAGTTAAATATTATGGAATTAGAAATGGTATT
AATCATCCAAATAGATTCGAATTCAATGGTAATAGACCAATCATTCAAAAGCAAATCTATC
AAAATAAACCCTACTGTTTATGTTTATCAGATCACTATTCTCCAATGTCTGTTGTAACTAT
TAATAGAGTTCAAGATGTCTATGATAATCCAATTTATGACCAAACCCTATCTTTAGATGAT
TTAGATCAATTACTTTATTCAAACAAATCATTAAATGATGAAAAATATAAAGAAAATAGTT
TATCTTTAAATTTTAAAAGTGTTCATATTGGTGAACTTTTTATTTCTTAA
SEQ ID NO: 4: the amino acid sequence of a fragment of SEQ ID
NO: 1 having fucosyltransferase activity:
MSILNFISGINSNKINTPKSNNNKFKENGIRIICFSKDRAFQLKEYLRTFFKYLKNDDNGN
DKFEIIVDVLFTYSNEKFKNSYQLVIESFPQVNFIKEENFTDQLINLVQKTNKLEYVMFSV
DDILYYNEFNLKEYCLSLNSEPLALGFYMKLNKNITYCHTCNQDITIPLNSNTISRTENNF
KYLKWNRNDNDCKKDWNYPWDLCSTIYRCNDIDSIINGIVKYYGIRNGINHPNRFEFNGNR
PIIQKQIYQNKPYCLCLSDHYSPMSVVTINRVQDVYDNPIYDQTLSLDDLDQLLYSNKSLN
DEKYKENSLSLNFKSVHIGELFIS
SEQ ID NO: 5: the codon optimized nucleic acid sequence
encoding SEQ ID NO: 4 with an ATG added:
ATGAGCATTCTGAATTTTATTAGCGGCATTAATAGCAATAAAATTAATACCCCGAAAAGCA
ACAATAACAAATTTAAAGAGAATGGCATTCGCATTATTTGCTTCAGCAAAGATCGTGCATT
CCAGCTGAAAGAATATCTGCGCACCTTCTTCAAATATCTGAAAAATGATGATAATGGCAAT
GATAAATTTGAAATTATTGTGGATGTGCTGTTTACCTATAGCAATGAAAAATTCAAAAATA
GCTATCAGCTGGTGATCGAAAGCTTTCCGCAGGTTAACTTTATTAAAGAAGAAAACTTTAC
CGATCAGCTGATTAACCTGGTGCAGAAAACCAACAAACTGGAATATGTGATGTTCAGCGTG
GATGATATCCTGTATTACAACGAGTTCAATCTGAAAGAGTATTGCCTGAGCCTGAATAGCG
AACCGCTGGCACTGGGTTTTTATATGAAACTGAATAAAAATATTACCTATTGCCATACCTG
CAACCAGGATATTACCATTCCGCTGAATAGCAATACCATTAGCCGCACCGAAAATAACTTT
AAATACCTGAAATGGAATCGCAACGATAATGATTGCAAAAAAGACTGGAACTATCCGTGGG
ATCTGTGTAGCACCATTTATCGTTGCAACGACATTGACAGCATCATTAATGGTATTGTGAA
ATATTATGGTATTCGCAACGGCATTAATCATCCGAATCGCTTTGAATTTAATGGCAACCGT
CCGATTATTCAGAAACAAATCTACCAGAACAAACCGTATTGTCTGTGCCTGAGCGATCATT
ATTCACCGATGAGCGTTGTTACCATTAATCGTGTTCAGGATGTGTATGATAACCCGATTTA
TGATCAGACCCTGAGCCTGGATGATCTGGATCAACTGCTGTATAGCAATAAATCCCTGAAC
GATGAAAAATATAAAGAAAACAGCCTGAGTCTGAACTTCAAAAGCGTTCATATTGGCGAAC
TGTTCATCAGCTAA
SEQ ID NO: 6: the native nucleic acid sequence encoding SEQ ID
NO: 4:
TCAATTTTAAATTTTATATCTGGCATTAATAGTAATAAAATAAATACTCCAAAATCTAATA
ATAATAAATTTAAAGAAAATGGAATTAGAATAATTTGTTTCTCAAAAGATAGAGCATTTCA
ATTAAAAGAATATCTTAGAACATTTTTTAAATATTTAAAAAATGATGATAATGGAAATGAT
AAATTTGAAATTATTGTTGATGTATTATTTACATATTCAAATGAGAAATTCAAAAACTCTT
ATCAATTAGTTATTGAAAGTTTTCCACAAGTTAATTTTATTAAAGAAGAGAATTTCACTGA
TCAATTAATTAATTTAGTTCAAAAAACAAATAAACTTGAATATGTCATGTTTTCAGTTGAT
GATATTCTTTATTATAATGAATTCAATCTCAAAGAATATTGTTTATCTTTGAATAGTGAGC
CATTGGCATTAGGTTTCTATATGAAGTTAAATAAAAATATTACCTATTGTCATACTTGTAA
TCAAGATATAACAATACCATTAAATTCAAATACTATTAGTAGAACAGAGAATAATTTTAAA
TATTTAAAATGGAATAGAAATGATAATGATTGTAAAAAGGATTGGAATTATCCATGGGATT
TATGTTCAACCATTTATAGATGTAATGATATTGATTCAATCATTAATGGTATAGTTAAATA
TTATGGAATTAGAAATGGTATTAATCATCCAAATAGATTCGAATTCAATGGTAATAGACCA
ATCATTCAAAAGCAAATCTATCAAAATAAACCCTACTGTTTATGTTTATCAGATCACTATT
CTCCAATGTCTGTTGTAACTATTAATAGAGTTCAAGATGTCTATGATAATCCAATTTATGA
CCAAACCCTATCTTTAGATGATTTAGATCAATTACTTTATTCAAACAAATCATTAAATGAT
GAAAAATATAAAGAAAATAGTTTATCTTTAAATTTTAAAAGTGTTCATATTGGTGAACTTT
TTATTTCTTAA

The present invention is hereby following illustrated by specific working examples.

### EXAMPLES

### Example 1. Materials and Methods

### Media

The Luria Broth (LB) medium consisted of 1% tryptone peptone (Difco, Erembodegem, Belgium), 0.5% yeast extract (Difco) and 0.5% sodium chloride (VWR, Leuven, Belgium). The medium for the shake flasks experiments contained 2.00 g/l NH4Cl, 5.00 g/l (NH4)2SO4, 2.993 g/l KH2PO4, 7.315 g/l K2HPO4, 8.372 g/l MOPS, 0.5 g/l NaCl, 0.5 g/l MgSO4·7H20, 14.26 g/l sucrose or another carbon source when specified in the examples, 1 ml/l vitamin solution, 100 μl/l molybdate solution, and 1 ml/l selenium solution. The medium was set to a pH of 7 with 1M KOH.

Vitamin solution consisted of 3.6 g/l FeCl2·4H20, 5 g/l CaCl2·2H2O, 1.3 g/l MnCl2· 2H2O, 0.38 g/l CuCl2·2H2O, 0.5 g/l CoCl2·6H2O, 0.94 g/l ZnCl2, 0.0311 g/l H3BO4, 0.4 g/l Na2EDTA·2H2O and 1.01 g/l thiamine·HCl. The molybdate solution contained 0.967 g/l Na2MoO4·2H2O. The selenium solution contained 42 g/l SeO2.

The minimal medium for fermentations contained 6.75 g/l NH4Cl, 1.25 g/l (NH4)2SO4, 1.15 g/l KH2PO4 (low phosphate medium) or 2.93 g/l KH2PO4 and 7.31 g/l KH2PO4 (high phosphate medium), 0.5 g/l NaCl, 0.5 g/l MgSO4·7H2O, 14.26 g/l sucrose, 1 ml/l vitamin solution, 100 μl/l molybdate solution, and 1 ml/l selenium solution with the same composition as described above.

Complex medium was sterilized by autoclaving (121° C., 21′) and minimal medium by filtration (0.22 μm Sartorius). If necessary the medium was made selective by adding an antibiotic (ampicilin, chloramphenicol, kanamycin).

### Cultivation Conditions

A preculture, from a single colony on a LB-plate, in 5 ml LB medium was incubated during 8 hours at 37° C. on an orbital shaker at 200 rpm. From this culture, 2 ml was transferred to 100 ml minimal medium in a 500 ml shake flask and incubated for 16 hours at 37° C. on an orbital shaker at 200 rpm. 4% inoculum was used in a 2|Biostat B Plus culture vessel with 1.5|working volume (Sartorius Stedim Biotech, Melsungen, Germany). The culture conditions were: 37° C., stirring at 800 rpm, and a gas flow rate of 1.5 l/min. Aerobic conditions were maintained by sparging with air. The pH was maintained at 7 with 0.5 M H₂SO4 and 4 M KOH. The exhaust gas was cooled down to 4° C. by an exhaust cooler (Frigomix 1000, Sartorius Stedim Biotech, Melsungen, Germany). 10% solution of silicone antifoaming agent (BDH 331512K, VWR Int Ltd., Poole, England) was added when foaming raised during the fermentation (approximately 10 μl). The off-gas was measured with an EL3020 off-gas analyser (ABB Automation GmbH, 60488 Frankfurt am Main, Germany). All data was logged with the Sartorius MFCS/win v3.0 system (Sartorius Stedim Biotech, Melsungen, Germany).

All strains were cultivated at least twice and the given standard deviations on yields and rates are based on at least 10 data points taken during the repeated experiments.

### Sampling

The bioreactor contains in its interior a harvest pipe (BD Spinal Needle, 1.2×152 mm (BDMedical Systems, Franklin Lakes, NJ-USA) connected to a reactor port, linked outside to a Masterflex-14 tubing (Cole-Parmer, Antwerpen, Belgium) followed by a harvest port with a septum for sampling. The other side of this harvest port is connected back to the reactor vessel with a Masterflex-16 tubing. This system is referred to as rapid sampling loop. During sampling, reactor broth is pumped around in the sampling loop. It has been estimated that, at a flow rate of 150 ml/min, the reactor broth needs 0.04 s to reach the harvest port and 3.2 s to re-enter the reactor. At a pO2 level of 50%, there is around 3 mg/l of oxygen in the liquid at 37° C. The pO2 level should never go below 20% to avoid micro-aerobic conditions. Thus 1.8 mg/l of oxygen may be consumed during transit through the harvesting loop. Assuming an oxygen uptake rate of 0.4 g oxygen/g biomass/h (the maximal oxygen uptake rate found at μₘₐₓ), this gives for 5 g/l biomass, an oxygen uptake rate of 2 g/l/h or 0.56 mg/l/s, which multiplied by 3.2 s (residence time in the loop) gives 1.8 mg/l oxygen consumption.

In order to quench the metabolism of cells during the sampling, reactor broth was sucked through the harvest port in a syringe filled with 62 g stainless steel beads precooled at −20° C., to cool down 5 ml broth immediately to 4° C. Sampling was immediately followed by cold centrifugation (15000 g, 5 min, 4° C.). During the batch experiments, samples for OD₆₀₀ₙₘ, CDW, and extracellular metabolites were taken each hour using the rapid sampling loop and the cold stainless bead sampling method. When exponential growth was reached, the sampling frequency was increased to every 20 to 30 minutes.

### Broth Sampling

Using a rapid sampling, which was coupled to the fermentor, samples of 1 ml broth were withdrawn from the fermentor within 0.5 s. Samples were withdrawn directly into tubes containing 5 ml of quenching solution precooled at −40° C. that were immediately mixed after sampling by vortexing. The exact sample sizes were quantified gravimetrically by weighing the tubes before and after sampling.

### Filtrate Sampling

Samples of extracellular culture fluid were obtained with syringe filtration (pore size 0.45 μm, cellulose acetate) at room temperature without beads-Direct filtration of the broth sample After removal of the cells, the obtained filtrate or supernatant was immediately mixed with 5 ml of quenching solution to process these samples in the same way as the broth samples. Also in this case, the exact amount of sample obtained was quantified gravimetrically.

### Quenching Procedure

The quenching solution used was a 60% (v/v) aqueous methanol. After quenching of broth samples in the quenching solution, precooled at −40° C., the sample/quenching solution mixture was centrifuged for 5 min at 8000 g in a cooled centrifuge (−20° C.) using a rotor that was precooled at −40° C. After decanting, the supernatant (QS) was stored at −40° C. until extraction. Subsequently, the cell pellets were resuspended in 5 ml of −40° C. quenching solution and again centrifuged. Also, this second supernatant (WS) was stored at −40° C. until extraction. For measurement of metabolites in total broth as well as in the culture filtrate, the same quenching procedure was applied; however, the quenched total broth mixtures (B) or quenched culture filtrates (F) were not centrifuged, but after thorough vortexing, 500 μl of these mixtures was withdrawn for metabolite extraction.

### Metabolite Extraction Procedure

Extraction of metabolites from the cell pellets as well as from the 500-μl samples from the quenched total broth was performed with the hot ethanol method [34]. Metabolites were extracted in 75% boiling ethanol (3 min, 90° C.). After cooling the thus obtained extracts were evaporated to dryness in a RapidVap (Labconco Corporation, Kansas, Missouri, USA) during 110 min under vacuum. After resuspension of each residue in 500 μL of H2O, cell debris was removed by centrifugation during 5 min at 5000 g. After decanting the supernatants were stored at −80° C. until further analysis.

### Analytical Methods

Cell density of the culture was frequently monitored by measuring optical density at 600 nm (Uvikom 922 spectrophotometer, BRS, Brussel, Belgium). Cell dry weight was obtained by centrifugation (15 min, 5000 g, GSA rotor, Sorvall RC-5B, Goffin Meyvis, Kapellen, Belgium) of 20 g reactor broth in pre-dried and weighted falcons. The pellets were subsequently washed once with 20 ml physiological solution (9 g/l NaCl) and dried at 70° C. to a constant weight. To be able to convert OD₆₀₀ₙₘ measurements to biomass concentrations, a correlation curve of the OD₆₀₀ₙₘ to the biomass concentration was made. The concentrations of glucose and organic acids were determined on a Varian Prostar HPLC system (Varian, Sint-Katelijne-Waver, Belgium), using an Aminex HPX-87H column (Bio-Rad, Eke, Belgium) heated at 65° C., equipped with a 1 cm precolumn, using 5 mM H2SO4 (0.6 ml/min) as mobile phase. A dual-wave UV-VIS (210 nm and 265 nm) detector (Varian Prostar 325) and a differential refractive index detector (Merck LaChrom L-7490, Merck, Leuven, Belgium) was used for peak detection. By dividing the absorptions of the peaks in both 265 and 210 nm, the peaks could be identified. The division results in a constant value, typical for a certain compound (formula of Beer-Lambert).

### Carbohydrate Measurements

Glucose, fructose, sucrose and glucose-1-phosphate were measured by HPLC with a Hypercarb column (100×4,6 mm; 5 μm particle size) and were detected with an ELSD detector or mass spectrometer (Antonio et al., 2007; Nielsen et al., 2006). The LOQ of sucrose and G1P were 30 and 20 mg/l, respectively. All samples were diluted within the linear range of the detector, which is between the LOQ and approximately 100 mg/l of the metabolite. When multiple phosphorylated and nucleotide sugars were present in the broth, an adaptation of the method of Bucholz et al was applied (11). In this case a gradient of milliQ water (A) and 20 mM ammonium acetate (B) was used to separate the analytes. The gradient started at 100% A with a flow of 1 ml/min and changed to 100% B at 1ml/min over 10 minutes. The eluens composition of 100% B was then held for 4 minutes at 1ml/min and then changed to 100% A at 1 ml/min over 1 minute, after which the flow was increased to 1.2 ml/min and held for 3 minutes to reduce the equilibration time of the column. After these three minutes the flow was reduced again in 2 minutes to 1 ml/min. All analytes were detected with either an ELSD detector or mass spectrometer.

For the analysis of mono-, di-, and oligo-saccharides a Prevail Carbohydrate ES (5μ; 250×4.6 mm) column was used with a gradient of 100% aceton (A), 100% acetonitril (B) and 100% water (C). The gradient is initiated at 20% A, 60% B and 20% C. This is changed over 15 minutes to 15% A, 45% B and 40% C and then changed back to 20% A, 60% B and 20% C within 1 minute.

The column is then equilibrated at its initial conditions for 6 minutes. All analytes were either measured with ELSD or mass spectrometer.

### Measurement of Cell dry Weight

From a broth sample, 4×10 g was transferred to centrifuge tubes, the cells were spun down (5000g, 4° C., 5 min), and the cells were washed twice with 0.9% NaCl solution. The centrifuge tubes containing the cell pellets were dried in an oven at 70° C. for 48 h until constant weight. The cell dry weight was obtained gravimetrically; the tubes were cooled in a desiccator prior to weighing.

### Sophorose Polysaccharide Measurement

To determine the amount of sophorose polysaccharide that was produced by a mutant strain in which the heterologous tts gene (50) was expressed, a 100 ml culture of this mutant and of the wild type strain at approximately OD 6 was centrifuged (5500 rpm, 4° C., 5 minutes, Heraus Biofuge stratos). 80 ml of the supernatant was then precipitated with 2 volumes of cold ethanol (100% at −20° C.) en stored overnight at 6° C. The precipitate was separated from the supernatant by centrifugation (5500 rpm, 4° C., 5 min, Hereaus Biofuge stratos) en resuspended in 25 ml distilled water (88). 2 ml of this polysaccharide solution was then hydrolyzed in pyrex boriumsilicate tubes (26×100 mm) at 105° C. with 2.25 M HCl (final concentration) for 4 h. To neutralize the solution for glucose measurement, equimolar amounts of NaOH was added to the solution after incubation and cooling. The amount of glucose in the solution was determined with an YSI biochemistry analyser (YSI (UK) Ltd.).

Strains and Plasmids Used for Dictyostellium discoideum α1,2-fucosyltransferase Characterization

A codon optimized α1,2-fucosyltransferase originating from Dictyostellium discoideum was expressed heterologously in E. coli which has the genotype ΔlacZΔglgCΔmanAΔCA on a plasmid which was constructed as described by Aerts et al. (1). CA indicates all genes in the gene cluster that codes for the colanic acid biosynthetic pathway described by Stevenson et al. (86)

### Enzyme Isolation Methodology

The strains were grown in LB (10 g/l tryptone, 5 g/l yeast extract and 10 g/l NaCL) in an overnight culture (100 ml in 500 ml shake flask) at 37° C. and 200 rpm. The cells were harvested by centrifugation (15 minutes at 7500 rpm and 4° C.). This pellet was resuspended in 5ml PBS buffer and sonicated 3 times for 4 minutes on ice water (cycle 50%, intensity 3). The cell debris was centrifuged again 15 minutes at 7500 rpm and 4° C. The supernatant was used as crude cell extract.

### Protein Determination

Protein content of the enzyme extract was measured with the Pierce BCA Protein Assay Kit (Thermo) as specified in the product manual.

### Plasmid Construction for the Expression of Heterologous and Homologous Genes

Plasmid which was constructed as described by Aerts et al. (1).

### Genetic Methods

Plasmids were maintained in the host E. coli DH5α (F⁻, φ80dlacZΔM15, Δ(lacZYA-argF)U169,deoR, recA1, endA1, hsdR17(rk⁻, mk⁺), phoA, supE44, λ⁻, thi-1, gyrA96, relA1).

Plasmids. pKD46 (Red helper plasmid, Ampicillin resistance), pKD3 (contains an FRT-flanked chloramphenicol resistance (cat) gene), pKD4 (contains an FRT-flanked kanamycin resistance (kan) gene), and pCP20 (expresses FLP recombinase activity) plasmids were obtained from Prof. Dr. J-P Hernalsteens (Vrije Universiteit Brussel, Belgium). The plasmid pBluescript (Fermentas, St. Leon-Rot, Germany) was used to construct the derivates of pKD3 and pKD4with a promoter library, or with alleles carrying a point mutation.

Mutations. The mutations consisted in gene disruption (knock-out, KO), replacement of an endogenous promoter by an artificial promoter (knock-in, KI), respectively. They were introduced using the concept of Datsenko and Wanner (19).

Transformants carrying a Red helper plasmid were grown in 10 ml LB media with ampicillin (100 mg/l) and L-arabinose (10 mM) at 30° C. to an OD₆₀₀ₙₘ of 0.6. The cells were made electro competent by washing them with 50 ml of ice-cold water, a first time, and with 1 ml ice-cold water, a second time. Then, the cells were resuspended in 50 μl of ice-cold water. Electroporation was done with 50 μl of cells and 10-100 ng of linear double-stranded-DNA product by using a Gene Pulser™ (BioRad) (600 Ω, 25 μFD, and 250 volts).

After electroporation, cells were added to 1 ml LB media incubated 1 h at 37° C., and finally spread onto LB-agar containing 25 mg/l of chloramphenicol or 50 mg/l of kanamycin to select antibiotic resistant transformants. The selected mutants were verified by PCR with primers upstream and downstream of the modified region and were grown in LB-agar at 42° C. for the loss of the helper plasmid. The mutants were tested for ampicillin sensitivity.

Elimination of the antibiotic resistance gene

The selected mutants (chloramphenicol or kanamycin resistant) were transformed with pCP20plasmid, which is an ampicillin and chloramphenicol resistant plasmid that shows temperature-sensitive replication and thermal induction of FLP synthesis. The ampicillin-resistant transformants were selected at 30° C., after which a few were colony purified in LB at 42° C. and then tested for loss of all antibiotic resistances and of the FLP helper plasmid. The gene knock-outs and knock-ins are checked with control primers and sequenced.

The primers used to construct the various Knock-out and knock-in mutants are listed in Table 1.

TABLE 1
Primers used for the construction of the gene knock outs

| gene | Fw-P1-H1 | Rv-P2-H2 |
|---|---|---|
| lacZ | CATAATGGATTTCCTTACGCGAAATAC | GTATGTTGTGTGGAATTGTGAGCGGAT |
| | GGGCAGACATGGCCTGCCCGGTTATTA | AACAATTTCACACAGGAAACAGCTcat |
| | gtgtaggctggagctgcttc | atgaatatcctccttag |
| glgC | agaccgccggttttaagcagcgggaac | gtctggcagggacctgcacacggattg |
| | atctctgaacatacatgtaaaacctgc | tgtgtgttccagagatgataaaaaagg |
| | agtgtaggctggagctgcttc | agttagtccatatgaatatcctcctta |
| | | g |
| agp | CATATTTCTGTCACACTCTTTAGTGAT | TAAAAACGTTTAACCAGCGACTCCCCC |
| | TGATAACAAAAGAGGTGCCAGGAgtgt | GCTTCTCGCGGGGGAGTTTTCTGcata |
| | aggctggagctgcttc | tgaatatcctccttag |
| pgi | GGCGCTACAATCTTCCAAAGTCACAAT | GGTTGCCGGATGCGGCGTGAACGCCTT |
| | TCTCAAAATCAGAAGAGTATTGCgtgt | ATCCGGCCTACATATCGACGATGcata |
| | aggctggagctgcttc | tgaatatcctccttag |
| pfkA | GACTTCCGGCAACAGATTTCATTTTGC | GCTTCTGTCATCGGTTTCAGGGTAAAG |
| | ATTCCAAAGTTCAGAGGTAGTCgtgta | GAATCTGCCTTTTTCCGAAATCcatat |
| | ggctggagctgcttc | gaatatcctccttag |
| pfkB | CACTTTCCGCTGATTCGGTGCCAGACT | GTTGCCGACAGGTTGGTGATGATTCCC |
| | GAAATCAGCCTATAGGAGGAAATGgtg | CCAATGCTGGGGGAATGTTTTTGcata |
| | taggctggagctgcttc | tgaatatcctccttag |
| pgm via | TGAGAAGGTTTGCGGAACTATCTAAAA | CATACGTAAAAAAGGGCGATCTTGCGA |
| D&W | CGTTGCAGACAAAGGACAAAGCAgtgt | CCGCCCTTTTTTTATTAAATGTGTcat |
| | aggctggagctgcttc | atgaatatcctccttag |
| pgm::kan | TGAGAAGGTTTGCGGAACTATCTAAAA | CATACGTAAAAAAGGGCGATCTTGCGA |
| | CGTTGCAGACAAAGGACAAAGCAACGA | CCGCCCTTTTTTTATTAAATGTGTAGA |
| | AAGGCTCAGTCGAAAG | ACTCCAGCATGAGATCC |
| pgm::GFP | TGAGAAGGTTTGCGGAACTATCTAAAA | CATACGTAAAAAAGGGCGATCTTGCGA |
| | CGTTGCAGACAAAGGACAAAGCAgtgt | CCGCCCTTTTTTTATTAAATGTGTCAT |
| | aggctggagctgcttc | CCGTCAGGATGGCCTTC |
| ptsG | gccacgcgtgagaacgtaaaaaaagca | Cacctgtaaaaaaggcagccatctggc |
| | cccatactcaggagcactctcaattgt | tgccttagtctccccaacgtcttacgg |
| | gtaggctggagctgcttc | acatatgaatatcctccttag |
| glk | CGAGAAGGCCCGGATTGTCATGGACGA | CCAGGTATTTACAGTGTGAGAAAGAAT |
| | TGAGATACACCGGAATATCATGGgtgt | TATTTTGACTTTAGCGGAGCAGTTGAA |
| | aggctggagctgcttc | GAcatatgaatatcctccttag |
| malPQ | ATATCCAGCCAGTCTTCCGGCTGTAGT | GCTTTAAGTGGTTGAGATCACATTTCC |
| | CCTAACAGAGCACTGTTACTGTCagca | TTGCTCATCCCCGCAACTCCTCCcata |
| | ttacacgtcttgagcg | tgaatatcctccttag |
| MP KI | ATATCCAGCCAGTCTTCCGGCTGTAGT | CAACGGCCATTTTTTGCACTTAGATAC |
| | CCTAACAGAGCACTGTTACTGTCGTAA | AGATTTTCTGCGCTGTATTGCATTGCC |
| | AACGACGGCCAGTG | GGGATCCGATGCATATGG |
| ycjU | TTTTATTTTGCCCTTCAATGGGACCGC | TTCCGTTGAAGGCAACAGTAATTGCGC |
| | TACCAAACATCAGGAGGATGAATGAAA | CCCGGTTAAGCCCGCGCCGATCCcata |
| | Cagcattacacgtcttgagcg | tgaatatcctccttag |
| CA via | GTAGCATTGTTCCTAAGTATGACTCCA | TTCACGCCGCATCCGGCAAGCAAACCA |
| D&W | TTTTTCCAGGAATGGTCGCAAATCgtg | GCTCATAAGCCGGGAGAACAACCcata |
| | taggctggagctgcttc | tgaatatcctccttag |
| CA via | TTCACGCCGCATCCGGCAAGCAAACCA | GTAGCATTGTTCCTAAGTATGACTCCA |
| sacB | GCTCATAAGCCGGGAGAACAACCccgc | TTTTTCCAGGAATGGTCGCAAATCagc |
| | ttacagacaagctgtg | catgacccgggaattac |
| wcaJ via | ATCGCCGACCACTTCGCGCCGCTGATG | GGATCTTCCCTTACCCCACTGCGGGTA |
| sacB | GTTTTTTCACGTAAGCTCATATCccgc | AGGGGCTAATAACAGGAACAACGagcc |
| | ttacagacaagctgtg | atgacccgggaattac |
| wcaJ via | GGGGGCCCCCGGGGGTATGAGCTTACG | GGGCCCGGGCCCGGGCGTTGTTCCTGT |
| sacB and | TGAAAAAACCATCAG | TATTAGCCCCTTACCC |
| fusion | | |
| PCR | | |
| wcaJ via | ATCGCCGACCACTTCGCGCCGCTGATG | GGATCTTCCCTTACCCCACTGCGGGTA |
| D&W | GTTTTTTCACGTAAGCTCATATCgtgt | AGGGGCTAATAACAGGAACAACGcata |
| | aggctggagctgcttc | tgaatatcctccttag |
| wcaJ via | TTTTGATATCGAACCAGACGCTCCATT | TCTATGGTGCAACGCTTTTCAGATATC |
| D&W_2 | CGCGGATGTACTCAAGGTCGAACgtgt | ACCATCATGTTTGCCGGACTATGcata |
| | aggctggagctgcttc | tgaatatcctccttag |
| galET- | TAGCCAAATGCGTTGGCAAACAGAGAT | CGGTTCGACGCATGCAGGCATGAAACC |
| H1-P22- | TGTGTTTTTTCTTTCAGACTCATCTTT | GCGTCTTTTTTCAGATAAAAAGCcata |
| RBS | GTTTCCTCCGAATTCG | tgaatatcctccttag |
| galET | ACCAATCAAATTCACGCGGCCAGGCGC | GTCGGTAGTGCTGACCTTGCCGGAGGC |
| extended | CTGAATGGTGTGAGTGGCAGGGTAGCC | GGCCTTAGCACCCTCTCCGGCCAACGG |
| homology | AAATGCGTTGGCAAAC | TTCGACGCATGCAGGC |

### Example 2. Engineering and Usage of Base Strain 1 (Carbon Source: Sucrose; Converted into Glucose-1-Phosphate and Fructose by Sucrose Phosphorylase)—Screening of Different Sucrose Phosphorylases

An important requirement for the success of ‘base strain 1’ is the existence of a potent sucrose phosphorylase. However, though E. coli has a putative sucrose phosphorylase (SP), it does not grow on a minimal medium with sucrose as sole carbon source. Therefore, 6 sucrose phosphorylases from a variety of microbial sources were screened (Table 2).

To this end, 6 transformants of the wild type E. coli were constructed each carrying a plasmid [pCX-promoter-SP] encoding for one of the sucrose phosporylase (SP) listed in Table 2. The performance of these strains was evaluated in shake flasks, using sucrose as sole carbon source. The wild type strain (WT) was incorporated in the experimental design as a control (μ_{WT}=0).

TABLE 2
Screened sucrose phosphorylases

| Source sucrose phosphorylase | Abbreviation |
|---|---|
| Bifidobacterium adolescentis | BA |
| Lactobacillus acidophilus | LA |
| Streptococcus mutans | SM |
| Leuconostoc mesenteroides B742 | LM B742 |
| Leuconostoc mesenteroides B1149 | LM B1149 |
| Leuconostoc mesenteroides B1355 | LM B1355 |

In this screening experiment, the growth rate of the various transformants was monitored and linked to the performance of the sucrose phosphorylases. According to this reasoning the best growing strain does posses the best performing sucrose phosphorylase.

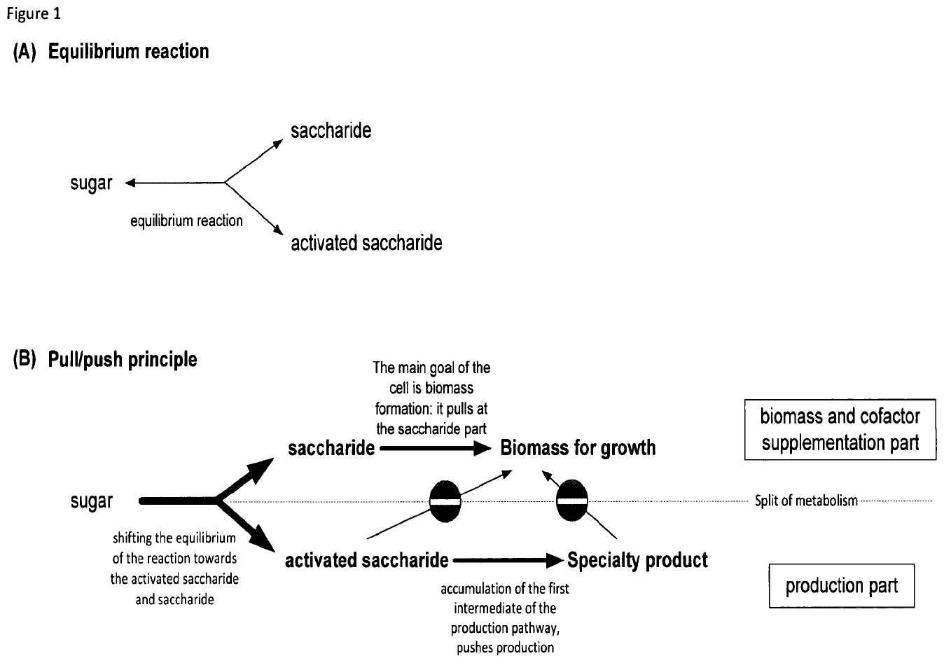

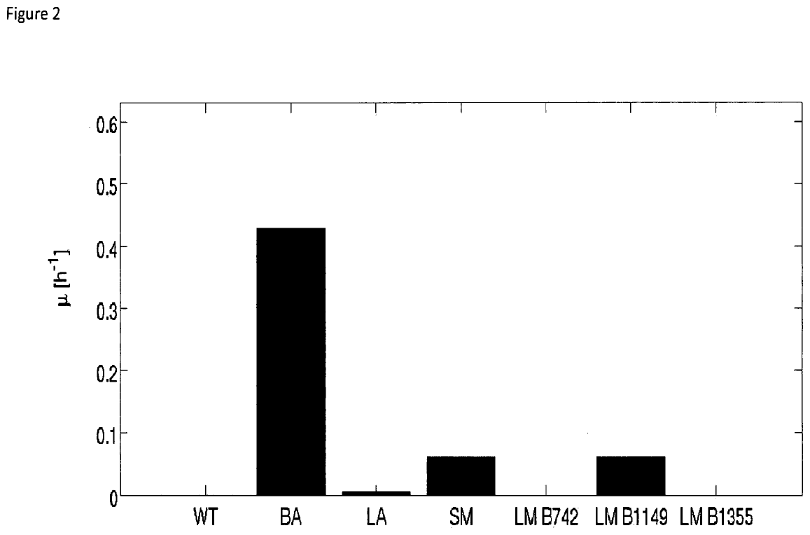

The growth rate of the various transformants is depicted in FIG. 2, the principle applied on for the production of a specialty carbohydrate is depicted in FIG. 1: (a) A normal equilibrium reaction, which occurs in the current production technologies (b) pull/push principle: the equilibrium is shifted towards the saccharide and activated saccharide. The main goal of a cell is to grow, hence it pulls, in this figure at the saccharide to form biomass. Due to this pulling effect, the activated saccharide will accumulate in the cell and pushes the production pathway.

### Example 3. Characterization of the Sucrose Phosphorylase of Bifidobacterium adolescentis

Various artificial constitutive promoters have been inserted to evaluate the influence of the promoter strength on the growth rate. To this end, a weak, medium strength, and strong promoter from a promoter library available at The Centre of Expertise-Industrial Biotechnology and Biocatalysis (Ghent University) were introduced. The medium strength promoter, which yielded the highest growth rate, was finally retained.

The affinity constant and the maximal growth rate of the E. coli strain carrying a plasmid encoding for the sucrose phosphorylase of Bifidobacterium adolescentis were determined. To this end, batch and chemostat experiments were run. The influence of the phosphate concentration on these parameters was checked as well (Table 3).

The kinetic properties of the engineered strain are listed in Table 3. It is clear that the kinetic properties of the engineered strain are adequate in view of future industrial applications.

TABLE 3
Growth characteristics of an E. coli carrying the Bifidobacterium
adolescentis sucrose phosphorylase.
High PO₄³⁻ = 64 mM; low PO₄³⁻ = 8.5 mM.

| | High PO₄³⁻ | Low PO₄³⁻ |
|---|---|---|
| μₘₐₓ (h⁻¹) | 0.5 | 0.46 |
| K_{S} (mg/L) | <10 | +/−10 |

### Example 4. Engineering Strategy for an Increased Supply of α Glucose-1-Phosphate

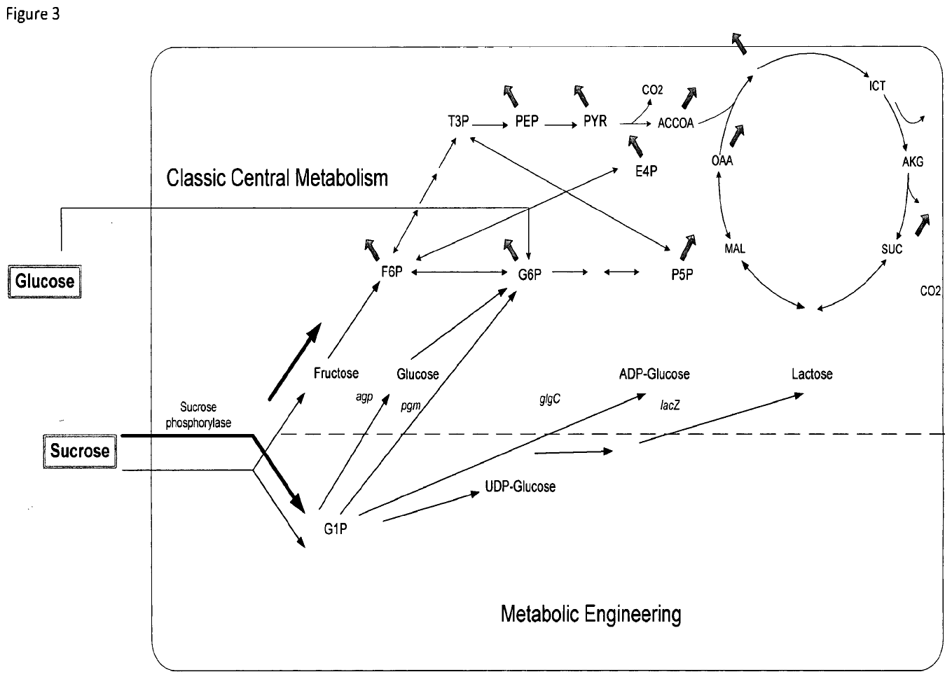

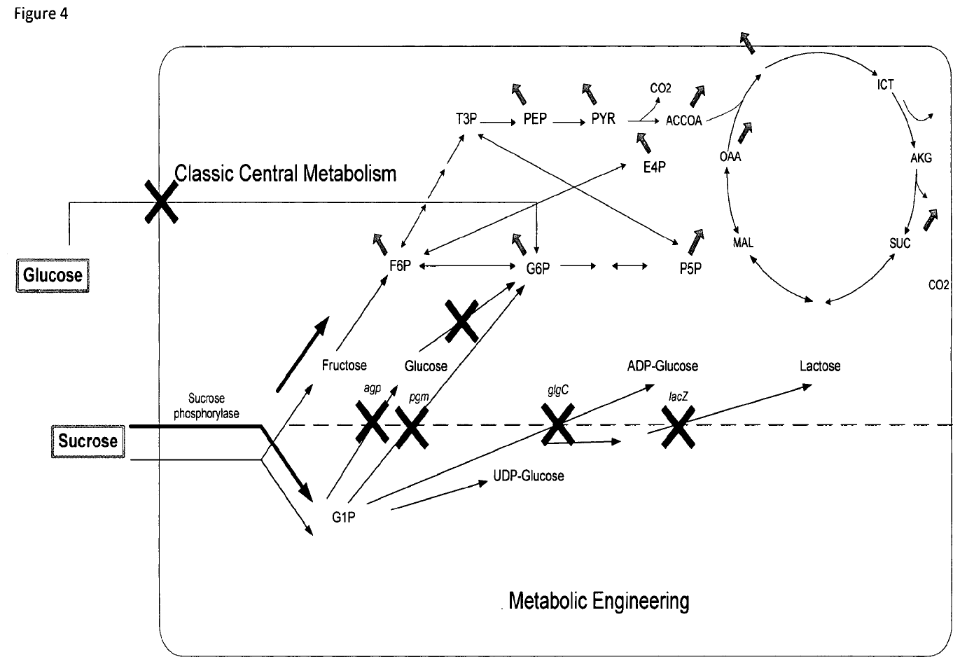

To validate the rational of the engineering strategy it is important to demonstrate an increased pool of αglucose-1-phosphate in the mutant strain (FIG. 3), compared to the αglucose-1-phosphate pool in the wild type (FIG. 4). In ‘Base strain 1’ the microbial metabolism is split into two disconnected parts because the main reactions able to convert a glucose-1-phosphate to biomass production were eliminated. One part of the metabolism converts the fructose moiety into biomass and numerous bio-catalytic enzymes (classic central metabolism). The other part converts the αglucose-1-phosphate moiety of sucrose.

The α glucose-1-phosphate concentration was determined both for the wild type and some engineered strains. To this end, batch experiments were performed, using sucrose as sole carbon source.

### The α Glucose-1-Phosphate Pool: Comparing the Wild Type and the Plug Strain

To evaluate the potential of the envisaged metabolic engineering strategy, the αglucose-1-phosphate pool was determined in:
the wild type E. coli MG1655 grown on glucose,
E. coli MG1655 p22BaSP grown on sucrose,
E. coli MG1655 ΔglgC Δpgm ΔlacZ p22BaSP grown on sucrose

The size of this pool is of major importance because the metabolically engineered pathways of the various specialty carbohydrates to be produced all use αglucose-1-phosphate as prime precursor. Hence, the larger the αglucose-1-phosphate pool, the more precursors that is available for the production of the various specialty carbohydrates.

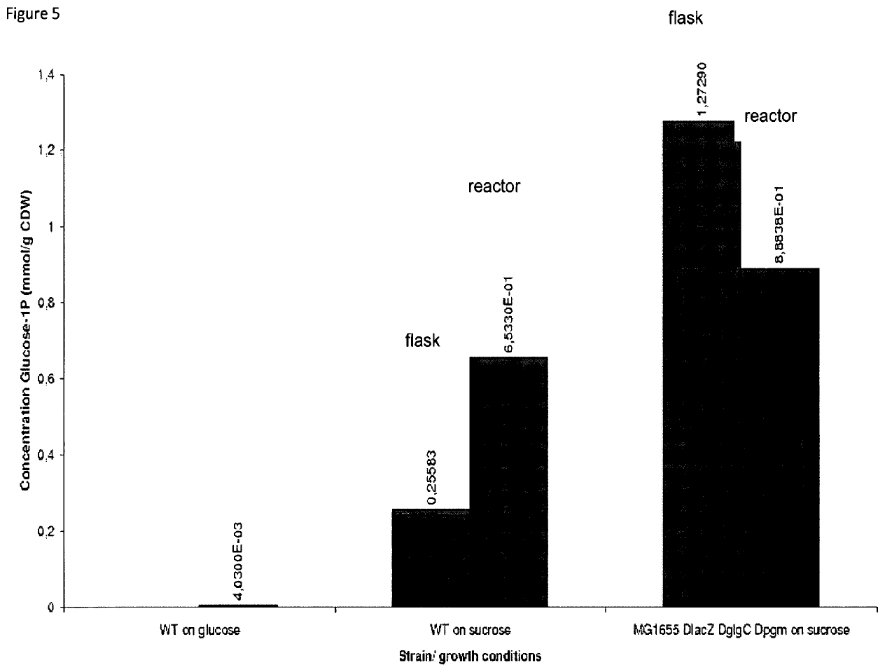

The shake flasks results are depicted in FIG. 5. The intracellular glucose-1-phosphate concentration is 4.03 10⁻³ mmol/gcDW, 0.26 mmol/gcDW, and 1.27 mmol/gcDW, respectively. A>20000% increase in the G1P pool is thus already achieved. This increased pool enables the efficient production of a variety of specialty carbohydrates.

In the wild type E. coli MG1655 strain glucose-1-phosphate is a precursor of cell wall related components, glycogen, etc. A limited flow of carbon, typically coming from αglucose-6-phosphate, suffices to supply the cell with sufficient αglucose-1-phosphate to produce these minor biomass fractions. Hence, the αglucose-1-phosphate pool is of limited size (4.03 10⁻³ mmol/gcDW).

This is in contrast with the proposed strategy to use sucrose as carbon source. Compared to the wild type E. coli MG1655 strain an increased glucose-1-phosphate pool has been shown in the mutant strains that contain a potent sucrose phosphorylase that efficiently splits the inexpensive sugar sucrose into fructose and αglucose-1-phosphate.

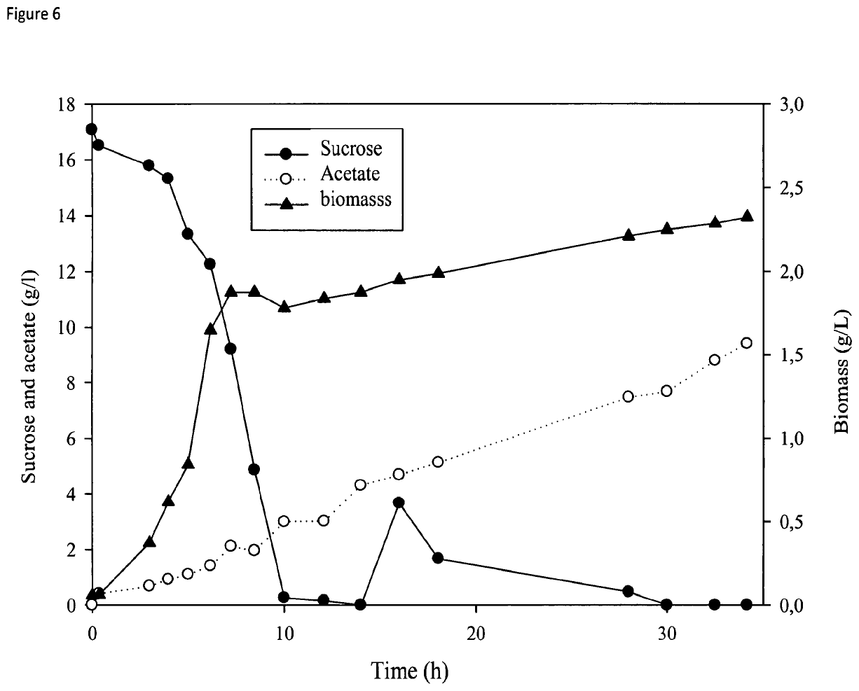

The results obtained in a 1.5 L batch reactor are depicted in FIG. 6. The intracellular αglucose-1-phosphate concentration is 4.03 10⁻³ mmol/gcDW, 0.65 mmol/gcDW, and 0.89 mmol/gcDW, respectively.

Production of αglucose-1-Phosphate by ΔpgmΔlacZΔglgC (3KO) P22-BaSP on Buffered LB Medium at Reactor Scale

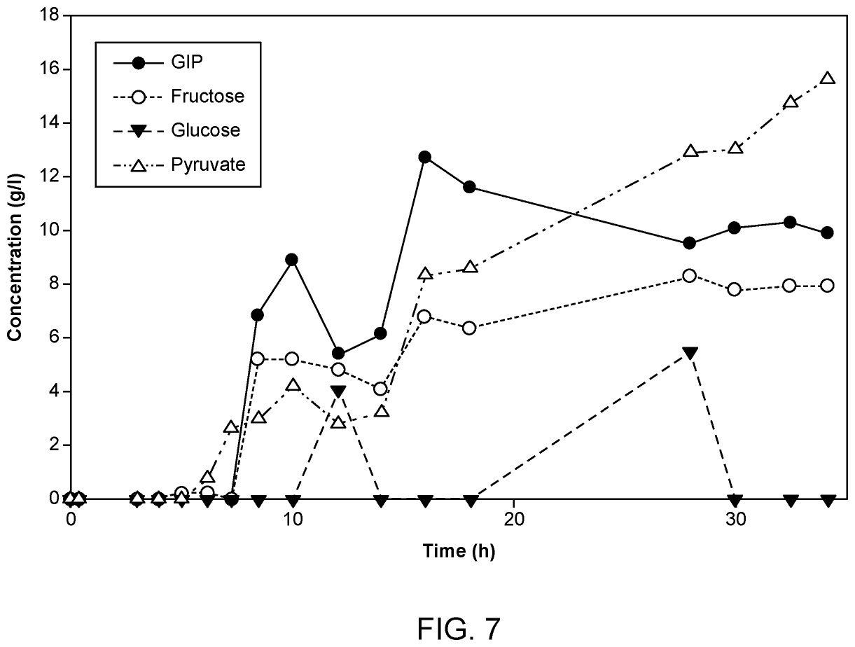

The ability of ΔpgmΔlacZΔglgC P22-BaSP to produce αglucose-1-phosphate was verified. To this end, a culture with buffered LB medium with about 15 g/L sucrose was run. At about 15 h a concentrated sucrose solution, containing phosphate was added. In FIG. 7 and FIG. 6 the concentration of the most important (by)products are depicted. The maximal growth rate during the batch phase is about 0,552 h-1.

During the batch phase per mole of sucrose that is degraded 0.74 mole of glucose-1-phosphate is generated. Ideally, 1 mole of αglucose-1-phosphate can be generated. However, the 3KO studied still contains genes whose products are able to convert αglucose-1-phosphate, e.g., agp.

From the moment all sucrose is consumed, the concentration of glucose-1-phosphate decreases and the concentration of glucose increases which is due to the activity of Agp.

Subsequently at about 15 h additional sucrose is added, which is again converted to glucose-1-phosphate and which accumulates in the medium. Fructose accumulates as well in the medium, which indicates that the cell has limited means to further metabolize this compound (0.64 mole of fructose per mole of sucrose).

In a subsequent experiment, sucrose and phosphate were added on regular time intervals during the course of the fermentation. A maximum αglucose-1-phosphate concentration of about 57 g/L was achieved.

### Example 5. Inactivation of the Gene Coding for Phosphoglucomutase

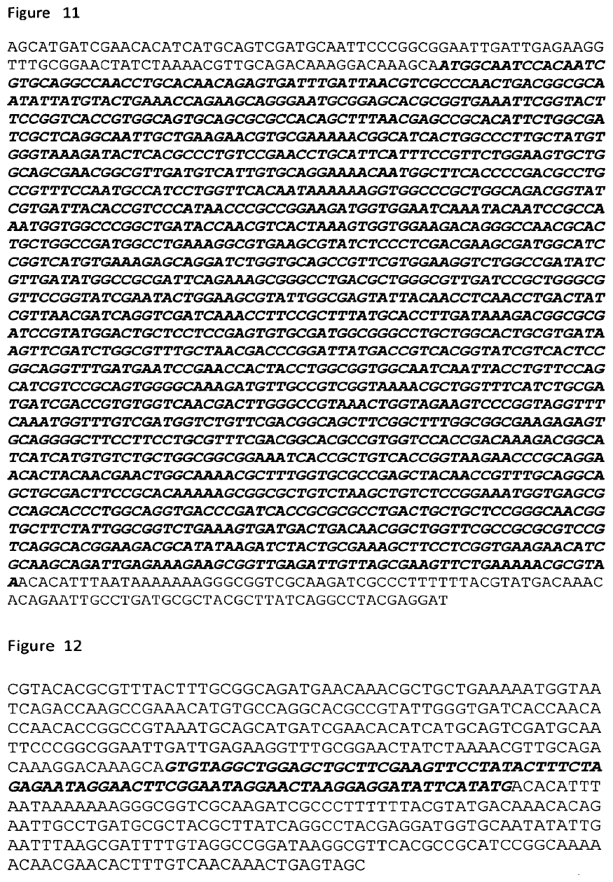

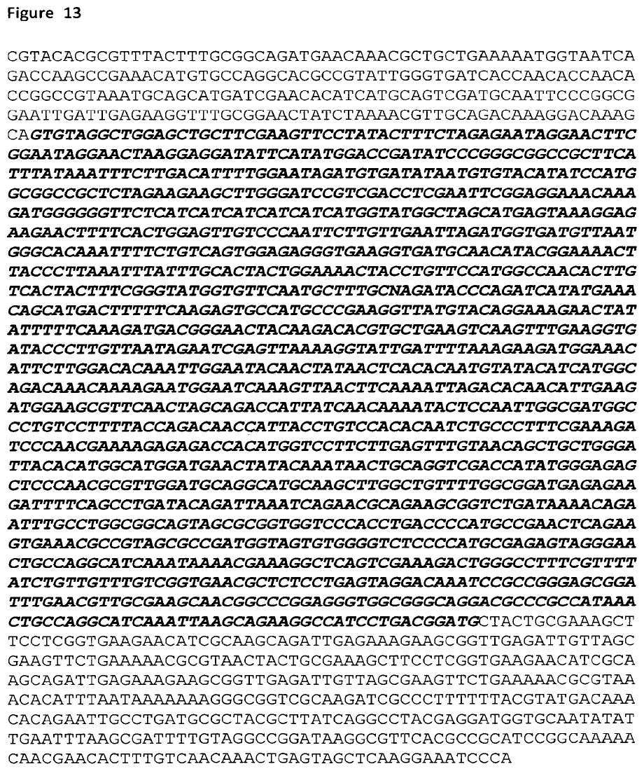

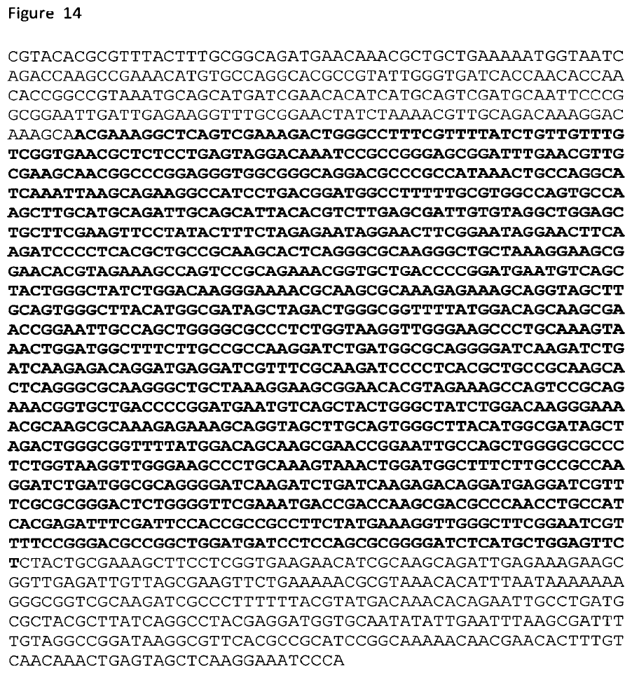

To split the metabolism according to example 1-4 the gene coding for phosphoglucomutase has to be knocked out. Via the classical methodology described by Datsenko and Wanner (19) a knock out results into a chromosomal scar of approximately 84 base pairs. The strains in which this gene was deleted in this manner seem to grow on a complex medium but, to our surprise, did not grow on a minimal medium as described in the materials and methods section. However, the strain did grow on a minimal medium when the kanamycine cassette was left behind. Apparently the removal of the original sequence at this chromosomal location seemed to interfere with growth on a minimal medium but the replacement of this specific sequence (pgm gene), coding for phosphoglucomutase, by a sequence with a similar length did not. This fact was validated by replacing the pgm gene with a part of the GFP gene which has exactly the same size as the pgm gene. This resulted also in a mutant strain that could grow on a minimal medium. The sequences of these strains at the chromosomal location of pgm are shown in FIG. 11, FIG. 12, FIG. 13, and FIG. 14.

### Example 6. Cellobiose Production in E. coli

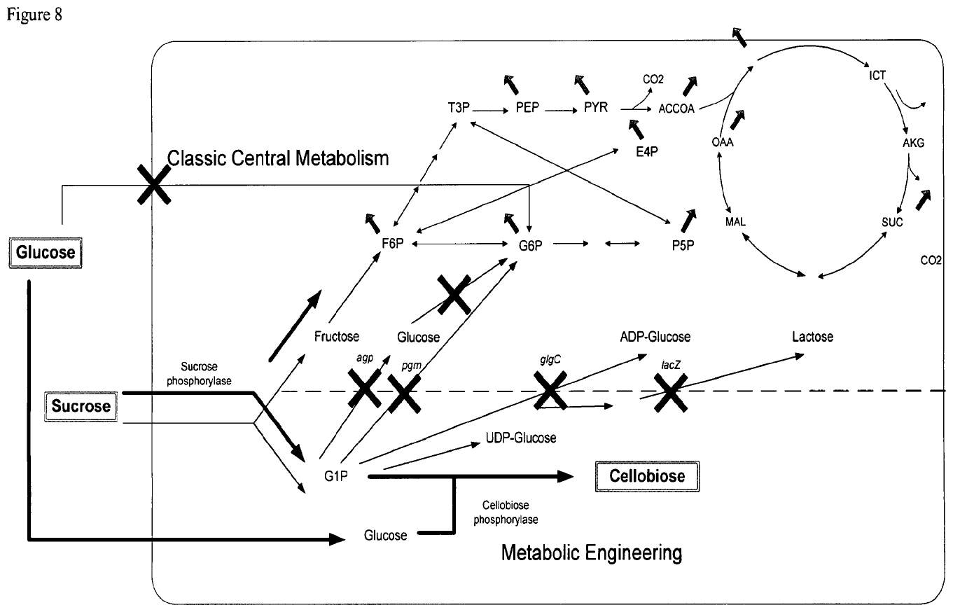

Cellobiose producing strains have been constructed starting from ‘Base strain 1’ (FIG. 8). To this end a plasmid containing both a sucrose phosphorylase (Bifidobacterium adolescentis) and cellobiose phosphorylase (Cellulomonas uda) have been inserted in the wild type, in E. coli MG1655 ΔΔglgC Δpgm ΔlacZ (3KO), and in E. coli MG1655 Δagp ΔglgC Δpgm ΔlacZ (4KO) (Table 4). Additional genes to be knocked out are glk and ptsG, since both convert glucose into glucose-6-phosphate.

TABLE 4
Cellobiose producing strain

| Knock-out | Reaction |
|---|---|
| lacZ | Glu + Gal ↔ Lactose |
| pgm | G1P ↔ G6P |
| glgC | G1P + ATP + H ↔ ADP-glucose + PPi |
| agp | G1P + H₂O → Glu + Pi |
| ycjM | Suc → G1P + Fruc |
| ptsG | Glu + PEP → G6P + Pyr |
| glk | Glu + ATP → G6P + ADP |
| Knock-in | Reaction |
| Sucrose | Sucrose + Pi→ G1P + Fruc |
| phosphorylase | |
| Cellobiose | G1P + Glu → Cellobiose + Pi |
| phosphorylase | |

### Comparing the Wild Type and the Plug Strain

To evaluate the potential of the envisaged metabolic engineering strategy to produce specialty carbohydrates the production of cellobiose was investigated in various engineered strains:
E. coli MG1655 (WT) P22-BaSP P22-CuCP
E. coli MG1655 ΔglgC Δpgm ΔlacZ (3KO) P2-BaSP P22-CuCP.
E. coli MG1655 ΔglgC Δpgm ΔlacZ Δagp (4KO) P22-BaSP P22-CuCP

To this end shake flask experiments were performed. The medium contained buffered LB medium and sucrose and glucose were added in equal amounts to the shake flasks, so that a final concentration of 1.978 g cellobiose/L was achieved in the shake flask (Table 5). The shake flasks results are depicted in FIG. 5. The (extracellular) concentration of the desired product cellobiose increases with the number of mutations that has been introduced.

TABLE 5
Cellobiose production of various engineered strains

| Strain | Abbreviation | Cellobiose (g/L) |
|---|---|---|
| E. coli MG1655 P22-BaSP | WT P22-BaSPP | 0 |
| P22-CuCP | 22-CuCP | |
| E. coli MG1655 ΔglgC Δpgm | 3KO P22-BaSP | 0.539 |
| ΔlacZ P22-BaSP P22-CuCP | P22-CuCP | |
| E. coli MG1655 ΔglgC Δpgm | 4KO P22-BaSP | 1.978 |
| ΔlacZ Δagp P22-BaSP P22- | P22-CuCP | |
| CuCP | | |

Production of Cellobiose by ΔpgmΔlacZΔglgCΔagp (4KO) P22-BaSP P22-CuCP on Buffered LB Medium at Reactor Scale

The ability of ΔpgmΔlacZΔglgCΔagp P22-BaSP P22-CuCP to produce cellobiose was verified on reactor scale in a preliminary experiment. To this end, a culture with buffered LB medium was run. At about 9 h and on specific time points a solution containing 500 g/L sucrose and 250 g/L glucose was added to the culture.

A conversion efficiency of about 30% (mol cellobiose produced/mol sucrose consumed) was achieved and about 40% of the glucose moiety of sucrose ended up in cellobiose or in glucose-1-phosphate. A titer of about 15 g/L of cellobiose was achieved at the end of the culture.

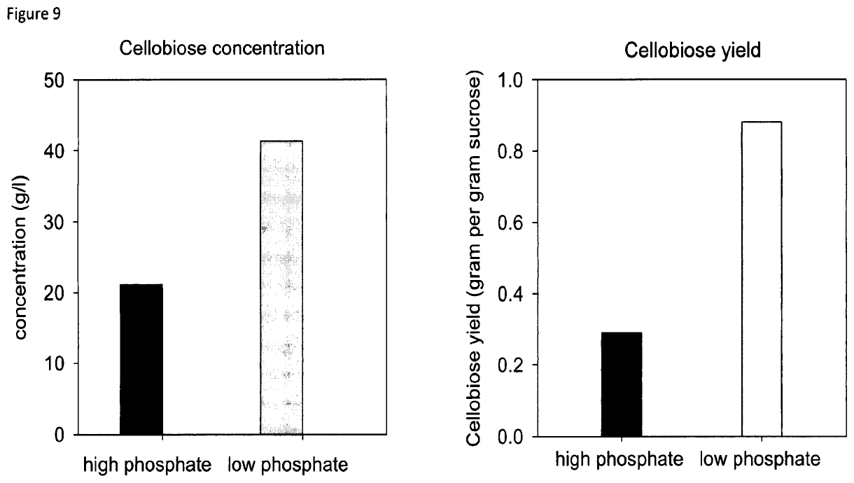

Secondly, the production of cellobiose was verified in a batch culture starting from 70 g/l sucrose with a high concentration of phosphate and low concentration of phosphate. High phosphate indicates a phosphate concentration of 0.2 M phosphate, low phosphate indicates a concentration of 13 mM phosphate. This affected the production significantly. The high concentration of phosphate resulted in a final titer of approximately 20 g/l and a yield of 0.33 g/g, while a low phosphate concentration resulted in a final titer of 42 g/l and a yield on consumed sucrose of 0.84 g/g (FIG. 9).

### Example 7. Engineering Base Strain 2 (Sucrose-Sucrose Synthase) and its uses

By metabolically engineering E. coli a base strain is constructed that is an efficient producer of specialty carbohydrates and their derivatives whose pathway starts from UDP-glucose.

By introducing sucrose synthase (e.g., coming from Solanum tuberosum), sucrose is split into fructose and UDP-glucose. By additionally knocking-out genes coding for UDP-glucose 4 epimerase (galE), UDP-glucose galactose-1-P uridilyltransferase (galT), glucose-1-P uridilyltransferase (galU, galF), 5′-nucleotidase/UDP-sugar hydrolase (ushA), UDP-glucose 6-dehydrogenase (ugd), belonging to the colanic acid operon (ca) a mutant is constructed which accumulates UDP-glucose (Table 6).

TABLE 6
Base strain UDP-Glucose

| Knock-out | Reaction |
|---|---|
| ca | → colanic acid |
| galU | G1P + UTP ↔ UDP-Glc + PPi |
| galF | G1P + UTP ↔ UDP-Glc + PPi |
| galE | UDP-Glc ↔ UDP-Gal |
| galT | UDP-Glc + Gal1P ↔ UDP-Gal + G1P |
| ushA | UDP-sugar + H₂O ↔ uridine-5′- |
| | phosphate + 2H⁺ + an aldose-1-phosphate |
| ugd | 2 NAD + UDP-sugar + H₂O ↔ 2 NADH + |
| | UDP-glucuronate + 3H⁺ |
| Knock-in | Reaction |
| Sucrose synthase | Suc + UDP→ UDP Glu + Fruc |

### Example 8. Expression of Sucrose Synthase in E. coli

The activity of sucrose synthase was determined using an in vitro assay. A sucrose synthase from Solanum tuberosum was heterologously expressed in E. coli BL21. From this culture an enzyme extract was prepared, which was incubated with 500 mM sucrose and 2 mM UDP. The evolution of the amount UDP-Glucose produced is given in Table 7.

TABLE 7
Sucrose synthase enzym assay demonstrating the activity
of cleavage reaction of sucrose synthase (Mixture
sontained 500 mM sucrose and 2 mM UDP)

| Sampling time | UDP-Glucose formed |
|---|---|
| 0 h 10 min | 5 mg/L |
| 1 h 40 min | 64 mg/L |
| 24 h | |

### Example 9. Sophorose Production

Starting from base strain 2 (UDP-Glucose), a strain is constructed which produces large quantities of sophorose, as a polymer of sophorose units. This is achieved by additionally introducing the gene tts from Streptococcus pneumoniae (50). Sophorose units can be generated out of the sophorose polymer in two ways, i.e., via acid hydrolysis or via enzymatic conversion. Such an enzyme can be (over) expressed in the producer strain as well.

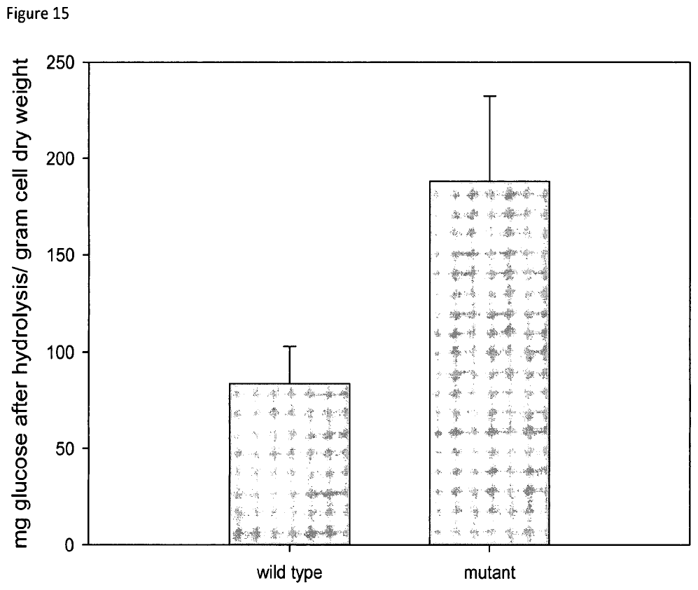

To evaluate the potential of the metabolic engineering strategy the sophorose polymer was determined for E. coli MG1655 P22-BaSP P22-tts, and a 6KO P22-BaSP P22-tts strain (E. coli MG1655 ΔglgC Δpgm ΔlacZ Δagp ΔptsG Δglk) by growing these strains on a minimal medium containing lactose as sole carbon source. The results are depicted in FIG. 15. These results indicate that the mutant strains produce significantly more sophorose polymer in comparison to the wild type strain.

### Example 10. Engineering Base Strain 3 (Lactose-Lactose Phosphorylase) and its uses

By introducing lactose phosphorylase (20), lactose is split into glucose and galactose-1-P. By additionally knocking-out genes coding for agp, galE, galT, lacZ, and the metabolism is split into two disconnected parts. However, all possible combinations of these genes result in increased accumulation of galactose-1-phosphate. Instead of knocking-out these genes, this goal can also be achieved by rendering them defective or by reducing their expression (Table 8).

TABLE 8
Base strain 3 Galactose-1-phosphate (Lactose- lactose phosphorylase)

| Knock-out | Reaction |
|---|---|
| lacZ | Glu + Gal ↔ Lactose |
| galE | UDP-Glc <--> UDP-Gal |
| agp | G1P + H2O → Glu + Pi |
| galT | UDP-Glc + Gal1P <--> UDP-Gal + G1P |
| Knock-in | Reaction |
| Lactose | Lactose + Pi → Gal1P + Glucose |
| phosphorylase | |

### Example 11. Galactose(β1-4)L-Rhamnose Production

Starting from base strain 3, a Gal(β1-4)L-Rha producer is constructed by additionally introducing a gene coding for an (Ga)lacto-N-biose D-galactosyl-(β1-4)-L-rhamnose phosphorylase, which convert Galactose-1-phosphate and rhamnose into Gal(β1-4) L-Rha and phosphate.

A fermentation is performed using lactose as main carbon source yielding quantities of Gal(β1-4)L-Rha. L-rhamnose is added to the medium. Undesired degradation of rhamnose is prevented by knocking out genes involved in the degradation of rhamnose (rhaA, rhaB, rhaC, rhaD).

### Example 12. Engineering Base Strain 4 (Lactose-Lactose Synthase) and its uses

By introducing lactose sythase (71, 72) lactose is split into glucose and UDP-galactose. By additionally knocking-out genes coding for beta-galactosidase (lacZ), UDP-glucose, galactose-1-P uridilyltransferase (galT) UDP-glucose 4 epimerase (galE), 5′-nucleotidase/UDP-sugar hydrolase (ushA), UDP-glucose 6-dehydrogenase (ugd), belonging to the colanic acid operon (ca) a mutant is constructed which accumulates UDP-Galactose (Table 9).

TABLE 9
Base strain 4 UDP-Galactose (Lactose synthase)

| Knock-out | Reaction |
|---|---|
| lacZ | Glu + Gal ↔ Lactose |
| galE | UDP-Glc <--> UDP-Gal |
| galT | UDP-Glc + Gal1P <--> UDP-Gal + G1P |
| ca | → colanic acid |
| ushA | UDP-sugar + H₂O <--> uridine-5′- |
| | phosphate + 2H⁺ + an aldose-1-phosphate |
| ugd | 2 NAD + UDP-sugar + H₂O <--> 2 NADH + |
| | UDP-glucuronate + 3H⁺ |
| Knock-in | Reaction |
| Lactose synthase | Lactose → UDP-Gal + Glu |

### Example 13. Galactinol Production

Starting from base strain 4, a galactinol producer is constructed by additionally introducing a gene coding for an Inositol 3-alpha-galactosyltransferase which catalyzes the conversion:

UDP-galactose+myo-inositol=UDP+O-α-D-galactosyl-(1→3)-1D-myo-inositol

A fermentation is performed using lactose as main carbon source yielding quantities of galactinol in which myo-inositol is added to the medium.

### Example 14. Globotriose Production

Starting from base strain 4, a globotriose producer is constructed by additionally introducing the gene IgtC from Neisseria meningitidis (3) encoding for a α-1,4-Gal transferase, which catalyzes the conversion UDP-Gal+Lactose→UDP+Globotriose. A fermentation is performed using lactose as main carbon source yielding quantities of globotriose.

### Example 15. Engineering Base Strain 5 and Producing Fucosylated Sugars

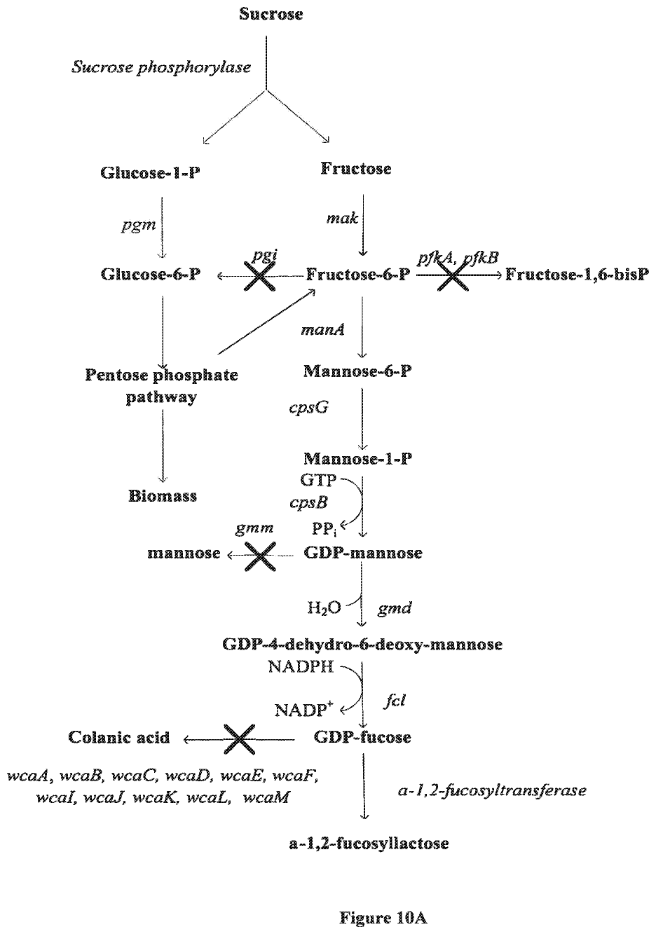

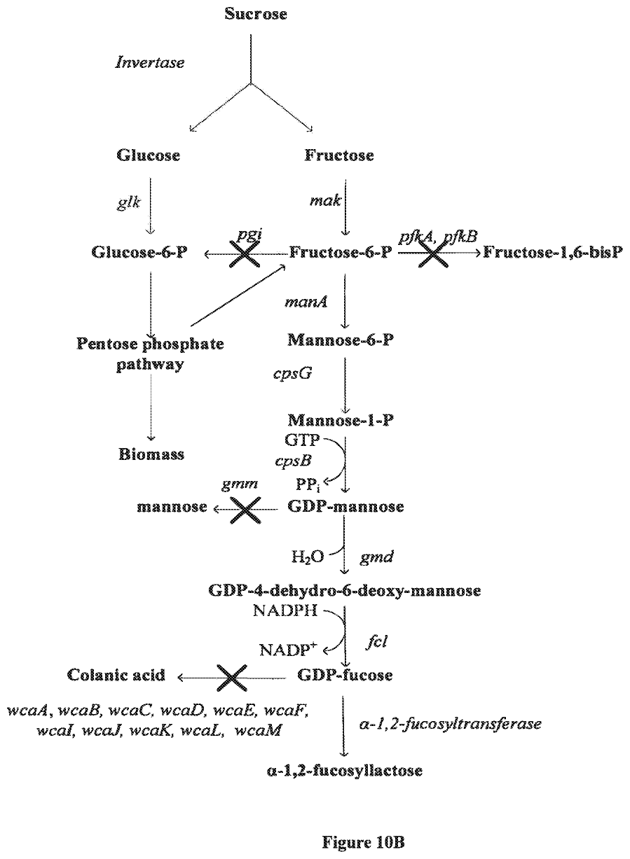

Starting from base strain 5, which accumulates fructose-6-phosphate, (described in Example 20 and Example 28) fucosylated sugar derivates such as fucosyllactose and more specifically 1,2-fucosyllactose can be produced. To this end, the strain is modified so that the cell is forced to produce fructose-6-phosphate which is a precursor of GDP-fucose. Glucose or glucose-1-phosphate (if the starting enzyme is either a sucrase or a sucrose phosphorylase) is then fed to the central carbon metabolism via the pentose phosphate pathway. FIGS. 10A and 10B show the route towards product and biomass and the needed knock outs to achieve this. To avoid loss of fructose-6-phosphate via glycolysis, pfkA, pfkB and pgi are knocked out. To avoid accumulation of pyruvate, the Entner Douderoff route is knocked out (edd and eda).

Because GDP-fucose is an intermediate of the colanic acid biosynthesis pathway, this pathway has to be modified. Genes from the colanic acid operon that can potentially reduce product yield are knocked out. These genes are gmm, wcaA, wcaBi, wcaC, wcaD, wcaE, wcaF, wcal, wcaJ, wcaK, wcaL and/or, wcaM. The genes manA, cpsG, cpsB, gmd and, fcl (coding for Mannose-6-phosphate isomerase, phosphomannomutase, mannose-1-phosphate guanylyltransferase, GDP-mannose 4,6-dehydratase and GDP-fucose synthase, respectively) are enhanced in expression to increase the flux towards GDP-fucose. Either the colanic acid operon is knocked out completely, with the reintroduction of the needed genes that are overexpressed with an artificial promoter from a promoter library, or the operon is modified as such that the genes described above are knocked out one by one and the remaining genes in the operon are enhanced in expression by changing the promoter of the operon with an artificial constitutive promoter (23). Finally GDP-fucose is linked to lactose into α-1,2-fucosyllactose by a α-1,2-fucosyltransferase. The fucosyltransferases tested originate from Helicobacter pylori, Bacteroides sp., Homo sapiens, Mus musculus, Bos taurus and, Dictyostelium discoideum.

### Example 16. Engineering E. coli Base Strain 3 (Galactose-1-P) and its uses

By knocking-out genes coding for (a) phosphatase(s) (agp), UDP-glucose, galactose-1-P uridilyltransferase (galT), UDP-glucose-4-epimerase (galE) a mutant is constructed which accumulates galactose-1-P. By additionally overexpressing genes coding for galactokinase (galK) and/or galactose-1-epimerase (galM) the formation of galactose-1-P is enhanced (Table 10).

TABLE 10
Base strain galactose-1-phosphate

| Knock-out | Reaction |
|---|---|
| galT | Gal1P + UDP-Glucose ↔ UDP- |
| | Galactose + G1P |
| galE | UDP-Glucose ↔ UDP-Galactose |
| agp | Glucose-1-phosphate + H₂O → Pi + |
| | glucose |
| Knock-in | Reaction |
| galK | α-galactose + ATP → Gal1P + ADP |
| galM | β-galactose ↔ α-galactose |

### Example 17. Engineering Base Strain 3 (Galactose-1P) and its uses

By knocking-out genes coding for (a) phosphatase(s) (agp), UDP-glucose, galactose-1-P uridilyltransferase (galT), UDP-glucose-4-epimerase (galE) and by additionally overexpressing genes coding for galactokinase (galK) a mutant is constructed which accumulates galactose-1-P (Table 11).

TABLE 11
E. coli base strain galactose-1-phosphate

| Knock-out | Reaction |
|---|---|
| galT | Gal1P + UDP-Glucose ↔ UDP- |
| | Galactose + G1P |
| galE | UDP-Glucose ↔ UDP-Galactose |
| galK | α-galactose + ATP → Gal1P + ADP |
| galM | β-galactose ↔ α-galactose |
| agp | glucose-1-phosphate + H₂O → Pi + |
| | glucose |
| Knock-in | Reaction |
| galK | α-galactose + ATP → Gal1P + ADP |

To evaluate the potential of the metabolic engineering strategy, the galactose-1-phosphate concentration was determined for the wild type, E. coli MG1655 ΔgalET P22 galK, E. coli MG1655 ΔgalETKM Δagp P22 galK, E. coli MG1655 ΔgalET P22 galK, and E. coli MG1655 ΔgalETKM Δagp P22 galK+orotate (2 g/L) by growing these strains on a minimal medium containing lactose (15 g/L) as main carbon source. The results are depicted in Table 12.

TABLE 12
Galactose-1-P concentration of various E. coli mutants

| strain | Galactose-1-P (mg/L) |
|---|---|
| E. coli MG1655 | Not detectable |
| E. coli MG1655 ΔgalET P22 galK | 15, 73 |
| E. coli MG1655 ΔgalETKM Δagp P22 galK | 69, 08 |
| E. coli MG1655 ΔgalET P22 galK | 12, 48 |
| E. coli MG1655 ΔgalETKM Δagp | 64, 90 |
| P22 galK + orotate (2 g/L) | |

### Example 18. Production of Glucose-6-Phosphate Using Sucrose Phosphorylase in E. coli

By introducing sucrose phosphorylase sucrose is split into glucose-1-P and fructose. By additionally knocking-out genes coding for (a) phosphatase(s) (agp), glucose 6-phosphate-1-dehydrogenase (zwf), phosphoglucose isomerase (pgi), glucose-1-phosphate adenylyltransferase (glgC) a mutant is constructed which accumulates glucose-6-P.

The KO mutants are chosen in such a way that the metabolism is split into two disconnected parts. However, all possible combinations of these genes result in increased supply. Instead of knocking-out these genes, this goal is also achieved by rendering them defective or by reducing their expression.

By metabolically engineering E. coli a base strain is constructed that is an efficient producer of specialty carbohydrates and their derivatives whose pathway starts from Glucose-6-P (Table 13).

TABLE 13
Base strain glucose-6-phosphate using a sucrose phosphorylase

| Knock-out | Reaction |
|---|---|
| zwf | G6P + NADP → 6PG + NADPH |
| agp | G1P + H₂O → Glc + Pi |
| glgC | G1P + ATP + H ←> ADP-glucose + PPi |
| pgi | G6P ↔ F6P |
| Knock-in | Reaction |
| Sucrose | Suc + Pi → G1P + Fru |
| phosphorylase | |
| pgm | G6P ↔ G1P |

### Example 19. Production of Glucose-6-Phosphate Using Invertase in E. coli

By introducing sucrose hydrolase/invertase sucrose is split into glucose and fructose. By additionally knocking-out genes coding for (a) phosphatase(s) (agp), glucose 6-phosphate-1-dehydrogenase (zwf), phosphoglucose isomerase (pgi), glucose-1-phosphate adenylyltransferase (glgC), phosphoglucomutase (pgm) a mutant is constructed which accumulates Glucose-6-P (Table 14).

TABLE 14
Base strain glucose-6-phosphate using invertase in E. coli

| Knock-out | Reaction |
|---|---|
| zwf | G6P + NADP → 6PG + NADPH |
| agp | G1P + H₂O → Glu + Pi |
| pgi | G6P ↔ F6P |
| pgm | G6P ↔ G1P |
| Knock-in | Reaction |
| invertase | Suc + H₂O → Glc + Fru |

### Example 20. Production of Fructose-6-Phosphate Using Invertase in E. coli

By introducing sucrose hydrolase/invertase sucrose is split into glucose and fructose. By additionally knocking-out genes coding for (a) phosphatase(s) (agp), phosphofructokinase (pfkA and pfkB), phosphoglucose isomerase (pgi), glucose-1-phosphate adenylyltransferase (glgC), phosphoglucomutase (pgm) a mutant is constructed which accumulates fructose-6-phosphate (Table 15).

TABLE 15
Base strain fructose-6-phosphate

| Knock-out | Reaction |
|---|---|
| pfkA | F6P + ATP → FBP + ADP |
| pfkB | F6P + ATP → FBP + ADP |
| agp | G1P + H₂O → Glc + Pi |
| pgi | G6P ↔ F6P |
| pgm | G6P ↔ G1P |
| Knock-in | Reaction |
| invertase | Suc + H₂O → Glc + Fru |

### Example 21. Production of β-Glucose-1-Phosphate Using Maltose Phophorylase in E. coli

By introducing maltose phosphorylase, maltose is split into 3-D-glucose 1-phosphate and glucose. By additionally knocking-out genes coding for (a) phosphatase(s) (agp, yfbT), phosphoglucomutase (ycjU), maltose hydrolase (malPQ) a mutant is constructed which accumulates β-Glucose-1-P. The KO mutants are chosen in such a way that the metabolism is split into two disconnected parts. However, all possible combinations of these genes result in increased supply. Instead of knocking-out these genes, this goal is also achieved by rendering them defective or by reducing their expression (Table 16).

TABLE 16
Base strain β-D-Glucose-1-phosphate using maltose
phophorylase in E. coli

| Knock-out | Reaction |
|---|---|
| malPQ | Maltose → glucose |
| yfbT | β-D-glucose 1-phosphate + H₂O → |
| | glucose + Pi |
| agp | Glucose-1-phosphate + H₂O → |
| | glucose + Pi |
| ycjU | β-D-glucose 1-phosphate <=> |
| | β-D-glucose-6-phosphate |
| Knock-in | Reaction |
| Maltose | Maltose + Pi → Glucose-1P + Glucose |
| phosphorylase | |
| (MP) | |

### Example 22. Production of 3-Glucose-1-Phosphate Using Trehalose Phophorylase in E. coli

By introducing trehalose phosphorylase trehalose is split into β-D-glucose 1-phosphate and glucose. By additionally knocking-out genes coding for (a) phosphatase(s) (agp, yfbT), phosphoglucomutase (ycjU), undesired native trehalose degrading enzymes (treABC, treC, treE, treF) a mutant is constructed which accumulates β-Glucose-1-phosphate (Table 17).

The KO mutants are chosen in such a way that the metabolism is split into two disconnected parts. However, all possible combinations of these genes result in increased productivity. Instead of knocking-out these genes, this goal is achieved by rendering them defective or by reducing their expression.

TABLE 17
Base strain β-D-Glucose-1-phosphate using trehalose
phosphorylase in E. coli

| Knock-out | Reaction |
|---|---|
| treA | trehalose + H2O → 2 β-D-glucose |
| treC | trehalose 6-phosphate + H2O → β-D- |
| | glucose-6-phosphate + β-D-glucose |
| treE | trehalose 6-phosphate + H₂O ↔ β-D- |
| | glucose-6-phosphate + β-D-glucose |
| treF | trehalose + H2O ↔ β-D-glucose |
| yfbT | β-D-glucose 1-phosphate + H₂O → |
| | Glucose + Pi |
| agp | glucose 1-phosphate + H2O → Glucose + Pi |
| ycjU | β-D-glucose 1-phosphate ↔ β-D- |
| | glucose-6-phosphate |
| Knock-in | Reaction |
| Trehalose | Trehalose + Pi → β-D-Glucose-1P + Glucose |
| Phosphorylase | |
| (TP) | |
| otsB | Trehalose-6-phosphate + H₂O → |
| | trehalose + Pi |

### Example 23. Production of Kojibiose

By additionally introducing kojibiose phosphorylase in a strain accumulating β-D-glucose 1-phosphate and additionally knocking-out genes coding for glucokinase (glk) and, phosphotransferase system (ptsG) a mutant is constructed which produces kojibiose.

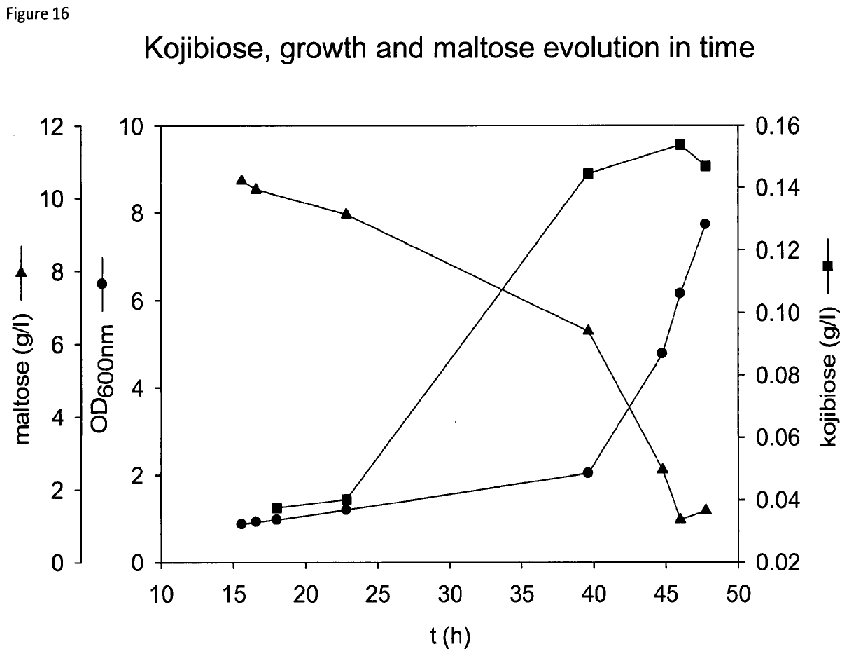

A fermentation is performed with an E. coli mutant strain (ΔlacZΔglgCΔagpΔptsGΔmalPQΔycjU pCXp22MPp22KP) using maltose and glucose as main carbon sources yielding kojibiose. (FIG. 16).

### Example 24. Production of UDP Glucose From Sucrose via a Sucrose Phosphorylase

By introducing sucrose phosphorylase (e.g., originating from Bifidobacterium adolescentis) sucrose is split into fructose and glucose-1-P. Starting from a glucose-1-phosphate accumulating strain (see examples above) and by additionally knocking-out genes coding UDP-glucose 4 epimerase (galE), UDP-glucose galactose-1-P uridilyltransferase (galT), 5′-nucleotidase/UDP-sugar hydrolase (ushA), UDP-glucose 6-dehydrogenase (ugd) a mutant is constructed which accumulates UDP-glucose by additionally overexpressing genes coding for UDP-glucose pyrophosphorylase (e.g. coming from Bifidobacterium bifidum) (Table 18).

TABLE 18
Base strain UDP-Glucose from sucrose via a sucrose
phosphorylase in E. coli

| Knock-out | Reaction |
|---|---|
| lacZ | Glu + Gal ↔ Lactose |
| galE | UDP-Glucose ↔ UDP-Galactose |
| galT | Gal1P + UDP-Glucose ↔ UDP- |
| | Galactose + G1P |
| ca | → colanic acid |
| ushA | a UDP-sugar + H₂O ↔ uridine-5′- |
| | phosphate + an α-D-aldose-1- |
| | phosphate + 2 H+ |
| ugd | 2 NAD+ + UDP-D-glucose + H₂O ↔ 2 |
| | NADH + UDP-D-glucuronate + 3 H+ |
| pgm | G1P ↔ G6P |
| glgC | G1P + ATP + H ←> ADP-glucose + PPi |
| agp | G1P + H₂O → Glu + Pi |
| ptsG | Glc + PEP → G6P + Pyr |
| glk | Glc + ATP → G6P + ADP |
| Knock-in | Reaction |
| galU/F | G1P + UTP-UDP-Glucose + PPi |
| Sucrose | Suc → G1P + Fru |
| phosphorylase | |

### Example 25. Production of UDP-Glucose in E. coli With a Sucrose-6-Phosphate Synthase Combined With Sucrose PTS

By introducing Sucrose PTS (94) and Sucrose-6-phosphate synthase (69), sucrose is converted into fructose-6-phosphate and UDP-glucose. Starting from the strain described in example4, without a sucrose phosphorylase, and by additionally knocking-out genes coding for (a) phosphatase(s) (agp), UDP-glucose 4 epimerase (galE), UDP-glucose galactose-1-P uridilyltransferase (galT), 5′-nucleotidase/UDP-sugar hydrolase (ushA), UDP-glucose 6-dehydrogenase (ugd) a mutant is constructed which accumulates UDP-glucose By additionally overexpressing genes coding for UDP-glucose pyrophosphorylase (Bifidobacterium bifidum) (Table 19).

TABLE 19
Base strain UDP-Glucose with sucrose-6-phosphate synthase
combined with sucrose PTS

| Knock-out | Reaction |
|---|---|
| galE | UDP-Glucose ↔ UDP-Galactose |
| galU/galF | UTP + G1P ↔ UDP-Glucose + PPi |
| galT | Gal1P + UDP-Glucose ↔ UDP- |
| | Galactose + G1P |
| ca | → colanic acid |
| ushA | a UDP-sugar + H₂O ↔ uridine-5′- |
| | phosphate + an α-D-aldose-1-phosphate + 2 H+ |
| ugd | 2 NAD+ + UDP-D-glucose + H₂O ↔ 2 |
| | NADH + UDP-D-glucuronate + 3 H+ |
| Knock-in | Reaction |
| Sucrose 6P | UDP-glucose + D-fructose 6-phosphate → |
| synthase | UDP + sucrose 6-phosphate |
| Sucrose PTS | Sucrose + PEP → sucrose-6-phosphate + Pyruvate |

### Example 26. Production of UDP Galactose via Galactokinase

By overexpressing genes coding for galactokinase (galk) and Galactose-1-phosphate uridylyitransferase (for example originating from Bifidobacterium bifidum) the formation of UDP-galactose is enhanced by additionally knocking-out genes coding for (a) phosphatase(s) (agp), UDP-glucose, galactose-1-P uridilyitransferase (galT), UDP-glucose-4-epimerase (galE) a mutant is constructed which accumulates galactose-1-P (Table 20).

TABLE 20
Base strain UDP-Galactose via galactokinase

| Knock-out | Reaction |
|---|---|
| galT | Gal1P + UDP-Glucose ↔ UDP- |
| | Galactose + G1P |
| galE | UDP-Glucose ↔ UDP-Galactose |
| galK | α-galactose + ATP → Gal1P + ADP |
| galM | β-galactose ↔ α-galactose |
| agp | glucose 1-phosphate + H2O → Pi + |
| | glucose |
| Knock-in | Reaction |
| galK | α-galactose + ATP → Gal1P + ADP |
| Galactose-1- | Gal1P + UTP → UDP-Galactose + PPi |
| phosphate | |
| uridylyltransferase | |

### Example 27. Production of UDP Galactose via Lactose Phosphorylase

By introducing lactose phosphorylase (20), lactose is split into glucose and galactose-1-P. By knocking-out genes coding for (a) phosphatase(s) (agp), UDP-glucose, galactose-1-P uridilyltransferase (galT), UDP-glucose-4-epimerase (galE) a mutant is constructed which accumulates galactose-1-P. By additionally overexpressing genes coding for Galactose-1-phosphate uridylyltransferase (for example coming from Bifidobacterium bifidum) the formation of UDP-galactose is enhanced (Table 21).

The KO mutants are chosen in such a way that the metabolism is split into two disconnected parts. However, all possible combinations of these genes result in increased productivity. Instead of knocking-out these genes, this goal is achieved by rendering them defective or by reducing their expression.

TABLE 21
Base strain UDP-Galactose via lactose phosphorylase

| Knock-out | Reaction |
|---|---|
| lacZ | Glu + Gal ↔ Lactose |
| galE | UDP-Glc <--> UDP-Gal |
| agp | G1P + H₂O → Glu + Pi |
| galT | UDP-Glc + Gal1P <--> UDP-Gal + G1P |
| Knock-in | Reaction |
| Lactose | Lactose + Pi → Gal1P + Glucose |
| phosphorylase | |
| Galactose-1- | Gal1P + UTP → UDP-Galactose + PPi |
| phosphate | |
| uridylyltransferase | |

### Example 28. Fructose-6-Phosphate Accumulation in E. coli

The metabolism is split in order to accumulate fructose-6-phosphate. This is achieved by knocking out the genes coding for phosphoglucose isomerase and phosphofructokinase activity. In E. coli these activities are coded by the genes pgi, pfkA and pfkB. The growth rate of strains devoid of these activities is described in Table 22 for growth on glucose and sucrose. The growth rate of the wild type strain is somewhat affected when grown on sucrose after introduction of a sucrose phosphorylase in comparison with growth on glucose, however the introduction of pgi knock outs and pfkA and pfkB double mutations lead to significant reduction of growth rate, with the latter being extremely low (0.02 h-1) on glucose. Surprisingly the mutant strain ΔpgiΔpfkAΔpfkB has a similar growth rate to that of the Δpgi single mutant.

TABLE 22
Specific growth rates of the glycolysis knock out strains on a
minimal medium with glucose andsucrose. In the strains grown
on sucrose a plasmid coding for sucrose phosphorylase was introduced.

| | Growth | Growth |
|---|---|---|
| | rate on | rate on |
| | glucose | sucrose |
| Strain | (h-1) | (h-1) |
| Wild type | 0.64 | 0.41 |
| Δpgi | 0.18 | 0.23 |
| ΔpfkAΔpfkB | 0.02 | n.d. |
| ΔpgiΔpfkAΔpfkB | 0.23 | 0.24 |

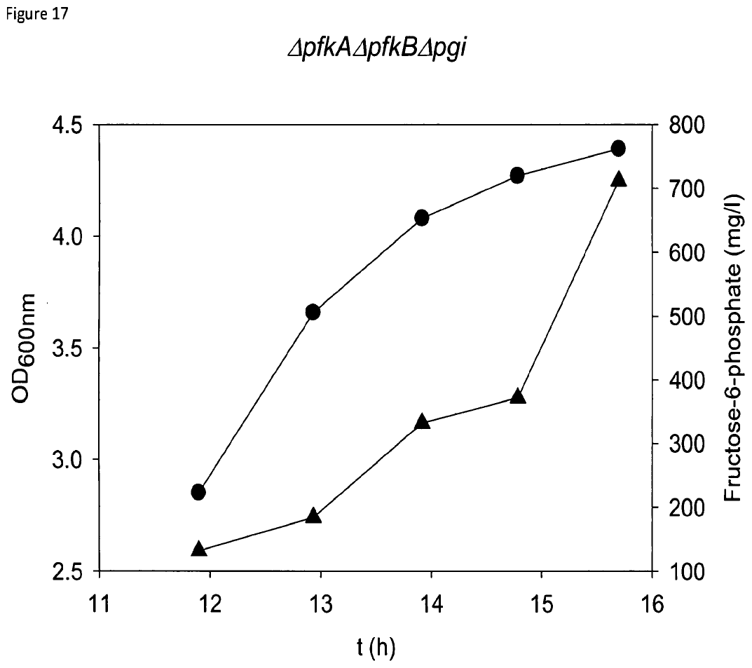

Only the ΔpgiΔpfkAΔpfkB mutant strain accumulated fructose-6-phosphate in the medium when grown on sucrose, the other strains did not indicate any F6P accumulation. The growth profile and F6P accumulation by this strain is shown FIG. 17.

### Example 29. αGlucose-1-Phosphate Accumulation in Saccharomyces cereviseae

Because Saccharomyces cereviseae splits sucrose by nature, all alternative sucrose degrading reactions (invertases), which are coded by the genes SUC2, MAL32, MAL12, YJL216C, YGR287c, are knocked out. To avoid the assimilation of α-glucose-1-phosphate, the enzymes that convert this activated carbohydrate are rendered less functional or non-functional. These enzymes are phosphoglucomutase, coded by PGM1 and PGM2, and glucose-1-phosphatase, coded by INM1 and INM2. By introducing a sucrose phosphorylase (e.g., originating from Bifidobacterium adolescentis), similar to the split metabolism of E. coli (see example above). The Saccharomyces cereviseae metabolism is split into two parts resulting in the accumulation of αGlucose-1-phosphate.

### Example 30. Cellobiose Production With Saccharomyces cereviseae

Because Saccharomyces cereviseae splits sucrose by nature, all alternative sucrose degrading reactions (invertases), which are coded by the genes SUC2, MAL32, MAL12, YJL216C, YGR287c, are knocked out. To avoid the assimilation of α-glucose-1-phosphate, the enzymes that convert this activated carbohydrate are rendered less functional or non-functional. These enzymes are phosphoglucomutase, coded by PGM1 and PGM2, and glucose-1-phosphatase, coded by INM1 and INM2. By introducing a sucrose phosphorylase (e.g., originating from Bifidobacterium adolescentis), similar to the split metabolism of E. coli (see example above). The Saccharomyces cereviseae metabolism is split into two parts resulting in the accumulation of αglucose-1-phosphate. By introducing a cellobiose phosphorylase gene originating from Cellulomonas uda, Saccharomyces cereviseae is able to produce cellobiose.

To avoid degradation of glucose, glucokinase activity, coded by GLK1 is knocked out as well. Because hexokinases in Saccharomyces cereviseae are not specific, these are replaced by a specific heterologous substrate specific hexokinase. Fructokinases originating from E. coli or Bifidobacterium adolescentis show lower activity for glucose and can replace the genes coding for the native hexokinases coded by HXK1 and HXK2.

### Example 31. Fructose-6-Phosphate Accumulation in Saccharomyces cereviseae by Introducing a Sucrose Phosphorylase

Because Saccharomyces cereviseae splits sucrose by nature, all alternative sucrose degrading reactions (invertases), which are coded by the genes SUC2, MAL32, MAL12, YJL216C, YGR287c, are knocked out. By introducing sucrose phophorylase from Bifidobacterium adolescentis sucrose is split into fructose and glucose-1-phosphate. To avoid the conversion of fructose-6-phosphate into biomass, the activity of the enzymes phosphoglucose isomerase and phosphofructokinase is reduced or eliminated by rendering the genes pgil, PFK1 and PFK2 less functional or non-functional, respectively.

### Example 32. Galactose-1-Phosphate Accumulation in Saccharomyces cereviseae

Galactose-1-phosphate is derived from the disaccharide lactose. Because Saccharomyces cereviseae does not split lactose by nature, a heterologous β-galactosidase (e.g. from E. coli) is introduced. This leads to the degradation of lactose to galactose and glucose. The goal is to increase the supply of galactose-1-phosphate to a biosynthetic pathway of a specialty carbohydrate. Therefore, galactose-1-phosphate may not be converted anymore into biomass, which is catalysed by UDP-glucose-hexose-1-phosphate uridylyltransferase, the aldose reductase. This is achieved by knocking out the genes coding for UDP-glucose-hexose-1-phosphate uridylyltransferase and aldose reductase, GAL7 and GRE3 respectively. To avoid degradation of said galactose-1-phosphate, the genes encoding for galactose-1-phosphatase are be knocked out. These are INM1 and INM2. A galactokinase is overexpressed in order to enhance the formation of galactose-1-phosphate from galactose.

### Example 33. Glucose-6-Phosphate Accumulation in Saccharomyces cereviseae via its Native Invertase

To split the Saccharomyces cereviseae metabolism into two parts (to enhance the supply of glucose-6-phosphate so that it can be used as a building block for a sppecialty carbohydrate) glucose-6-phosphate dehydrogenase, phosphoglucomutase and phosphoglucose isomerase, which are coded by the genes ZWF1, PGM1 and PGM2, and PGI1, respectively, are knocked out. In such a strain sucrose is split in fructose and glucose by the native invertases and phosphorylated into fructose-6-phosphate and glucose-6-phosphate by the native hexokinases. Glucose-6-phosphate is then supplied to the specialty carbohydrate biosynthesis pathway and fructose-6-phosphate is converted into biomass.

### Example 34. Glucose-6-Phosphate Accumulation in Saccharomyces cereviseae via Sucrose Phosphorylase

Saccharomyces cereviseae is modified to produce glucose-6-phosphate from sucrose with a sucrose phosphorylase originating from Bifidobacterium adolescentis. Because sucrose phosphorylase competes for sucrose with invertase, the genes coding for invertase activity are knocked out. These genes are SUC2, MAL32, MAL12, YJL216C, and YGR287c. Glucose-1-phosphate is then further converted into glucose-6-phosphate with a phosphoglucomutase coded by PGM1 and PGM2. To avoid degradation of glucose-1-phosphate into glucose, glucose-1-phosphatase encoding genes are knocked out, coded by INM1 and INM2. The assimilation of this activated saccharide is further reduced by eliminating the UTP glucose-1-phosphate uridylyltransferase activity in the cell, by rendering the genes YHL012W and UGP1less functional or non-functional. The fructose moiety is phosphorylated by the native hexokinases coded by HXK1 and HXK2 and converted into biomass.

### Example 35. Enhanced UDP-Glucose Formation in Saccharomyces cereviseae via Sucrose Synthase

Because Saccharomyces cereviseae splits sucrose by nature, all alternative sucrose degrading reactions (invertases), which are coded by the genes SUC2, MAL32, MAL12, YJL216C, YGR287c, are knocked out. A sucrose synthase, e.g. originating from Solanum tuberosum, is introduced so that sucrose is split into UDP-Glucose and fructose. To avoid UDP-glucose conversion into biomass, the activity of the enzymes UDP-glucose diphosphorylase, UDP-glucose 4-epimerase, UDP-glucose-hexose-1-phosphate uridylyltransferase, UDP-glucose-glycogen glucosyltransferase, UDP-glucose-1,3-beta-D-glucan glucosyltransferase, UDP-glucose-glucosephosphate glucosyltransferase are rendered less functional or non-functional, these enzymes are coded by the genes UGP1 and YHL012W, GAL10, GAL7, GSY1 and GSY2, FEN1 and FKS1 and GSC2, and TPS1, respectively. The fructose moiety is phosphorylated by the native hexokinases coded by HXK1 and HXK2 and converted into biomass.

### Example 36. Enhanced UDP-Glucose Formation in Saccharomyces cereviseae via Sucrose Sucrose Phosphorylase

In order to enhance UDP-glucose supply in Saccharomyces cereviseae, a strain as described in Example 29 is further modified by overexpressing the gene GAL7 which codes for a UTP-glucose-1-phosphate uridylyltransferase that catalyzes the conversion of α-glucose-1-phosphate into UDP-glucose.

### Example 37. Enhanced UDP-Galactose Formation in Saccharomyces cereviseae via 3-Galactosidase

In order to enhance UDP-galactose supply in Saccharomyces cereviseae, a strain as described in Example 32 is further modified by overexpressing a gene which codes for a UTP-galactose-1-phosphate uridylyltransferase (e.g. coming from Bifidobacterium bifidum) that catalyzes the conversion of a-galactose-1-phosphate into UDP-galactose.

### Example 38. Dictyostellium discoideum α1,2-fucosyltransferase Activity

### Introduction

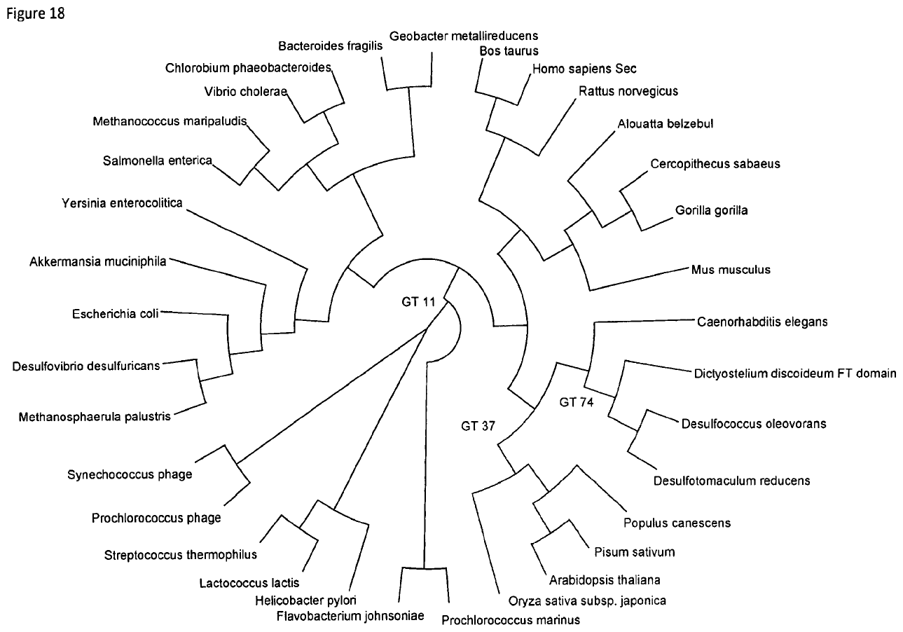

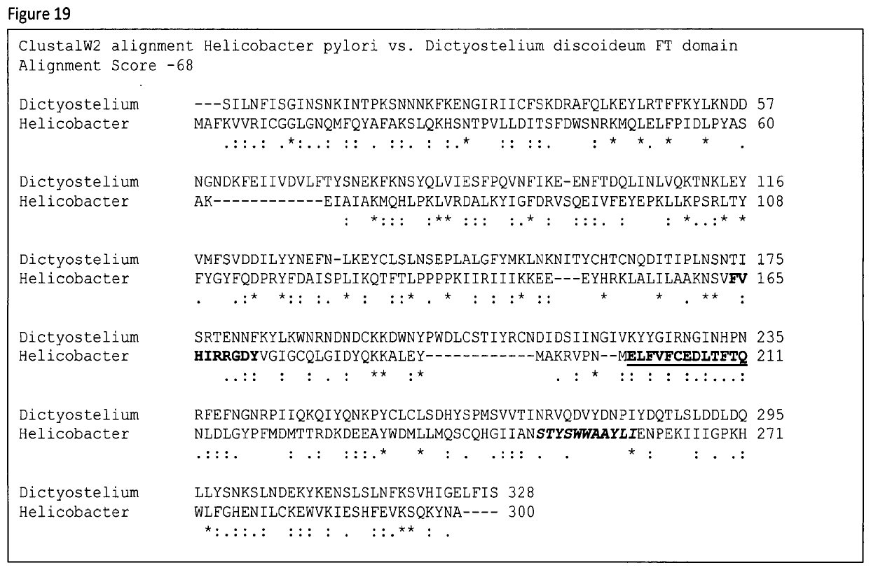

The Dictyostelium discoideum α1,2-fucosyltransferase (FT) is part of a rather new glycosyltransferase class. All known FT's belong to class GT11, while D. discoideum FT belongs to class GT74. Such an FT has, up to now, only been found in two other organisms namely Desulfococcus oleovorans and Desulfotomaculum reducens. The third class is GT37, which only contains plant FT's (FIG. 18). A clustalW and Tcoffee alignment indicated that the identity with H. pylori is only 9.7% and 15.5%, respectively. The conserved motives of GT11 are shown in FIG. 19 and these do not occur at all in the Dictyostelium protein (67). These domains differentiate for the fucosylation activity of the transferases. The first two motives are the same for α-1,2-fucosyltransferase and a-6-fucosyltransferase, while the third differentiates the enzymes from each other. α-1,3-fucosyltransferase contains two completely different conserved motives.

The Dictyostelium discoideum FT was however described to be only active on lacto-N-biose and not on galactose phenyl-β-galactoside, Galβ1-6-GlcNac, lactose, Galβ1-6Gal, Xyl, Glc, GlcNAc and GalNac (92).

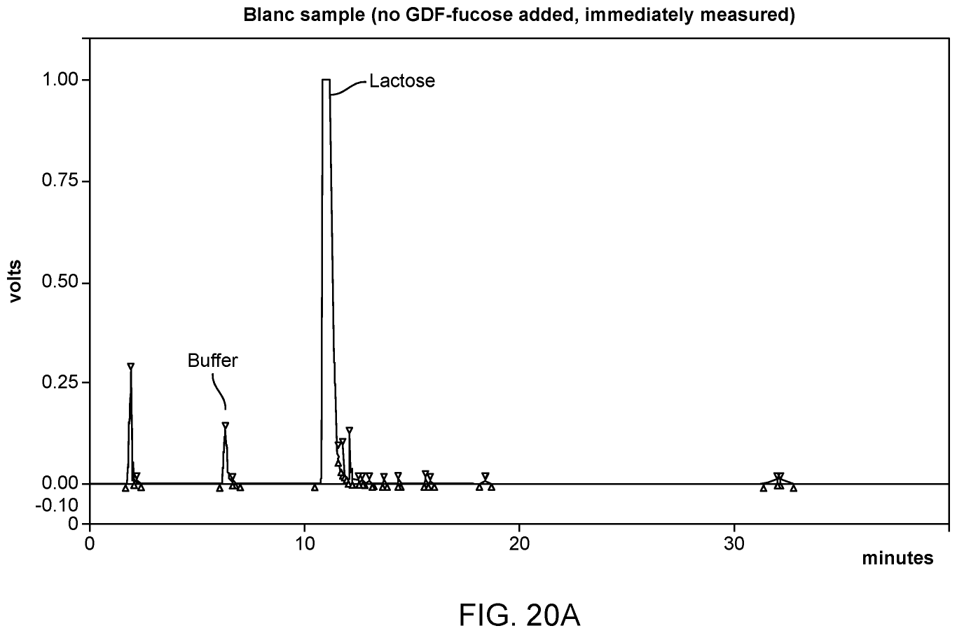

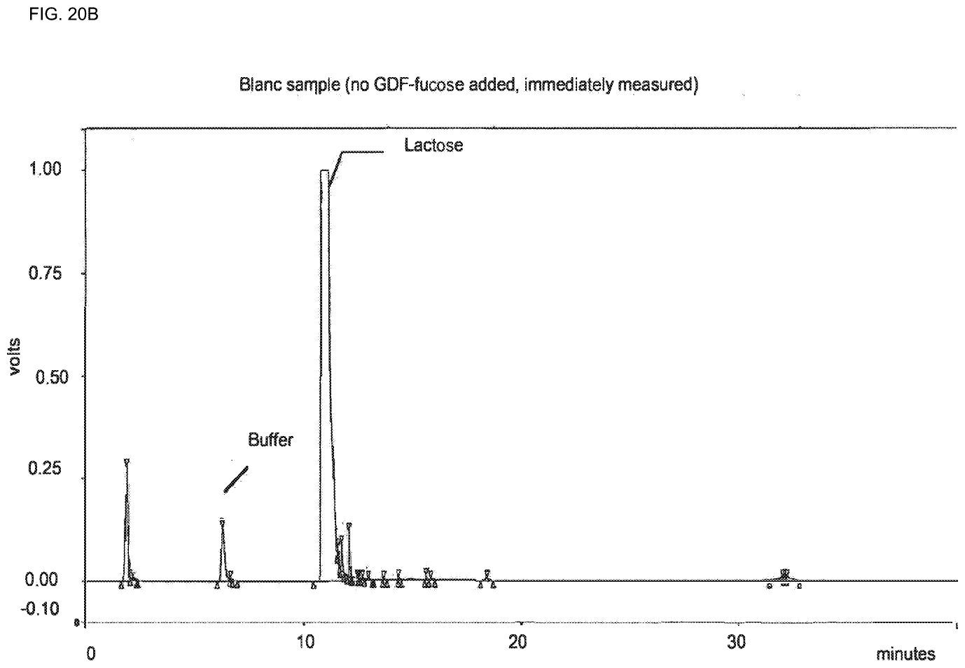

In FIGS. 20A and 20B a LC MSMS analysis of the Dictyostellium discoideum fucosyltransferase assay is shown. This assay showed surprisingly that this heterologous expressed fucosyltransferase is active with lactose as acceptor and GDP-fucose as fucose donor. The activity of this enzyme was 0.19±0.01 U/mg protein and was determined following the method of Persson and Palcic (68).

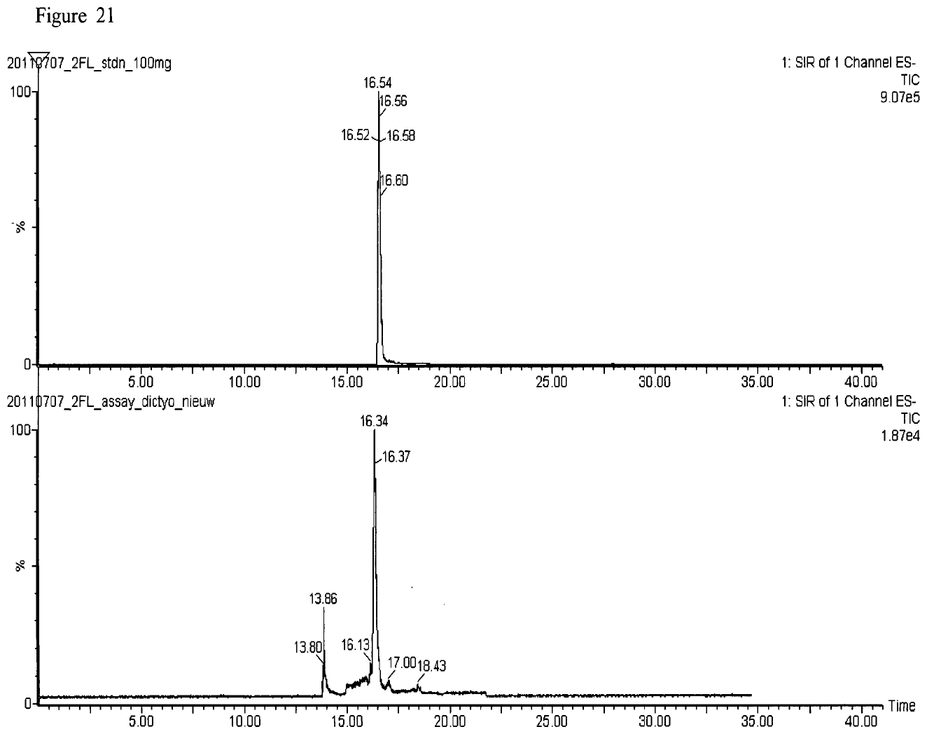

Because only half of the enzyme of Dictyostelium discoideum is responsible for its α1,2-fucosyltransferase activity, this part was cloned in a similar way to the complete enzyme, however with an additional start codon (coded by ATG) in front of the nucleotide sequence. This new enzyme was produced in a ΔlacZΔglgCΔmanAΔCA mutant strain and assayed for α1,2-fucosyltransferase activity with an LC MSMS. FIG. 21 shows clearly that this partial enzyme is still able to form 2-fucosyllactose from GDP-fucose and lactose.

### Example 39. Myo-Inositol Production in E. coli

Starting from a base strain that accumulates glucose-6-phosphate, a myo-inositol producer is constructed by additionally introducing a gene coding for myo-inositol-1-phosphate synthase (INO1, originating from Saccharomyces cerevisiae) and myo-inositol-1 (or 4)-monophosphatase (INM1 and INM2, originating from Saccharomyces cerevisiae).

Example 40. Lacto-N-biose production in E. coli

Starting from a base strain that accumulates galactose-1-phosphate, a Lacto-N-biose producer is constructed by additionally introducing a gene coding for lacto-N-biose phosphorylase (Inbp, Bifidobacterium longum). A fermentation is performed using lactose and N-acetylglucoamine as carbon sources. Degradation of N-acetylglucosamine is inhibited in the produer strain by eliminating any N-acetyl-D-glucosamine kinase activity (nagk) and N-acetylglucosamine PTS activity (nagE, ptsH, ptsl, manXYZ).

### LIST OF ABBREVIATIONS USED IN THE TEXT

3KO a mutant strain in which the genes pgm, lacZ and glgC are knocked out
4KO a mutant strain in which the genes pgm, lacZ, glgC and agp are knocked out
6PG 6-phosphogluconate
BA Bifidobacterium adolescentis
BaSP a sucrose phosphorylase originating from Bifidobacterium adolescentis
CA Colanic acid operon defined by Stevenson et al. (86)
CDW Cell dry weight
CuCP Cellulomonas uda cellobiose phosphorylase
F6P Fructose-6-phosphate
FBP Fructose-1,6-bisphosphate
Fru Fructose
FT a1,2-Fucosyltransferase
G1P Glucose-1-phosphate
Gal1P Galactose-1-phosphate
Glc Glucose
Glu Glucose
KI Knock in
KO Knock out
KP Kojibiose phosphorylase
LA Lactobacillus acidophilus
LB Luria Bertani Broth
LM Leuconostoc mesenteroides
MP Maltose phosphorylase
P22 promoter 22 of the promoter library of De Mey et al (2007)
pCXp22 a plasmide that contains the P22 promoter according to Aerts et al (1)
PEP Phosphoenolpyruvate
Pi Inorganic phosphate
PPi Pyrophosphate
Pyr Pyruvate
rpm rotations per minute
SM Streptococcus mutans
SP Sucrose phosphorylase
Suc Sucrose
UDP-gal UDP-galactose
UDP-glc UDP-glucose
WT Wild type strain

### REFERENCES

1. Aerts, D., T. Verhaeghe, M. De Mey, T. Desmet, and W. Soetaert. 2010. A constitutive expression system for high throughput screening. Engineering in Life Sciences 10:DOI: 10.1002/elsc.201000065.
2. Agrawal, N., P. V. N. Dasaradhi, A. Mohmmed, P. Malhotra, R. K. Bhatnagar, and S. K. Mukherjee. 2003. RNA Interference: Biology, Mechanism, and Applications. Microbiology and Molecular Biology Reviews 67:657-685.
3. Antoine, T., C. Bosso, A. Heyraud, and E. Samain. 2005. Large scale in vivo synthesis of globotriose and globotetraose by high cell density culture of metabolically engineered Escherichia coli. Biochimie 87:197-203.
4. Avihoo, A., I. Gabdank, M. Shapira, and D. Barash. 2007. In silico design of small RNA switches. IEEE Transactions on Nanobioscience 6:4-11.
5. Ayres, E. K., V. J. Thomson, G. Merino, D. Balderes, and D. H. Figurski. 1993. Precise deletions in large bacterial genomes by Vector-mediated Excision (VEX): The trfA gene of promiscuous plasmid RK2 is essential for replication in several gram-negative hosts. Journal of Molecular Biology 230:174-185.
6. Badet, B., P. Vermoote, P. Y. Haumont, F. Lederer, and F. Le Goffic. 1987. Glucosamine synthetase from Escherichia coli: purification, properties, and glutamine-utilizing site location. Biochemistry 26:1940-1948.
7. Balbás, P., M. Alexeyev, I. Shokolenko, F. Bolivar, and F. Valle. 1996. A pBRINT family of plasmids for integration of cloned DNA into the Escherichia coli chromosome. Gene 172:65-69.
8. Balbas, P., and G. Gosset. 2001. Chromosomal editing in Escherichia coli. Molecular Biotechnology 19:1-12.
9. Beauprez, J. 2010. Metabolic modelling and engineering of Escherichia coli for succinate production. PhD. Ghent University, Ghent.
10. Boles, E., W. Liebetrau, M. Hofmann, and F. K. Zimmermann. 1994. A family of hexosephosphate mutases in Saccharomyces cerevisiae. European Journal of Biochemistry 220:83-96.
11. Buchholz, A., R. Takors, and C. Wandrey. 2001. Quantification of Intracellular Metabolites in Escherichia coli K12 Using Liquid Chromatographic-Electrospray Ionization Tandem Mass Spectrometric Techniques. Analytical Biochemistry 295:129-137.
12. Burda, P., and M. Aebi. 1998. The ALG10 locus of Saccharomyces cerevisiae encodes the \hat{l}±−1,2 glucosyltransferase of the endoplasmic reticulum: the terminal glucose of the lipid-linked oligosaccharide is required for efficient N-linked glycosylation. Glycobiology 8:455-462.
13. Byun, S.-G., M.-D. Kim, W.-H. Lee, K.-J. Lee, N. Han, and J.-H. Seo. 2007. Production of GDP-l-fucose, l-fucose donor for fucosyloligosaccharide synthesis, in recombinant Escherichia coli. Applied Microbiology and Biotechnology 74:768-775.
14. Cherepanov, P. P., and W. Wackernagel. 1995. Gene disruption in Escherichia coli: TcR and KmR cassettes with the option of Flp-catalyzed excision of the antibiotic-resistance determinant. Gene 158:9-14.
15. Chow, T., M. J. Goldenthal, J. D. Cohen, M. Hegde, and J. Marmur. 1983. Identification and physical characterization of yeast maltase structural genes. Molecular and General Genetics MGG 191:366-371.
16. Chow, T. H. C., P. Sollitti, and J. Marmur. 1989. Structure of the multigene family of &lt;i&gt;MAL&lt;/i&gt; loci in &lt;i&gt;Saccharomyces&lt;/i&gt. Molecular and General Genetics MGG 217:60-69.
17. Czar, M. J., J. C. Anderson, J. S. Bader, and J. Peccoud. 2009. Gene synthesis demystified. Trends in Biotechnology 27:63-72.
18. Daran, J. M., N. Dallies, D. Thines-Sempoux, V. Paquet, and J. François. 1995. Genetic and Biochemical Characterization of the UGP1 Gene Encoding the UDP-Glucose Pyrophosphorylase from Saccharomyces cerevisiae. European Journal of Biochemistry 233:520-530.
19. Datsenko, K. A., and B. L. Wanner. 2000. One-step inactivation of chromosomal genes in Escherichia coli K-12 using PCR products. Proceedings of the national academy of sciences of the United States of America 97:6640-6645.
20. De Groeve, M., V. Depreitere, T. Desmet, and W. Soetaert. 2009. Enzymatic production of a-D-galactose 1-phosphate by lactose phosphorolysis. Biotechnology Letters 31:1873-1877.
21. De Mey, M. 2007. Metabolic modelling and engineering of Escherichia coli to minimize acetate formation in recombinant DNA fermentation processes. Ghent University, Ghent.
22. De Mey, M., A. Cerdobbel, K. Van Nieuland, J. Maertens, W. Soetaert, and E. J. Vandamme. 2006. Promoter Engineering: A Useful Tool for Fine Tunning Gene Expression in Escherichia coli. Department of Biochemical and Microbial Technology, Ghent University.
23 De Mey, M., J. Maertens, G. J. Lequeux, W. K. Soetaert, and E. J. Vandamme. 2007. Construction and model-based analysis of a promoter library for E. coli: an indispensable tool for metabolic engineering. BMC Biotechnology 7:34-48.
24. De Virgilio, C., N. BÜRckert, W. Bell, P. JenÖ, T. Boller, and A. Wiemken. 1993. Disruption of TPS2, the gene encoding the 100-kDa subunit of the trehalose-6-phosphate synthase/phosphatase complex in Saccharomyces cerevisiae, causes accumulation of trehalose-6-phosphate and loss of trehalose-6-phosphate phosphatase activity. European Journal of Biochemistry 212:315-323.
25. Dedhia, N. N., T. Hottiger, and J. E. Bailey. 1994. Overproduction of glycogen in Escherichia coli blocked in the acetate pathway improves cell growth. Biotechnology and Bioengineering 44:132-139.
26. Dickinson, J. R. 1991. Biochemical and genetic studies on the function of, and relationship between, the PGI1-and CDC30-encoded phosphoglucose isomerases in Saccharomyces cerevisiae. Journal of General Microbiology 137:765-770.
27. Dippel, R., and W. Boos. 2005. The Maltodextrin System of Escherichia coli: Metabolism and Transport. J. Bacteriol. 187:8322-8331.
28. Edwards, C. J., D. J. Innes, D. M. Burns, and I. R. Beacham. 1993. UDP-sugar hydrolase enzymes in Salmonella enterica and Escherichia coli: silent alleles of ushA in related strains of group I Salmonella isolates, and of ushB in wild type and K12 strains of Escherichia coli indicate recent and early silencing events, respectively I. Fems Microbiology Letters 114:293-298.
29. Egan, S. E., R. Fliege, S. Tong, A. Shibata, R. E. Wolf, Jr., and T. Conway. 1992. Molecular characterization of the Entner-Doudoroff pathway in Escherichia coli: sequence analysis and localization of promoters for the edd-eda operon. Journal of Bacteriology 174:4638-4646.
30. Farkas, I., T. A. Hardy, A. A. DePaoli-Roach, and P. J. Roach. 1990. Isolation of the GSY1 gene encoding yeast glycogen synthase and evidence for the existence of a second gene. Journal of Biological Chemistry 265:20879-20886.
31. Farkas, I., T. A. Hardy, M. G. Goebl, and P. J. Roach. 1991. Two glycogen synthase isoforms in Saccharomyces cerevisiae are coded by distinct genes that are differentially controlled. Journal of Biological Chemistry 266:15602-15607.
32. Flores, N., L. Leal, J. C. Sigala, R. de Anda, A. Escalante, A. Martinez, O. T. Ramirez, G. Gosset, and F. Bolivar. 2007. Growth recovery on glucose under aerobic conditions of an Escherichia coli strain carrying a phosphoenolpyruvate: Carbohydrate phosphotransferase system deletion by inactivating arcA and overexpressing the genes coding for glucokinase and galactose permease. Journal of Molecular Microbiology and Biotechnology 13:105-116.
33. Goedl, C., A. Schwarz, A. Minani, and B. Nidetzky. 2007. Recombinant sucrose phosphorylase from Leuconostoc mesenteroides: Characterization, kinetic studies of transglucosylation, and application of immobilised enzyme for production of a-D-glucose 1-phosphate. Journal of Biotechnology 129:77-86.
34. Gorsich, S., B. Dien, N. Nichols, P. Slininger, Z. Liu, and C. Skory. 2006. Tolerance to furfural-induced stress is associated with pentose phosphate pathway genes ZWF1, GND1, RPE1, and TKL1 in Saccharomyces cerevisiae. Applied Microbiology and Biotechnology 71:339-349.
35. Gräslund, S., P. Nordlund, J. Weigelt, B. M. Hallberg, J. Bray, O. Gileadi, S. Knapp, U. Oppermann, C. Arrowsmith, R. Hui, J. Ming, S. dhe-Paganon, H.-w. Park, S. Alexei, A. Yee, A. Edwards, R. Vincentelli, C. Cambillau, R. Kim, S.-H. Kim, Z. Rao, Y. Shi, T. C. Terwilliger, C.-Y. Kim, L.-W. Hung, G. S. Waldo, Y. Peleg, S. Albeck, T. Unger, O. Dym, J. Prilusky, J. L. Sussman, R. C. Stevens, S. A. Lesley, I. A. Wilson, A. Joachimiak, F. Collart, I. Dementieva, M. I. Donnelly, W. H. Eschenfeldt, Y. Kim, L. Stols, R. Wu, M. Zhou, S. K. Burley, J. S. Emtage, J. M. Sauder, D. Thompson, K. Bain, J. Luz, T. Gheyi, F. Zhang, S. Atwell, S. C. Almo, J. B. Bonanno, A. Fiser, S. Swaminathan, F. W. Studier, M. R. Chance, A. Sali, T. B. Acton, R. Xiao, L. Zhao, L. C. Ma, J. F. Hunt, L. Tong, K. Cunningham, M. Inouye, S. Anderson, H. Janjua, R. Shastry, C. K. Ho, D. Wang, H. Wang, M. Jiang, G. T. Montelione, D. I. Stuart, R. J. Owens, S. Daenke, A. Schutz, U. Heinemann, S. Yokoyama, K. Büssow, and K. C. Gunsalus. 2008. Protein production and purification. Nature Methods 5:135-146.
36. Hashimoto, H., A. Sakakibara, M. Yamasaki, and K. Yoda. 1997. Saccharomyces cerevisiae VIG9 Encodes GDP-mannose Pyrophosphorylase, Which Is Essential for Protein Glycosylation. Journal of Biological Chemistry 272:16308-16314.
37. Hebert, C. G., J. J. Valdes, and W. E. Bentley. 2008. Beyond silencing—engineering applications of RNA interference and antisense technology for altering cellular phenotype. Current opinion in biotechnology 19:500-505.
38. Heillisch, J., R. G. Ritzel, R. C. von Borstel, A. Aguilera, R. Rodicio, and F. K. Zimmermann. 1989. The phosphofructokinase genes of yeast evolved from two duplication events. Gene 78:309-321.
39. Heinisch, J. 1986. Isolation and characterization of the two structural genes coding for phosphofructokinase in yeast. Molecular and General Genetics MGG 202:75-82.
40. Hoang, T. T., R. R. Karkhoff-Schweizer, A. J. Kutchma, and H. P. Schweizer. 1998. A broad-host-range Flp-FRT recombination system for site-specific excision of chromosomally-located DNA sequences: application for isolation of unmarked Pseudomonas aeruginosa mutants. Gene 212:77-86.
41. Ishihara, S., A. Hirata, S. Nogami, A. Beauvais, J.-P. Latge, and Y. Ohya. 2007. Homologous Subunits of 1,3-Beta-Glucan Synthase Are Important for Spore Wall Assembly in Saccharomyces cerevisiae. Eukaryotic Cell 6:143-156.
42. Keasling, J. D. 1999. Gene-expression tools for the metabolic engineering of bacteria. Trends in Biotechnology 17:452-460.
43. Kiino, D. R., R. Licudine, K. Wilt, D. H. Yang, and L. B. Rothman-Denes. 1993. A cytoplasmic protein, NfrC, is required for bacteriophage N4 adsorption. Journal of Bacteriology 175:7074-7080.
44. Kim, C., S. Song, and C. Park. 1997. The D-allose operon of Escherichia coli K-12. J. Bacteriol. 179:7631-7637.
45. Kogure, T., N. Wakisaka, H. Takaku, and M. Takagi. 2007. Efficient production of 2-deoxy-scyllo-inosose from D-glucose by metabolically engineered recombinant Escherichia coli. Journal of Biotechnology 129:502-509.
46. Kornberg, H. L. 2001. Routes for fructose utilization by Escherichia coli. Journal of Molecular Microbiology and Biotechnology 3:355-359.
47. Kristensen, C. S., L. Eberl, J. M. Sanchez-Romero, M. Givskov, S. Molin, and V. De Lorenzo. 1995. Site-specific deletions of chromosomally located DNA segments with the multimer resolution system of broad-host-range plasmid RP4. Journal of Bacteriology 177:52-58.
48. Kuznetsova, E., M. Proudfoot, C. F. Gonzalez, G. Brown, M. V. Omelchenko, I. Borozan, L. Carmel, Y. I. Wolf, H. Mori, A. V. Savchenko, C. H. Arrowsmith, E. V. Koonin, A. M. Edwards, and A. F. Yakunin. 2006. Genome-wide Analysis of Substrate Specificities of the Escherichia coli Haloacid Dehalogenase-like Phosphatase Family. Journal of Biological Chemistry 281:36149-36161.
49. Lasserre, J.-P., E. Beyne, S. Pyndiah, D. Lapaillerie, S. Claverol, and M. Bonneu. 2006. A complexomic study of Escherichia coli using two-dimensional blue native/SDS polyacrylamide gel electrophoresis. Electrophoresis 27:3306-3321.
50. Llull, D., E. Garcia, and R. Lopez. 2001. Tts, a Processive b-Glucosyltransferase of Streptococcus pneumoniae, Directs the Synthesis of the Branched Type 37 Capsular Polysaccharide in Pneumococcus and Other Gram-positive Species. Journal of Biological Chemistry 276:21053-21061.
51. Lopez, F., M. Leube, R. Gil-Mascarell, J. P. Navarro-Aviñó, and R. Serrano. 1999. The yeast inositol monophosphatase is a lithium-and sodium-sensitive enzyme encoded by a non-essential gene pair. Molecular Microbiology 31:1255-1264.
52. Maitra, U. S., and H. Ankel. 1973. The intermediate in the uridine diphosphate galactose 4-epimerase reaction: Resolution of an apparent ambiguity. Journal of Biological Chemistry 248:1477-1479.
53. Markovitz, A., R. J. Sydiskis, and M. M. Lieberman. 1967. Genetic and biochemical studies on mannose-negative mutants that are deficient in phosphomannose isomerase in Escherichia coli K-12. Journal of Bacteriology 94:1492-1496.
54. Marolda, C., L., and M. Valvano, A. 1996. The GaIF protein of Escherichia coli is not a UDP-glucose pyrophosphorylase but interacts with the GalU protein possibly to regulate cellular levels of UDP-glucose. Molecular Microbiology 22:827-840.
55. Marquardt, J. L., E. D. Brown, C. T. Walsh, and K. S. Anderson. 1993. Isolation and structural elucidation of a tetrahedral intermediate in the UDP-N-acetylglucosamine enolpyruvoyl transferase enzymic pathway. Journal of the American Chemical Society 115:10398-10399.
56. Mazur, P., N. Morin, W. Baginsky, M. el-Sherbeini, J. Clemas, J. Nielsen, and F. Foor. 1995. Differential expression and function of two homologous subunits of yeast 1,3-beta-D-glucan synthase. Mol. Cell. Biol. 15:5671-5681.
57. Meijer, W. H., I. J. van der Klei, M. Veenhuis, and J. A. K. W. Kiel. 2007. ATG Genes Involved in Non-Selective Autophagy are Conserved from Yeast to Man, but the Selective Cvt and Pexophagy Pathways also Require Organism-Specific Genes. Autophagy 3:106-116.
58. Moretti, S., F. Armougom, I. M. Wallace, D. G. Higgins, C. V. Jongeneel, and C. Notredame. 2007. The M-Coffee web server: a meta-method for computing multiple sequence alignments by combining alternative alignment methods. Nucleic Acids Research 35: W645-W648.
59. Mu, J., C. Cheng, and P. J. Roach. 1996. Initiation of Glycogen Synthesis in Yeast. Journal of Biological Chemistry 271:26554-26560.
60. Müller, S., F. K. Zimmermann, and E. Boles. 1997. Mutant studies of phosphofructo-2-kinases do not reveal an essential role of fructose-2, 6-bisphosphate in the regulation of carbon fluxes in yeast cells. Microbiology 143:3055-3061.
61. Murray, M., and M. L. Greenberg. 2000. Expression of yeast INM1 encoding inositol monophosphatase is regulated by inositol, carbon source and growth stage and is decreased by lithium and valproate. Molecular Microbiology 36:651-661.
62. Nassau, P. M., S. L. Martin, R. E. Brown, A. Weston, D. Monsey, M. R. McNeil, and K. Duncan. 1996. Galactofuranose biosynthesis in Escherichia coli K-12: identification and cloning of UDP-galactopyranose mutase. Journal Of Bacteriology 178:1047-1052.
63. Neidhardt, F. C. 1996. Escherichia coli and Salmonella. Cellular and Molecular Biology. ASM Press, Washington, D.C.
64. Nogae, I., and M. Johnston. 1990. Isolation and characterization of the ZWF1 gene of Saccharomyces cerevisiae, encoding glucose-6-phosphate dehydrogenase. Gene 96:161-169.
65. Novotny, M. J., J. Reizer, F. Esch, and M. H. Saier, Jr. 1984. Purification and properties of D-mannitol-1-phosphate dehydrogenase and D-glucitol-6-phosphate dehydrogenase from Escherichia coli. J. Bacteriol. 159:986-990.
66. Oh, C.-S., D. A. Toke, S. Mandala, and C. E. Martin. 1997. ELO2 and ELO3, Homologues of theSaccharomyces cerevisiae ELO1 Gene, Function in Fatty Acid Elongation and Are Required for Sphingolipid Formation. Journal of Biological Chemistry 272:17376-17384.
67. Oriol, R., R. Mollicone, A. Cailleau, L. Balanzino, and C. Breton. 1999. Divergent evolution of fucosyltransferase genes from vertebrates, invertebrates, and bacteria. Glycobiology 9:323-334.
68. Persson, M., and M. M. Palcic. 2008. A high-throughput pH indicator assay for screening glycosyltransferase saturation mutagenesis libraries. Analytical Biochemistry 378:1-7.
69. Porchia, A. C., and G. L. Salerno. 1996. Sucrose biosynthesis in a prokaryotic organism: Presence of two sucrose-phosphate synthases in Anabaena with remarkable differences compared with the plant-enzymes. Proceedings of the National Academy of Sciences 93:13600-13604.
70. Priem, B., M. Gilbert, W. W. Wakarchuk, A. Heyraud, and E. Samain. 2002. A new fermentation process allows large-scale production of human milk oligosaccharides by metabolically engineered bacteria. Glycobiology 12:235-240.
71. Ramakrishnan, B., and P. K. Qasba. 2001. Crystal structure of lactose synthase reveals a large conformational change in its catalytic component, the [beta]1,4-galactosyltransferase-l. Journal of Molecular Biology 310:205-218.
72. Ramakrishnan, B., P. S. Shah, and P. K. Qasba. 2001. a-Lactalbumin (LA) Stimulates Milk b-1,4-Galactosyltransferase l (b4Gal-T1) to Transfer Glucose from UDP-glucose to N-Acetylglucosamine. Journal of Biological Chemistry 276:37665-37671.
73. Rasmussen, L., H. Sperling-Petersen, and K. Mortensen. 2007. Hitting bacteria at the heart of the central dogma: sequence-specific inhibition. Microbial Cell Factories 6:24.
74. Rider, M. H., L. Bertrand, D. Vertommen, P. A. Michels, G. G. Rousseau, and L. Hue. 2004. 6-phosphofructo-2-kinase/fructose-2,6-bisphosphatase: Head-to-head with a bifunctional enzyme that controls glycolysis. Biochemical Journal 381:561-579.
75. Riitta, V., and V. Martti. 1995. Comparison of the phenotypes of the IpxA and IpxD mutants of Escherichia coli. FEMS Microbiology Letters 134:227-232.
76. Rodriguez, A., T. De La Cera, P. Herrero, and F. Moreno. 2001. The hexokinase 2 protein regulates the expression of the GLK1, HXK1 and HXK2 genes of Saccharomyces cerevisiae. Biochem. J. 355:625-631.
77. Ruffing, A., and R. R. Chen. 2006. Metabolic engineering of microbes for oligosaccharide and polysaccharide synthesis. Microbial Cell Factories 5.
78. Rush, J. S., P. D. Rick, and C. J. Waechter. 1997. Polyisoprenyl phosphate specificity of UDP-GlcNAc: undecaprenyl phosphate N-acetylglucosaminyl 1-P transferase from E.coli. Glycobiology 7:315-322.
79. Sauer, B. 1987. Functional expression of the cre-lox site-specific recombination system in the yeast Saccharomyces cerevisiae. Molecular and Cellular Biology 7:2087-2096.
80. Schweizer, H. P. 2003. Applications of the Saccharomyces cerevisiae Flp-FRT system in bacterial genetics. Journal of Molecular Microbiology and Biotechnology 5:67-77.
81. Segawa, T., and T. Fukasawa. 1979. The enzymes of the galactose cluster in Saccharomyces cerevisiae. Purification and characterization of galactose-1-phosphate uridylyltransferase. Journal of Biological Chemistry 254:10707-10709.
82. Sinnott, M. L. 1978. lons, ion-pairs and catalysis by the lacZ b-galactosidase of Escherichia coli. FEBS Letters 94:1-9.
83. Smith, D. J., A. Proudfoot, L. Friedli, L. S. Klig, G. Paravicini, and M. A. Payton. 1992. PMI40, an intron-containing gene required for early steps in yeast mannosylation. Mol. Cell. Biol. 12:2924-2930.
84. Stagljar, I., S. te Heesen, and M. Aebi. 1994. New phenotype of mutations deficient in glucosylation of the lipid-linked oligosaccharide: cloning of the ALG8 locus. Proceedings of the National Academy of Sciences 91:5977-5981.
85. Sternberg, N., D. Hamilton, and R. Hoess. 1981. Bacteriophage P1 site-specific recombination: II. Recombination between loxP and the bacterial chromosome. Journal of Molecular Biology 150:487-507.
86. Stevenson, G., K. Andrianopoulos, M. Hobbs, and P. R. Reeves. 1996. Organization of the Escherichia coli K-12 gene cluster responsible for production of the extracellular polysaccharide colanic acid. Journal of Bacteriology 178:4885-4893.
87. Stevenson, G., B. Neal, D. Liu, M. Hobbs, N. H. Packer, M. Batley, J. W. Redmond, L. Lindquist, and P. Reeves. 1994. Structure of the O antigen of Escherichia coli K-12 and the sequence of its rfb gene cluster. Journal of Bacteriology 176:4144-4156.
88. Tallon, R., P. Bressollier, and M. C. Urdaci. 2003. Isolation and characterization of two exopolysaccharides produced by Lactobacillus plantarum EP56. Research in Microbiology 154:705-712.
89. Taussig, R., and M. Carlson. 1983. Nucleotide sequence of The yeast SUC2 gene for invertase. Nucleic Acids Research 11:1943-1954.
90. Teste, M.-A., J. M. François, and J.-L. Parrou. 2010. Characterization of a New Multigene Family Encoding Isomaltases in the Yeast Saccharomyces cerevisiae, the IMA Family. Journal of Biological Chemistry 285:26815-26824.
91. Thoden, J. B., and H. M. Holden. 2005. The Molecular Architecture of Galactose Mutarotase/UDP-Galactose 4-Epimerase from Saccharomyces cerevisiae. Journal of Biological Chemistry 280:21900-21907.
92. Trinchera, M., and S. Bozzaro. 1996. Dictyostelium cytosolic fucosyltransferase synthesizes H type 1 trisaccharide in vitro. FEBS Letters 395:68-72.
93. Tsuda, M. 1998. Use of a transposon-encoded site-specific resolution system for construction of large and defined deletion mutations in bacterial chromosome. Gene 207:33-41.
94. Wang, J., E. D. Gilles, J. W. Lengeler, and K. Jahreis. 2001. Modeling of inducer exclusion and catabolite repression based on a PTS-dependent sucrose and non-PTS-dependent glycerol transport systems in Escherichia coli K-12 and its experimental verification. Journal of Biotechnology 92:133-158.
95. Watzele, G., and W. Tanner. 1989. Cloning of the glutamine: fructose-6-phosphate amidotransferase gene from yeast. Pheromonal regulation of its transcription. Journal of Biological Chemistry 264:8753-8758.
96. Wedekind, J. E., P. A. Frey, and I. Rayment. 1996. The Structure of nucleotidylated histidine-166 of galactose-1-phosphate uridylyltransferase provides insight into phosphoryl group Transfer. Biochemistry 35:11560-11569.
97. Weissborn, A. C., Q. Liu, M. K. Rumley, and E. P. Kennedy. 1994. UTP: alpha-D-glucose-1-phosphate uridylyltransferase of Escherichia coli: isolation and DNA sequence of the galU gene and purification of the enzyme. Journal of Bacteriology 176:2611-2618.
98. Williams, J., J. Luke, and C. Hodgson. 2009. Strain engineering by genome mass transfer: Efficient chromosomal trait transfer method utilizing donor genomic DNA and recipient recombineering hosts. Molecular Biotechnology 43:41-51.
99. Yamamoto, K., A. Nakayama, Y. Yamamoto, and S. Tabata. 2004. Val216 decides the substrate specificity of alpha-glucosidase in Saccharomyces cerevisiae. European Journal of Biochemistry 271:3414-3420.
100. Zhang, J. B., P. Kowal, X. Chen, and P. G. Wang. 2003. Large-scale synthesis of globotriose derivatives through recombinant E. coli. Organic & Biomolecular Chemistry 1:3048-3053.
101. Zhao, J., T. Baba, H. Mori, and K. Shimizu. 2004. Global metabolic response of Escherichia coli to gnd or zwf gene-knockout, based on 13C-labeling experiments and the measurement of enzyme activities. Applied Microbiology and Biotechnology 64:91-98.

1-29. (canceled)

30. A metabolically engineered bacterium or yeast for the production of a specialty product selected from the group consisting of a carbohydrate specialty product, an O-glycoside or a glycolipid, characterized in that said bacterium or yeast:
a) has been genetically modified by introducing a heterologous gene encoding a sucrose synthase capable of splitting sucrose into UDP-glucose and fructose; and
b) has been further genetically modified to prevent loss of UDP-glucose via glycolysis due to the genetic disruption of an endogenous gene encoding an UDP-glucose hydrolase, an UDP-glucose-4-epimerase, a glucose-1-phosphate uridilyltransferase, an UDP-glucose 6-dehydrogenase, an UDP-glucose diphosphorylase, an UDP-glucose-hexose-1-phosphate uridyltransferase, an UDP-glucose-glycogen glucosyltransferase, an UDP-glucose-1,3-beta-D-glucan glucosyltransferase, an UDP-glucose-glucosephosphate glucosyltransferase, or a combination thereof.

31. The metabolically engineered bacterium or yeast according to claim 30, wherein an endogenous gene encoding an UDP-glucose hydrolase, an endogenous gene encoding an UDP-glucose-hexose-1-phosphate uridyltransferase, an endogenous gene encoding an UDP-glucose-4-epimerase, an endogenous gene encoding a glucose-1-phosphate uridilyltransferase and an endogenous gene encoding an UDP-glucose 6-dehydrogenase have been disrupted.

32. The metabolically engineered bacterium or yeast according to claim 30, wherein an endogenous gene encoding an UDP-glucose diphosphorylase, an endogenous gene encoding an UDP-glucose 4-epimerase, an endogenous gene encoding an UDP-glucose-hexose-1-phosphate uridyltransferase, an endogenous gene encoding an UDP-glucose-glycogen glucosyltransferase, an endogenous gene encoding an UDP-glucose-1,3-beta-D-glucan glucosyltransferase and an endogenous gene encoding an UDP-glucose-glucosephosphate glucosyltransferasehave been disrupted.

33. The metabolically engineered bacterium or yeast according to claim 30, wherein said UDP-glucose-hexose-1-phosphate uridyltransferase is an UDP-glucose-galactose-1-phosphate uridyltransferase.

34. The metabolically engineered bacterium or yeast according to claim 30, wherein said sucrose synthase is from Solamim tuberosum.

35. The metabolically engineered bacterium or yeast according to claim 30, wherein said carbohydrate specialty product is an activated saccharide or a disaccharide.

36. The metabolically engineered bacterium or yeast according to claim 35, wherein said activated saccharide is UDP-glucose.

37. A method for the production of a carbohydrate specialty product, comprising the steps of:
i) cultivating the metabolically engineered bacterium or yeast according to claim 30; and
ii) extracting and purifying the carbohydrate specialty product.

38. A method for the production of a disaccharide, comprising the steps of:
i) cultivating the metabolically engineered bacterium or yeast according to claim 30; and
ii) extracting and purifying the disaccharide.

39. A method for the production of UDP-glucose, comprising the steps of:
i) cultivating the metabolically engineered bacterium or yeast according to claim 30; and
ii) extracting and purifying the UDP-glucose.

40. The metabolically engineered bacterium or yeast according to claim 35, wherein said disaccharide is sophorose.

41. The metabolically engineered bacterium or yeast according to claim 30, wherein said O-glycoside is flavone 3-O-β-D-glucoside.

42. The metabolically engineered bacterium or yeast according to claim 30, wherein said bacterium or yeast comprises one or more enzymes which convert said UDP-glucose into said specialty product.

43. The metabolically engineered bacterium or yeast according to claim 41, wherein said bacterium or yeast has been further genetically modified by introducing one or more heterologous genes encoding one or more enzymes which convert said UDP-glucose into said specialty product.

44. The metabolically engineered bacterium or yeast according to claim 30, wherein said specialty product is sophorose and wherein said bacterium or yeast comprises a β-glucosyltransferase to catalyze transfer of glucose from UDP-glucose to a glucose in a beta linkage.

45. The metabolically engineered bacterium or yeast according to claim 43, wherein said bacterium or yeast has been further genetically modified by introducing a heterologous gene encoding a β-glucosyltransferase.

46. The metabolically engineered bacterium or yeast according to claim 44, wherein said β-glucosyltransferase is from Streptococcus pneumoniae.

47. A method for the production of sophorose, comprising the steps of:
i) cultivating the metabolically engineered bacterium or yeast according to claim 43; and
ii) extracting and purifying the sophorose.

48. The metabolically engineered bacterium or yeast according to claim 30, wherein said bacterium is Escherichia coli or wherein said yeast is Saccharomyces cerevisiae.

49. The metabolically engineered bacterium or yeast according to claim 30, wherein the metabolically engineered bacterium or yeast is a metabolically engineered bacterium, optionally wherein the bacterium is Escherichia coli.

50. The metabolically engineered bacterium or yeast according to claim 30, wherein the metabolically engineered bacterium or yeast is a metabolically engineered yeast, optionally wherein the yeast is Saccharomyces cerevisiae.


SEQUENCE LISTING (56 sequences)

SEQ ID NO: 1 - protein, 768 aa, Dictyostellium discoideum
MNDSPIISVVLPFLIKDNDDKSLNYQGINNLIISIDSIIEQTFKEWELILVDDGSNNEILEQLLSKRYSTDNRIKFIINKENKGIVKSLNDAILNHCSPTSKYIARMDSDDISHPTRLQSQLKYLQSNETIDILGCPIKMFNNNKLIEILNNNNNNNNINNNVKELINIINNEESFKFIQHPDKDILMWSMFFNCCIVHPSVIFKRSIFTIEHCYEENNQFPFIEDYLFWLKSLIMKGLNISNIQSSTPLLYLRKHNNSISFKNIEKQKDSTANASCYYLNILFKRFNIDSEIIQNSSLSMKEIIQFFQLSPSSLSKINNISIELFEFAFKYLELIEKSCTKQQPNYSNSIKDAANEKMGELVSLCLSNYPNNQKSSLLWEKWLSRNPTSQLLSLLSNLNVKSSTTIINNNINNNNNNNNNNNNNNNNNNNNNNNNNNNNSILNFISGINSNKINTPKSNNNKFKENGIRIICFSKDRAFQLKEYLRTFFKYLKNDDNGNDKFEIIVDVLFTYSNEKFKNSYQLVIESFPQVNFIKEENFTDQLINLVQKTNKLEYVMFSVDDILYYNEFNLKEYCLSLNSEPLALGFYMKLNKNITYCHTCNQDITIPLNSNTISRTENNFKYLKWNRNDNDCKKDWNYPWDLCSTIYRCNDIDSIINGIVKYYGIRNGINHPNRFEFNGNRPIIQKQIYQNKPYCLCLSDHYSPMSVVTINRVQDVYDNPIYDQTLSLDDLDQLLYSNKSLNDEKYKENSLSLNFKSVHIGELFIS

SEQ ID NO: 2 - DNA, 2307 bp, synthetic construct
  misc_feature 1..2307: note = codon optimized sequence encoding SEQ ID NO 1
  source 1..2307: mol_type = other DNA
atgaacgatagcccgattattagcgttgttctgccgtttctgatcaaagataacgatgataaaagcctgaactaccagggcattaacaacctgattattagcatcgatagcatcatcgagcagacctttaaagaatgggaactgattctggttgatgatggcagcaataacgaaattctggaacagctgctgagcaaacgttatagcaccgataaccgcatcaaatttattattaataaagaaaataaaggcattgtgaaaagcctgaatgatgccattctgaatcattgtagcccgaccagcaaatatattgcacgtatggatagcgacgatattagccatccgacccgtctgcagagccagctgaaatatctgcagagcaatgaaaccattgatattctgggttgcccgatcaaaatgtttaataataataaactgattgaaattctgaataataataacaataacaacaatattaataataatgtgaaagaactgattaatattattaataatgaagaaagctttaaatttattcagcatccggataaagatattctgatgtggtccatgttcttcaattgctgtattgttcatccgagcgtgatttttaaacgcagcatttttaccatcgagcactgctatgaagagaataatcagtttccgttcatcgaggattacctgttttggctgaaatccctgattatgaaaggcctgaacattagcaatatccagagcagcacaccgctgctgtatctgcgtaaacataataacagcattagctttaaaaatattgaaaaacagaaagatagcaccgccaatgccagctgttattatctgaacattctgttcaaacgctttaacatcgacagcgaaattattcagaatagcagcctgagcatgaaagaaatcatccagttttttcagctgagcccgagcagcctgtccaaaattaataacattagcatcgaactgtttgaatttgcctttaaatatctggaactgatcgagaaaagctgtaccaaacagcagccgaattatagcaacagcattaaagatgcagccaacgaaaaaatgggtgaactggttagcctgtgtctgagcaattatccgaataatcagaaaagcagtctgctgtgggaaaaatggctgagccgtaatccgaccagccagctgctgagtctgctgagcaatctgaatgttaaaagcagcaccaccattattaataacaatattaacaacaacaataataataacaacaataataacaataacaataacaataataacaacaacaacaataataataataacaacaacagcattctgaattttattagcggcattaatagcaataaaattaataccccgaaaagcaacaataacaaatttaaagagaatggcattcgcattatttgcttcagcaaagatcgtgcattccagctgaaagaatatctgcgcaccttcttcaaatatctgaaaaatgatgataatggcaatgataaatttgaaattattgtggatgtgctgtttacctatagcaatgaaaaattcaaaaatagctatcagctggtgatcgaaagctttccgcaggttaactttattaaagaagaaaactttaccgatcagctgattaacctggtgcagaaaaccaacaaactggaatatgtgatgttcagcgtggatgatatcctgtattacaacgagttcaatctgaaagagtattgcctgagcctgaatagcgaaccgctggcactgggtttttatatgaaactgaataaaaatattacctattgccatacctgcaaccaggatattaccattccgctgaatagcaataccattagccgcaccgaaaataactttaaatacctgaaatggaatcgcaacgataatgattgcaaaaaagactggaactatccgtgggatctgtgtagcaccatttatcgttgcaacgacattgacagcatcattaatggtattgtgaaatattatggtattcgcaacggcattaatcatccgaatcgctttgaatttaatggcaaccgtccgattattcagaaacaaatctaccagaacaaaccgtattgtctgtgcctgagcgatcattattcaccgatgagcgttgttaccattaatcgtgttcaggatgtgtatgataacccgatttatgatcagaccctgagcctggatgatctggatcaactgctgtatagcaataaatccctgaacgatgaaaaatataaagaaaacagcctgagtctgaacttcaaaagcgttcatattggcgaactgttcatcagctaa

SEQ ID NO: 3 - DNA, 2307 bp, Dictyostellium discoideum
  source 1..2307: mol_type = genomic DNA
atgaatgattcaccaataataagtgtagttttaccttttttaataaaggacaatgacgataaatcattaaattaccaaggaataaataatttaataatatcaatagatagcattattgaacaaacttttaaagaatgggaattaattttagttgatgatggatcaaataatgaaattttggagcaattactttcaaaaagatatagtacagataatagaattaaattcataataaataaagagaataaaggtattgttaaaagtttaaatgatgcaattttaaatcattgttcaccaacttcaaaatatattgctcgtatggattcagatgatatttctcatccaacaagattacaatctcaacttaaatatcttcaatcaaatgaaacaattgatatattaggttgtccaattaaaatgtttaataataataaattaattgaaattttaaataataataataataataataatattaataataatgtgaaagagttaattaatataattaataatgaagaatcttttaaatttattcaacatcctgataaagatattttaatgtggtcaatgtttttcaattgttgtattgttcacccttctgtaatatttaaaagatcgatattcactattgaacattgttatgaagaaaacaaccaatttccattcattgaagattacttattttggttaaaatccttaataatgaaaggtttaaatatttcaaatatccaatcatcaacaccattactatatttaagaaaacataataactctatatcttttaaaaatattgaaaaacaaaaagattccactgctaatgcatcttgttattatctaaatatactttttaaaagatttaatattgattctgaaattattcaaaattcttcactctcaatgaaagaaattattcagttctttcaactttcaccatcatctttatcaaaaatcaataatatttcaattgaattatttgaatttgcatttaaatatctagaattaattgaaaaatcatgtacaaaacaacaaccaaactattcaaacagtataaaagatgcagcaaatgaaaaaatgggtgaattagtatctttatgtttatcaaattatccaaataatcaaaaatcatcattactttgggaaaaatggttatcaagaaatccaacctcacaattactatcacttttatcaaatttaaatgtaaaatcttcaactactataattaataataatattaataataataataataataataataataataataataataataataataataataataataataataataataataataataattcaattttaaattttatatctggcattaatagtaataaaataaatactccaaaatctaataataataaatttaaagaaaatggaattagaataatttgtttctcaaaagatagagcatttcaattaaaagaatatcttagaacattttttaaatatttaaaaaatgatgataatggaaatgataaatttgaaattattgttgatgtattatttacatattcaaatgagaaattcaaaaactcttatcaattagttattgaaagttttccacaagttaattttattaaagaagagaatttcactgatcaattaattaatttagttcaaaaaacaaataaacttgaatatgtcatgttttcagttgatgatattctttattataatgaattcaatctcaaagaatattgtttatctttgaatagtgagccattggcattaggtttctatatgaagttaaataaaaatattacctattgtcatacttgtaatcaagatataacaataccattaaattcaaatactattagtagaacagagaataattttaaatatttaaaatggaatagaaatgataatgattgtaaaaaggattggaattatccatgggatttatgttcaaccatttatagatgtaatgatattgattcaatcattaatggtatagttaaatattatggaattagaaatggtattaatcatccaaatagattcgaattcaatggtaatagaccaatcattcaaaagcaaatctatcaaaataaaccctactgtttatgtttatcagatcactattctccaatgtctgttgtaactattaatagagttcaagatgtctatgataatccaatttatgaccaaaccctatctttagatgatttagatcaattactttattcaaacaaatcattaaatgatgaaaaatataaagaaaatagtttatctttaaattttaaaagtgttcatattggtgaactttttatttcttaa

SEQ ID NO: 4 - protein, 329 aa, Dictyostellium discoideum
MSILNFISGINSNKINTPKSNNNKFKENGIRIICFSKDRAFQLKEYLRTFFKYLKNDDNGNDKFEIIVDVLFTYSNEKFKNSYQLVIESFPQVNFIKEENFTDQLINLVQKTNKLEYVMFSVDDILYYNEFNLKEYCLSLNSEPLALGFYMKLNKNITYCHTCNQDITIPLNSNTISRTENNFKYLKWNRNDNDCKKDWNYPWDLCSTIYRCNDIDSIINGIVKYYGIRNGINHPNRFEFNGNRPIIQKQIYQNKPYCLCLSDHYSPMSVVTINRVQDVYDNPIYDQTLSLDDLDQLLYSNKSLNDEKYKENSLSLNFKSVHIGELFIS

SEQ ID NO: 5 - DNA, 990 bp, synthetic construct
  misc_feature 1..990: note = codon optimized sequence encoding part of SEQ ID NO 2
  source 1..990: mol_type = other DNA
atgagcattctgaattttattagcggcattaatagcaataaaattaataccccgaaaagcaacaataacaaatttaaagagaatggcattcgcattatttgcttcagcaaagatcgtgcattccagctgaaagaatatctgcgcaccttcttcaaatatctgaaaaatgatgataatggcaatgataaatttgaaattattgtggatgtgctgtttacctatagcaatgaaaaattcaaaaatagctatcagctggtgatcgaaagctttccgcaggttaactttattaaagaagaaaactttaccgatcagctgattaacctggtgcagaaaaccaacaaactggaatatgtgatgttcagcgtggatgatatcctgtattacaacgagttcaatctgaaagagtattgcctgagcctgaatagcgaaccgctggcactgggtttttatatgaaactgaataaaaatattacctattgccatacctgcaaccaggatattaccattccgctgaatagcaataccattagccgcaccgaaaataactttaaatacctgaaatggaatcgcaacgataatgattgcaaaaaagactggaactatccgtgggatctgtgtagcaccatttatcgttgcaacgacattgacagcatcattaatggtattgtgaaatattatggtattcgcaacggcattaatcatccgaatcgctttgaatttaatggcaaccgtccgattattcagaaacaaatctaccagaacaaaccgtattgtctgtgcctgagcgatcattattcaccgatgagcgttgttaccattaatcgtgttcaggatgtgtatgataacccgatttatgatcagaccctgagcctggatgatctggatcaactgctgtatagcaataaatccctgaacgatgaaaaatataaagaaaacagcctgagtctgaacttcaaaagcgttcatattggcgaactgttcatcagctaa

SEQ ID NO: 6 - DNA, 987 bp, Dictyostellium discoideum
  source 1..987: mol_type = genomic DNA
tcaattttaaattttatatctggcattaatagtaataaaataaatactccaaaatctaataataataaatttaaagaaaatggaattagaataatttgtttctcaaaagatagagcatttcaattaaaagaatatcttagaacattttttaaatatttaaaaaatgatgataatggaaatgataaatttgaaattattgttgatgtattatttacatattcaaatgagaaattcaaaaactcttatcaattagttattgaaagttttccacaagttaattttattaaagaagagaatttcactgatcaattaattaatttagttcaaaaaacaaataaacttgaatatgtcatgttttcagttgatgatattctttattataatgaattcaatctcaaagaatattgtttatctttgaatagtgagccattggcattaggtttctatatgaagttaaataaaaatattacctattgtcatacttgtaatcaagatataacaataccattaaattcaaatactattagtagaacagagaataattttaaatatttaaaatggaatagaaatgataatgattgtaaaaaggattggaattatccatgggatttatgttcaaccatttatagatgtaatgatattgattcaatcattaatggtatagttaaatattatggaattagaaatggtattaatcatccaaatagattcgaattcaatggtaatagaccaatcattcaaaagcaaatctatcaaaataaaccctactgtttatgtttatcagatcactattctccaatgtctgttgtaactattaatagagttcaagatgtctatgataatccaatttatgaccaaaccctatctttagatgatttagatcaattactttattcaaacaaatcattaaatgatgaaaaatataaagaaaatagtttatctttaaattttaaaagtgttcatattggtgaactttttatttcttaa

SEQ ID NO: 7 - DNA, 74 bp, synthetic construct
  misc_feature 1..74: note = Primer
  source 1..74: mol_type = other DNA
cataatggatttccttacgcgaaatacgggcagacatggcctgcccggttattagtgtaggctggagctgcttc

SEQ ID NO: 8 - DNA, 71 bp, synthetic construct
  misc_feature 1..71: note = Primer
  source 1..71: mol_type = other DNA
gtatgttgtgtggaattgtgagcggataacaatttcacacaggaaacagctcatatgaatatcctccttag

SEQ ID NO: 9 - DNA, 75 bp, synthetic construct
  misc_feature 1..75: note = Primer
  source 1..75: mol_type = other DNA
agaccgccggttttaagcagcgggaacatctctgaacatacatgtaaaacctgcagtgtaggctggagctgcttc

SEQ ID NO: 10 - DNA, 82 bp, synthetic construct
  misc_feature 1..82: note = Primer
  source 1..82: mol_type = other DNA
gtctggcagggacctgcacacggattgtgtgtgttccagagatgataaaaaaggagttagtccatatgaatatcctccttag

SEQ ID NO: 11 - DNA, 70 bp, synthetic construct
  misc_feature 1..70: note = Primer
  source 1..70: mol_type = other DNA
catatttctgtcacactctttagtgattgataacaaaagaggtgccaggagtgtaggctggagctgcttc

SEQ ID NO: 12 - DNA, 70 bp, synthetic construct
  misc_feature 1..70: note = Primer
  source 1..70: mol_type = other DNA
taaaaacgtttaaccagcgactcccccgcttctcgcgggggagttttctgcatatgaatatcctccttag

SEQ ID NO: 13 - DNA, 70 bp, synthetic construct
  misc_feature 1..70: note = Primer
  source 1..70: mol_type = other DNA
ggcgctacaatcttccaaagtcacaattctcaaaatcagaagagtattgcgtgtaggctggagctgcttc

SEQ ID NO: 14 - DNA, 70 bp, synthetic construct
  misc_feature 1..70: note = Primer
  source 1..70: mol_type = other DNA
ggttgccggatgcggcgtgaacgccttatccggcctacatatcgacgatgcatatgaatatcctccttag

SEQ ID NO: 15 - DNA, 69 bp, synthetic construct
  misc_feature 1..69: note = Primer
  source 1..69: mol_type = other DNA
gacttccggcaacagatttcattttgcattccaaagttcagaggtagtcgtgtaggctggagctgcttc

SEQ ID NO: 16 - DNA, 69 bp, synthetic construct
  misc_feature 1..69: note = Primer
  source 1..69: mol_type = other DNA
gcttctgtcatcggtttcagggtaaaggaatctgcctttttccgaaatccatatgaatatcctccttag

SEQ ID NO: 17 - DNA, 71 bp, synthetic construct
  misc_feature 1..71: note = Primer
  source 1..71: mol_type = other DNA
cactttccgctgattcggtgccagactgaaatcagcctataggaggaaatggtgtaggctggagctgcttc

SEQ ID NO: 18 - DNA, 70 bp, synthetic construct
  misc_feature 1..70: note = Primer
  source 1..70: mol_type = other DNA
gttgccgacaggttggtgatgattcccccaatgctgggggaatgtttttgcatatgaatatcctccttag

SEQ ID NO: 19 - DNA, 70 bp, synthetic construct
  misc_feature 1..70: note = Primer
  source 1..70: mol_type = other DNA
tgagaaggtttgcggaactatctaaaacgttgcagacaaaggacaaagcagtgtaggctggagctgcttc

SEQ ID NO: 20 - DNA, 71 bp, synthetic construct
  misc_feature 1..71: note = Primer
  source 1..71: mol_type = other DNA
catacgtaaaaaagggcgatcttgcgaccgccctttttttattaaatgtgtcatatgaatatcctccttag

SEQ ID NO: 21 - DNA, 70 bp, synthetic construct
  misc_feature 1..70: note = Primer
  source 1..70: mol_type = other DNA
tgagaaggtttgcggaactatctaaaacgttgcagacaaaggacaaagcaacgaaaggctcagtcgaaag

SEQ ID NO: 22 - DNA, 71 bp, synthetic construct
  misc_feature 1..71: note = Primer
  source 1..71: mol_type = other DNA
catacgtaaaaaagggcgatcttgcgaccgccctttttttattaaatgtgtagaactccagcatgagatcc

SEQ ID NO: 23 - DNA, 70 bp, synthetic construct; identical to SEQ ID NO: 19
  misc_feature 1..70: note = Primer
  source 1..70: mol_type = other DNA

SEQ ID NO: 24 - DNA, 71 bp, synthetic construct
  misc_feature 1..71: note = Primer
  source 1..71: mol_type = other DNA
catacgtaaaaaagggcgatcttgcgaccgccctttttttattaaatgtgtcatccgtcaggatggccttc

SEQ ID NO: 25 - DNA, 72 bp, synthetic construct
  misc_feature 1..72: note = Primer
  source 1..72: mol_type = other DNA
gccacgcgtgagaacgtaaaaaaagcacccatactcaggagcactctcaattgtgtaggctggagctgcttc

SEQ ID NO: 26 - DNA, 75 bp, synthetic construct
  misc_feature 1..75: note = Primer
  source 1..75: mol_type = other DNA
cacctgtaaaaaaggcagccatctggctgccttagtctccccaacgtcttacggacatatgaatatcctccttag

SEQ ID NO: 27 - DNA, 70 bp, synthetic construct
  misc_feature 1..70: note = Primer
  source 1..70: mol_type = other DNA
cgagaaggcccggattgtcatggacgatgagatacaccggaatatcatgggtgtaggctggagctgcttc

SEQ ID NO: 28 - DNA, 76 bp, synthetic construct
  misc_feature 1..76: note = Primer
  source 1..76: mol_type = other DNA
ccaggtatttacagtgtgagaaagaattattttgactttagcggagcagttgaagacatatgaatatcctccttag

SEQ ID NO: 29 - DNA, 70 bp, synthetic construct
  misc_feature 1..70: note = Primer
  source 1..70: mol_type = other DNA
atatccagccagtcttccggctgtagtcctaacagagcactgttactgtcagcattacacgtcttgagcg

SEQ ID NO: 30 - DNA, 70 bp, synthetic construct
  misc_feature 1..70: note = Primer
  source 1..70: mol_type = other DNA
gctttaagtggttgagatcacatttccttgctcatccccgcaactcctcccatatgaatatcctccttag

SEQ ID NO: 31 - DNA, 68 bp, synthetic construct
  misc_feature 1..68: note = Primer
  source 1..68: mol_type = other DNA
atatccagccagtcttccggctgtagtcctaacagagcactgttactgtcgtaaaacgacggccagtg

SEQ ID NO: 32 - DNA, 72 bp, synthetic construct
  misc_feature 1..72: note = Primer
  source 1..72: mol_type = other DNA
caacggccattttttgcacttagatacagattttctgcgctgtattgcattgccgggatccgatgcatatgg

SEQ ID NO: 33 - DNA, 75 bp, synthetic construct
  misc_feature 1..75: note = Primer
  source 1..75: mol_type = other DNA
ttttattttgcccttcaatgggaccgctaccaaacatcaggaggatgaatgaaacagcattacacgtcttgagcg

SEQ ID NO: 34 - DNA, 70 bp, synthetic construct
  misc_feature 1..70: note = Primer
  source 1..70: mol_type = other DNA
ttccgttgaaggcaacagtaattgcgccccggttaagcccgcgccgatcccatatgaatatcctccttag

SEQ ID NO: 35 - DNA, 71 bp, synthetic construct
  misc_feature 1..71: note = Primer
  source 1..71: mol_type = other DNA
gtagcattgttcctaagtatgactccatttttccaggaatggtcgcaaatcgtgtaggctggagctgcttc

SEQ ID NO: 36 - DNA, 70 bp, synthetic construct
  misc_feature 1..70: note = Primer
  source 1..70: mol_type = other DNA
ttcacgccgcatccggcaagcaaaccagctcataagccgggagaacaacccatatgaatatcctccttag

SEQ ID NO: 37 - DNA, 70 bp, synthetic construct
  misc_feature 1..70: note = Primer
  source 1..70: mol_type = other DNA
ttcacgccgcatccggcaagcaaaccagctcataagccgggagaacaaccccgcttacagacaagctgtg

SEQ ID NO: 38 - DNA, 71 bp, synthetic construct
  misc_feature 1..71: note = Primer
  source 1..71: mol_type = other DNA
gtagcattgttcctaagtatgactccatttttccaggaatggtcgcaaatcagccatgacccgggaattac

SEQ ID NO: 39 - DNA, 70 bp, synthetic construct
  misc_feature 1..70: note = Primer
  source 1..70: mol_type = other DNA
atcgccgaccacttcgcgccgctgatggttttttcacgtaagctcatatcccgcttacagacaagctgtg

SEQ ID NO: 40 - DNA, 70 bp, synthetic construct
  misc_feature 1..70: note = Primer
  source 1..70: mol_type = other DNA
ggatcttcccttaccccactgcgggtaaggggctaataacaggaacaacgagccatgacccgggaattac

SEQ ID NO: 41 - DNA, 42 bp, synthetic construct
  misc_feature 1..42: note = Primer
  source 1..42: mol_type = other DNA
gggggcccccgggggtatgagcttacgtgaaaaaaccatcag

SEQ ID NO: 42 - DNA, 43 bp, synthetic construct
  misc_feature 1..43: note = Primer
  source 1..43: mol_type = other DNA
gggcccgggcccgggcgttgttcctgttattagccccttaccc

SEQ ID NO: 43 - DNA, 70 bp, synthetic construct
  misc_feature 1..70: note = Primer
  source 1..70: mol_type = other DNA
atcgccgaccacttcgcgccgctgatggttttttcacgtaagctcatatcgtgtaggctggagctgcttc

SEQ ID NO: 44 - DNA, 70 bp, synthetic construct
  misc_feature 1..70: note = Primer
  source 1..70: mol_type = other DNA
ggatcttcccttaccccactgcgggtaaggggctaataacaggaacaacgcatatgaatatcctccttag

SEQ ID NO: 45 - DNA, 70 bp, synthetic construct
  misc_feature 1..70: note = Primer
  source 1..70: mol_type = other DNA
ttttgatatcgaaccagacgctccattcgcggatgtactcaaggtcgaacgtgtaggctggagctgcttc

SEQ ID NO: 46 - DNA, 70 bp, synthetic construct
  misc_feature 1..70: note = Primer
  source 1..70: mol_type = other DNA
tctatggtgcaacgcttttcagatatcaccatcatgtttgccggactatgcatatgaatatcctccttag

SEQ ID NO: 47 - DNA, 70 bp, synthetic construct
  misc_feature 1..70: note = Primer
  source 1..70: mol_type = other DNA
tagccaaatgcgttggcaaacagagattgtgttttttctttcagactcatctttgtttcctccgaattcg

SEQ ID NO: 48 - DNA, 70 bp, synthetic construct
  misc_feature 1..70: note = Primer
  source 1..70: mol_type = other DNA
cggttcgacgcatgcaggcatgaaaccgcgtcttttttcagataaaaagccatatgaatatcctccttag

SEQ ID NO: 49 - DNA, 70 bp, synthetic construct
  misc_feature 1..70: note = Primer
  source 1..70: mol_type = other DNA
accaatcaaattcacgcggccaggcgcctgaatggtgtgagtggcagggtagccaaatgcgttggcaaac

SEQ ID NO: 50 - DNA, 70 bp, synthetic construct
  misc_feature 1..70: note = Primer
  source 1..70: mol_type = other DNA
gtcggtagtgctgaccttgccggaggcggccttagcaccctctccggccaacggttcgacgcatgcaggc

SEQ ID NO: 51 - DNA, 1841 bp, Escherichia coli
  source 1..1841: mol_type = genomic DNA
agcatgatcgaacacatcatgcagtcgatgcaattcccggcggaattgattgagaaggtttgcggaactatctaaaacgttgcagacaaaggacaaagcaatggcaatccacaatcgtgcaggccaacctgcacaacagagtgatttgattaacgtcgcccaactgacggcgcaatattatgtactgaaaccagaagcagggaatgcggagcacgcggtgaaattcggtacttccggtcaccgtggcagtgcagcgcgccacagctttaacgagccgcacattctggcgatcgctcaggcaattgctgaagaacgtgcgaaaaacggcatcactggcccttgctatgtgggtaaagatactcacgccctgtccgaacctgcattcatttccgttctggaagtgctggcagcgaacggcgttgatgtcattgtgcaggaaaacaatggcttcaccccgacgcctgccgtttccaatgccatcctggttcacaataaaaaaggtggcccgctggcagacggtatcgtgattacaccgtcccataacccgccggaagatggtggaatcaaatacaatccgccaaatggtggcccggctgataccaacgtcactaaagtggtggaagacagggccaacgcactgctggccgatggcctgaaaggcgtgaagcgtatctccctcgacgaagcgatggcatccggtcatgtgaaagagcaggatctggtgcagccgttcgtggaaggtctggccgatatcgttgatatggccgcgattcagaaagcgggcctgacgctgggcgttgatccgctgggcggttccggtatcgaatactggaagcgtattggcgagtattacaacctcaacctgactatcgttaacgatcaggtcgatcaaaccttccgctttatgcaccttgataaagacggcgcgatccgtatggactgctcctccgagtgtgcgatggcgggcctgctggcactgcgtgataagttcgatctggcgtttgctaacgacccggattatgaccgtcacggtatcgtcactccggcaggtttgatgaatccgaaccactacctggcggtggcaatcaattacctgttccagcatcgtccgcagtggggcaaagatgttgccgtcggtaaaacgctggtttcatctgcgatgatcgaccgtgtggtcaacgacttgggccgtaaactggtagaagtcccggtaggtttcaaatggtttgtcgatggtctgttcgacggcagcttcggctttggcggcgaagagagtgcaggggcttccttcctgcgtttcgacggcacgccgtggtccaccgacaaagacggcatcatcatgtgtctgctggcggcggaaatcaccgctgtcaccggtaagaacccgcaggaacactacaacgaactggcaaaacgctttggtgcgccgagctacaaccgtttgcaggcagctgcgacttccgcacaaaaagcggcgctgtctaagctgtctccggaaatggtgagcgccagcaccctggcaggtgacccgatcaccgcgcgcctgactgctgctccgggcaacggtgcttctattggcggtctgaaagtgatgactgacaacggctggttcgccgcgcgtccgtcaggcacggaagacgcatataagatctactgcgaaagcttcctcggtgaagaacatcgcaagcagattgagaaagaagcggttgagattgttagcgaagttctgaaaaacgcgtaaacacatttaataaaaaaagggcggtcgcaagatcgcccttttttacgtatgacaaacacagaattgcctgatgcgctacgcttatcaggcctacgaggat

SEQ ID NO: 52 - DNA, 509 bp, Escherichia coli
  source 1..509: mol_type = genomic DNA
cgtacacgcgtttactttgcggcagatgaacaaacgctgctgaaaaatggtaatcagaccaagccgaaacatgtgccaggcacgccgtattgggtgatcaccaacaccaacaccggccgtaaatgcagcatgatcgaacacatcatgcagtcgatgcaattcccggcggaattgattgagaaggtttgcggaactatctaaaacgttgcagacaaaggacaaagcagtgtaggctggagctgcttcgaagttcctatactttctagagaataggaacttcggaataggaactaaggaggatattcatatgacacatttaataaaaaaagggcggtcgcaagatcgcccttttttacgtatgacaaacacagaattgcctgatgcgctacgcttatcaggcctacgaggatggtgcaatatattgaatttaagcgattttgtaggccggataaggcgttcacgccgcatccggcaaaaacaacgaacactttgtcaacaaactgagtagc

SEQ ID NO: 53 - DNA, 2061 bp, Escherichia coli
  misc_feature 707: note = n is a, c, g, or t
  source 1..2061: mol_type = genomic DNA
cgtacacgcgtttactttgcggcagatgaacaaacgctgctgaaaaatggtaatcagaccaagccgaaacatgtgccaggcacgccgtattgggtgatcaccaacaccaacaccggccgtaaatgcagcatgatcgaacacatcatgcagtcgatgcaattcccggcggaattgattgagaaggtttgcggaactatctaaaacgttgcagacaaaggacaaagcagtgtaggctggagctgcttcgaagttcctatactttctagagaataggaacttcggaataggaactaaggaggatattcatatggaccgatatcccgggcggccgcttcatttataaatttcttgacattttggaatagatgtgatataatgtgtacatatccatggcggccgctctagaagaagcttgggatccgtcgacctcgaattcggaggaaacaaagatggggggttctcatcatcatcatcatcatggtatggctagcatgagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttcgggtatggtgttcaatgctttgcnagatacccagatcatatgaaacagcatgactttttcaagagtgccatgcccgaaggttatgtacaggaaagaactatatttttcaaagatgacgggaactacaagacacgtgctgaagtcaagtttgaaggtgatacccttgttaatagaatcgagttaaaaggtattgattttaaagaagatggaaacattcttggacacaaattggaatacaactataactcacacaatgtatacatcatggcagacaaacaaaagaatggaatcaaagttaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaataactgcaggtcgaccatatgggagagctcccaacgcgttggatgcaggcatgcaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatgctactgcgaaagcttcctcggtgaagaacatcgcaagcagattgagaaagaagcggttgagattgttagcgaagttctgaaaaacgcgtaactactgcgaaagcttcctcggtgaagaacatcgcaagcagattgagaaagaagcggttgagattgttagcgaagttctgaaaaacgcgtaaacacatttaataaaaaaagggcggtcgcaagatcgcccttttttacgtatgacaaacacagaattgcctgatgcgctacgcttatcaggcctacgaggatggtgcaatatattgaatttaagcgattttgtaggccggataaggcgttcacgccgcatccggcaaaaacaacgaacactttgtcaacaaactgagtagctcaaggaaatccca

SEQ ID NO: 54 - DNA, 1735 bp, Escherichia coli
  source 1..1735: mol_type = genomic DNA
cgtacacgcgtttactttgcggcagatgaacaaacgctgctgaaaaatggtaatcagaccaagccgaaacatgtgccaggcacgccgtattgggtgatcaccaacaccaacaccggccgtaaatgcagcatgatcgaacacatcatgcagtcgatgcaattcccggcggaattgattgagaaggtttgcggaactatctaaaacgttgcagacaaaggacaaagcaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtggccagtgccaagcttgcatgcagattgcagcattacacgtcttgagcgattgtgtaggctggagctgcttcgaagttcctatactttctagagaataggaacttcggaataggaacttcaagatcccctcacgctgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcaagatcccctcacgctgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcgcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttctctactgcgaaagcttcctcggtgaagaacatcgcaagcagattgagaaagaagcggttgagattgttagcgaagttctgaaaaacgcgtaaacacatttaataaaaaaagggcggtcgcaagatcgcccttttttacgtatgacaaacacagaattgcctgatgcgctacgcttatcaggcctacgaggatggtgcaatatattgaatttaagcgattttgtaggccggataaggcgttcacgccgcatccggcaaaaacaacgaacactttgtcaacaaactgagtagctcaaggaaatccca

SEQ ID NO: 55 - protein, 328 aa, Dictyostellium discoideum
SILNFISGINSNKINTPKSNNNKFKENGIRIICFSKDRAFQLKEYLRTFFKYLKNDDNGNDKFEIIVDVLFTYSNEKFKNSYQLVIESFPQVNFIKEENFTDQLINLVQKTNKLEYVMFSVDDILYYNEFNLKEYCLSLNSEPLALGFYMKLNKNITYCHTCNQDITIPLNSNTISRTENNFKYLKWNRNDNDCKKDWNYPWDLCSTIYRCNDIDSIINGIVKYYGIRNGINHPNRFEFNGNRPIIQKQIYQNKPYCLCLSDHYSPMSVVTINRVQDVYDNPIYDQTLSLDDLDQLLYSNKSLNDEKYKENSLSLNFKSVHIGELFIS

SEQ ID NO: 56 - protein, 300 aa, Helicobacter pylori
MAFKVVRICGGLGNQMFQYAFAKSLQKHSNTPVLLDITSFDWSNRKMQLELFPIDLPYASAKEIAIAKMQHLPKLVRDALKYIGFDRVSQEIVFEYEPKLLKPSRLTYFYGYFQDPRYFDAISPLIKQTFTLPPPPKIIRIIIKKEEEYHRKLALILAAKNSVFVHIRRGDYVGIGCQLGIDYQKKALEYMAKRVPNMELFVFCEDLTFTQNLDLGYPFMDMTTRDKDEEAYWDMLLMQSCQHGIIANSTYSWWAAYLIENPEKIIIGPKHWLFGHENILCKEWVKIESHFEVKSQKYNA